SEQUENCE LISTING (88 sequences)

SEQ ID NO: 1 - protein, 8 aa, Artificial sequence
  Synthetic
SIINFEKL

SEQ ID NO: 2 - DNA, 23 bp, Mus musculus
agtatactcacgccacccaccgg

SEQ ID NO: 3 - DNA, 23 bp, Mus musculus
tcacgccacccaccggagaatgg

SEQ ID NO: 4 - DNA, 23 bp, Mus musculus
ggcgtatgtatcagtctcagtgg

SEQ ID NO: 5 - DNA, 23 bp, Mus musculus
tcggcttcccattctccggtggg

SEQ ID NO: 6 - DNA, 20 bp, Artificial sequence
  Synthetic
ggagcgcaccatcttcttca

SEQ ID NO: 7 - DNA, 20 bp, Mus musculus
gaggggtttctgagggccac

SEQ ID NO: 8 - DNA, 20 bp, Mus musculus
agtatactcacgccacccac

SEQ ID NO: 9 - DNA, 20 bp, Artificial sequence
  Synthetic
aaaaagtccgcgattacgtc

SEQ ID NO: 10 - DNA, 20 bp, Artificial sequence
  Synthetic
acccatccccgcgtccgaga

SEQ ID NO: 11 - protein, 17 aa, Artificial sequence
  Synthetic
LELGTAWEEVRERFVCD

SEQ ID NO: 12 - protein, 30 aa, Artificial sequence
  Synthetic
LELGTAWEEVRSTAWPQSPSGLRAAELLHH

SEQ ID NO: 13 - protein, 9 aa, Artificial sequence
  Synthetic
ELGTAWEEV

SEQ ID NO: 14 - protein, 9 aa, Artificial sequence
  Synthetic
LGTAWEEVR

SEQ ID NO: 15 - protein, 9 aa, Artificial sequence
  Synthetic
GTAWEEVRS

SEQ ID NO: 16 - protein, 9 aa, Artificial sequence
  Synthetic
TAWEEVRST

SEQ ID NO: 17 - protein, 9 aa, Artificial sequence
  Synthetic
AWEEVRSTA

SEQ ID NO: 18 - protein, 9 aa, Artificial sequence
  Synthetic
WEEVRSTAW

SEQ ID NO: 19 - protein, 9 aa, Artificial sequence
  Synthetic
SGLRAAELL

SEQ ID NO: 20 - protein, 9 aa, Artificial sequence
  Synthetic
GLRAAELLH

SEQ ID NO: 21 - protein, 9 aa, Artificial sequence
  Synthetic
LRAAELLHH

SEQ ID NO: 22 - protein, 10 aa, Artificial sequence
  Synthetic
VNYNFEEEYM

SEQ ID NO: 23 - protein, 8 aa, Artificial sequence
  Synthetic
TAYLFSRF

SEQ ID NO: 24 - protein, 9 aa, Artificial sequence
  Synthetic
SSLQKSSSL

SEQ ID NO: 25 - protein, 8 aa, Artificial sequence
  Synthetic
IVDIFHGL

SEQ ID NO: 26 - protein, 8 aa, Artificial sequence
  Synthetic
KNFAFLEF

SEQ ID NO: 27 - protein, 8 aa, Artificial sequence
  Synthetic
RSFDFIHL

SEQ ID NO: 28 - protein, 8 aa, Artificial sequence
  Synthetic
VAPEFRRL

SEQ ID NO: 29 - protein, 8 aa, Artificial sequence
  Synthetic
RQYVYGVL

SEQ ID NO: 30 - protein, 8 aa, Artificial sequence
  Synthetic
SMIRILGL

SEQ ID NO: 31 - protein, 9 aa, Artificial sequence
  Synthetic
RSWKKLEHL

SEQ ID NO: 32 - protein, 8 aa, Artificial sequence
  Synthetic
SSYAYTLM

SEQ ID NO: 33 - protein, 9 aa, Artificial sequence
  Synthetic
TSPEFSQPL

SEQ ID NO: 34 - protein, 8 aa, Artificial sequence
  Synthetic
IQIMYQNL

SEQ ID NO: 35 - protein, 8 aa, Artificial sequence
  Synthetic
SIYEASSL

SEQ ID NO: 36 - protein, 8 aa, Artificial sequence
  Synthetic
TAYAFHFL

SEQ ID NO: 37 - protein, 12 aa, Artificial sequence
  Synthetic
VNFDFDGAQKKL

SEQ ID NO: 38 - protein, 9 aa, Artificial sequence
  Synthetic
IGQPFYNSL

SEQ ID NO: 39 - protein, 8 aa, Artificial sequence
  Synthetic
FTYVHDEL

SEQ ID NO: 40 - protein, 8 aa, Artificial sequence
  Synthetic
GALVFREV

SEQ ID NO: 41 - protein, 8 aa, Artificial sequence
  Synthetic
SGVGFGGL

SEQ ID NO: 42 - protein, 9 aa, Artificial sequence
  Synthetic
AIVEHLVTL

SEQ ID NO: 43 - protein, 9 aa, Artificial sequence
  Synthetic
SSPVSNVAI

SEQ ID NO: 44 - protein, 9 aa, Artificial sequence
  Synthetic
YSIRHSISM

SEQ ID NO: 45 - protein, 8 aa, Artificial sequence
  Synthetic
YSLQFTNL

SEQ ID NO: 46 - protein, 9 aa, Artificial sequence
  Synthetic
SAVENLNEM

SEQ ID NO: 47 - protein, 9 aa, Artificial sequence
  Synthetic
AMYVHAYTL

SEQ ID NO: 48 - protein, 10 aa, Artificial sequence
  Synthetic
SSYQHTSVTL

SEQ ID NO: 49 - protein, 8 aa, Artificial sequence
  Synthetic
SSFGRGFF

SEQ ID NO: 50 - protein, 9 aa, Artificial sequence
  Synthetic
YQMMLLTKM

SEQ ID NO: 51 - protein, 8 aa, Artificial sequence
  Synthetic
RVLIFSQM

SEQ ID NO: 52 - protein, 9 aa, Artificial sequence
  Synthetic
RSISTCGPL

SEQ ID NO: 53 - protein, 9 aa, Artificial sequence
  Synthetic
GNIKFIGEL

SEQ ID NO: 54 - protein, 9 aa, Artificial sequence
  Synthetic
RFGLNVSSI

SEQ ID NO: 55 - protein, 9 aa, Artificial sequence
  Synthetic
ISPVSSPSL

SEQ ID NO: 56 - protein, 9 aa, Artificial sequence
  Synthetic
KGIMNTTPL

SEQ ID NO: 57 - protein, 8 aa, Artificial sequence
  Synthetic
SNVFVQRL

SEQ ID NO: 58 - protein, 9 aa, Artificial sequence
  Synthetic
ASNINSVII

SEQ ID NO: 59 - protein, 9 aa, Artificial sequence
  Synthetic
KSPLNKTEV

SEQ ID NO: 60 - protein, 9 aa, Artificial sequence
  Synthetic
AAVINKTVV

SEQ ID NO: 61 - protein, 9 aa, Artificial sequence
  Synthetic
VMIPNVETI

SEQ ID NO: 62 - protein, 9 aa, Artificial sequence
  Synthetic
AGIINHGYA

SEQ ID NO: 63 - protein, 9 aa, Artificial sequence
  Synthetic
VALSNMNVI

SEQ ID NO: 64 - protein, 11 aa, Artificial sequence
  Synthetic
QSLVNLRELYL

SEQ ID NO: 65 - protein, 10 aa, Artificial sequence
  Synthetic
KSLINNKNEL

SEQ ID NO: 66 - protein, 9 aa, Artificial sequence
  Synthetic
SQVPNYTLT

SEQ ID NO: 67 - protein, 9 aa, Artificial sequence
  Synthetic
KTIQNLYDL

SEQ ID NO: 68 - protein, 9 aa, Artificial sequence
  Synthetic
SQTYNAEYI

SEQ ID NO: 69 - protein, 9 aa, Artificial sequence
  Synthetic
AALNNHREV

SEQ ID NO: 70 - protein, 9 aa, Artificial sequence
  Synthetic
SSGINVNAA

SEQ ID NO: 71 - protein, 14 aa, Artificial sequence
  Synthetic
KAYTRAVMHFESFI

SEQ ID NO: 72 - protein, 11 aa, Artificial sequence
  Synthetic
TAPSNSVSQSL

SEQ ID NO: 73 - protein, 4 aa, Artificial sequence
  Synthetic
APSG

SEQ ID NO: 74 - protein, 9 aa, Artificial sequence
  Synthetic
SSLNRFSPL

SEQ ID NO: 75 - protein, 4 aa, Artificial sequence
  Synthetic
MKLQ

SEQ ID NO: 76 - protein, 4 aa, Artificial sequence
  Synthetic
TDTL

SEQ ID NO: 77 - protein, 8 aa, Artificial sequence
  Synthetic
CSYKHPVL

SEQ ID NO: 78 - protein, 4 aa, Artificial sequence
  Synthetic
NHFT

SEQ ID NO: 79 - protein, 4 aa, Artificial sequence
  Synthetic
RVSN

SEQ ID NO: 80 - protein, 9 aa, Artificial sequence
  Synthetic
FAALPHAPL

SEQ ID NO: 81 - protein, 10 aa, Artificial sequence
  Synthetic
MSSRLGAVTA

SEQ ID NO: 82 - protein, 9 aa, Artificial sequence
  Synthetic
TPGPTSLKQ

SEQ ID NO: 83 - protein, 15 aa, Artificial sequence
  Synthetic
MSSRLGAVTAVSFLV

SEQ ID NO: 84 - protein, 5 aa, Artificial sequence
  Synthetic
LESQS

SEQ ID NO: 85 - protein, 4 aa, Artificial sequence
  Synthetic
DAVL

SEQ ID NO: 86 - protein, 9 aa, Artificial sequence
  Synthetic
FSNVHSEIL

SEQ ID NO: 87 - protein, 4 aa, Artificial sequence
  Synthetic
KTIQ

SEQ ID NO: 88 - protein, 5 aa, Artificial sequence
  Synthetic
NLYDL

(19) United States
(12) Patent Application Publication
(10) Pub. No.: US 2023/0190707 A1
(43) Pub. Date: Jun. 22, 2023

(54) COMPOSITIONS AND METHODS FOR ENHANCING CANCER IMMUNOTHERAPY

(71) Applicants: FRED HUTCHINSON CANCER CENTER, Seattle, WA (US); Memorial Sloan Kettering Cancer Center, New York, NY (US)

(72) Inventors: Robert K. Bradley, Seattle, WA (US); Omar Abdel-Wahab, New York, NY (US); Sydney Lu, New York, NY (US)

(73) Assignees: Fred Hutchinson Cancer Center, Seattle, WA (US); Memorial Sloan Kettering Cancer Center, New York, NY (US)

(21) Appl. No.: 17/998,906

(22) PCT Filed: May 14, 2021
(86) PCT No.: PCT/US2021/032522

§ 371 Date: Nov. 15, 2022

Related U.S. Application Data
(60) Provisional application No. 63/025,624, filed on May 15, 2020.

(51) Int. Cl. A61K 31/404; A61K 45/06; C07K 16/28

(52) U.S. Cl. CPC A61K 31/404; A61K 45/06; C07K 16/2818; C07K 2317/76

(57) ABSTRACT

The disclosure provides methods of enhancing susceptibility of neoplastic, transformed, and/or cancer cells (“cancer cells”) to immunotherapeutic agents. The methods comprise contacting the cancer cell with an agent that modulates RNA splicing. In some embodiments, the method further comprise contacting the cancer cell with the immunotherapeutic agent, such as an immune checkpoint inhibitor. The disclosure also provides compositions and/or methods for treating a subject with cancer. In some embodiments, the disclosure provides compositions and methods for combination therapy that comprises administering to a subject with cancer an effective amount of an agent that modulates RNA splicing and a therapeutically effective amount of an immunotherapeutic agent, such as an immune checkpoint inhibitor.

### CROSS-REFERENCE TO RELATED APPLICATION

This application claims the benefit of Provisional Application No. 63/025,624, filed May 15, 2020, the disclosure of which is incorporated herein by reference in its entirety

### STATEMENT OF GOVERNMENT LICENSE RIGHTS

This invention was made with Government support under HL128239 and CA251138 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is FHTM174243_Seq_List_FINAL_20210511_ST25.txt. The text file is 16 KB; was created on May 11, 2021; and is being submitted via EFS-Web with the filing of the specification.

### BACKGROUND

Immune checkpoint blockade has greatly improved outcomes of a number of previously difficult-to-manage malignancies. However, many patients do not respond to immune checkpoint blockade. This limitation has spurred intense efforts to identify biomarkers of response, increase response rates, and expand the types of cancers for which immune checkpoint blockade is effective. In this regard, numerous studies have demonstrated that high genomic mutational burden, as well as “mutagenic” genotypes, including mismatch repair deficiency and POLD1/POLE mutations, can be associated with improved clinical outcomes with immune checkpoint blockade. An extensive literature has identified that the correlation between these biomarkers and response to immune checkpoint blockade is believed to occur due to generation of neoantigenic peptides encoded by tumoral mutations.

However, despite the extensive analysis of immune checkpoint blockade therapies and the underlying mechanisms for efficacy or resistance, many patients remain unresponsive to such therapies. Accordingly, there remains a need for therapies to enhance the efficacy of immunotherapeutic strategies to treat hyperproliferative disorders, malignancies and/or cancers. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of enhancing the susceptibility of a cancer cell to an immunotherapeutic agent, comprising contacting the cancer cell with a first agent that modulates RNA splicing.

In one embodiment, the first agent binds and/or inhibits one of the following RNA splicing factors: SF3B1 (SF3b155), SF3B2 (SF3b145), SF3B3 (SF3b130), SF3B4 (SF3b49), SF3B6 (SF3b14a or p14), PHF5A (SF3b14b), SF3B5 (SF3b10), U2AF1 (U2AF35), and U2AF2 (U2AF65). In one embodiment, the first agent is selected from E7107, FD-895, FR901464, H3B-8800, herboxidiene (GEX1A), meayamycin, pladienolide B, pladienolide D, spliceostatin A, isoginkgetin, and madrasin. In one embodiment, the first agent binds, inhibits, and/or degrades via DCAF15 one of the following RNA splicing factors: RBM39 and RBM23. In one embodiment, the first agent causes degradation of RBM39 and/or RBM23. In one embodiment, the first agent is selected from indisulam, E7820, tasisulam, or chloroquinoxaline sulfonamide (CQS). For example, in some embodiments, the first agent is E7820, a compound that degrades RBM39 (see, Faust T. B., et al., (2020.) Structural complementarity facilitates E7820-mediated degradation of RBM39 by DCAF15. Nature Chemical Biology 16, 7-14, incorporated herein by reference in its entirety). In one embodiment, the first agent directly inhibits post-translational modification of one of the following RNA splicing factors: PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, and SART3. In one embodiment, the first agent inhibits one of CLK1, CLK2, CLK3, CLK4, SRPK1, DYRK1a, DYRK1b, a Type I PRMT enzyme, and PRMT5, thereby resulting in inhibition of post-translational modification of the RNA splicing factor. In one embodiment, the Type I PRMT enzyme is selected from PRMT1, PRMT3, PRMT4, PRMT6, and PRMT8. In one embodiment, the first agent inhibits the Type I PRMT enzyme and is selected from MS-023, TC-E 5003, GSK3368715, and the like. In one embodiment, the first agent inhibits PRMT5 and is selected from GSK3326595, EPZ015666, LLY-283, JNJ-64619178, PRT543, and the like.

In some embodiments, the method further comprises contacting the cancer cell with the immunotherapeutic agent or contacting an immune cell with the immunotherapeutic agent and permitting the immune cell to contact the cancer cell.

In some embodiments, the immunotherapeutic agent is a checkpoint inhibitor. In some embodiments, the checkpoint inhibitor targets PD-1, PD-L1, PD-L2, CTLA-4, CD27, CD28, CD40, CD40L, CD122, CD134 (OX40), CD137 (4-1BB), GITR, ICOS, A2AR, CD276 B7-H3), VTCN1 (B7-H4), TMIGD2, BTLA, IDO, NOX2, CD160, LIGHT, LAG3, DNAM-1, TIGIT, CD96, 2B4, Tim-3, SIRPα, CD200R, DR3, LAG3, VISTA, and the like. In some embodiments, the checkpoint inhibitor inhibits PD-1 and is selected from Pembrolizumab (Keytruda), Nivolumab (Opdivo), Cemiplimab (Libtayo), Spartalizumab (PDR001), Camrelizumab (SHR1210), Sintilimab (IB1308), Tislelizumab (BGB-A317), Toripalimab (JS 001), AMP-224, AMP-514, and the like. In some embodiments, the checkpoint inhibitor inhibits PD-L1 and is selected from Atezolizumab (Tecentriq), Avelumab (Bavencio), Durvalumab (Imfinzi), KN035, CK-301, AUNP12, CA-170, BMS-986189, and the like. In some embodiments, the checkpoint inhibitor inhibits CTLA-4 and is selected from Ipilimumab (Yervoy), Tremelimumab, and the like.

In some embodiments, the cancer cell is in vitro.

In some embodiments, the cancer cell is in vivo and contacting the cancer cell comprises administering to the subject a therapeutically effective amount of the agent that modulates RNA splicing. In some embodiments, the method further comprises administering to the subject a therapeutically effective amount of a checkpoint inhibitor as described herein.

In another aspect, the method of treating a cancer in a subject in need thereof. The method comprises administering to the subject a therapeutically effective amount of a first agent that modulates RNA splicing in cancer cells and a therapeutically effective amount of an immunotherapeutic agent.

In some embodiments, the first agent binds and/or inhibits one of the following RNA splicing factors: SF3B1 (SF3b155), SF3B2 (SF3b145), SF3B3 (SF3b130), SF3B4 (SF3b49), SF3B6 (SF3b14a or p14), PHF5A (SF3b14b), SF3B5 (SF3b10), U2AF1 (U2AF35), and U2AF2 (U2AF65). In some embodiments, the first agent is selected from E7107, FD-895, FR901464, H3B-8800, herboxidiene (GEX1A), meayamycin, pladienolide B, pladienolide D, spliceostatin A, isoginkgetin, and madrasin. In some embodiments, the first agent binds, inhibits, and/or degrades via DCAF15 one of the following RNA splicing factors: RBM39 and RBM23. In some embodiments, the first agent causes degradation of RBM39 and/or RBM23. In some embodiments, the first agent is selected from indisulam, E7820, tasisulam, or chloroquinoxaline sulfonamide (CQS). In some embodiments, the first agent directly inhibits post-translational modification of one of the following RNA splicing factors: PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, and SART3. In some embodiments, the first agent inhibits one of CLKT, CLK2, CLK3, CLK4, SRPK1, DYRK1a, DYRK1b, a Type I PRMT enzyme, and PRMT5, thereby resulting in inhibition of post-translational modification of the RNA splicing factor. In some embodiments, the Type I PRMT enzyme is selected from PRMT1, PRMT3, PRMT4, PRMT6, and PRMT8. In some embodiments, the first agent inhibits the Type I PRMT enzymes and is selected from MS-023, TC-E 5003, GSK3368715, and the like. In some embodiments, the first agent inhibits PRMT5 and is selected from GSK3326595, EPZ015666, LLY-283, JNJ-64619178, PRT543, and the like.

In some embodiments, the immunotherapeutic agent is a checkpoint inhibitor. In some embodiments, the checkpoint inhibitor targets PD-1, PD-L1, PD-L2, CTLA-4, CD27, CD28, CD40, CD40L, CD122, CD134 (OX40), CD137 (4-1BB), GITR, ICOS, A2AR, CD276 B7-H3), VTCN1 (B7-H4), TMIGD2, BTLA, IDO, NOX2, CD160, LIGHT, LAG3, DNAM-1, TIGIT, CD96, 2B4, Tim-3, SIRPα, CD200R, DR3, LAG3, VISTA, and the like.

In some embodiments, the checkpoint inhibitor inhibits PD-1 and is selected from Pembrolizumab (Keytruda), Nivolumab (Opdivo), Cemiplimab (Libtayo), Spartalizumab (PDR001), Camrelizumab (SHR1210), Sintilimab (1B1308), Tislelizumab (BGB-A317), Toripalimab (JS 001), AMP-224, AMP-514, and the like. In some embodiments, the checkpoint inhibitor inhibits PD-L1 and is selected from Atezolizumab (Tecentriq), Avelumab (Bavencio), Durvalumab (Imfinzi) KN035, CK-301, AUNP12, CA-170, BMS-986189, and the like. In some embodiments, the checkpoint inhibitor inhibits CTLA-4 and is selected from Ipilimumab (Yervoy), Tremelimumab, and the like. In some embodiments, the agent and the immunotherapeutic agent are administered simultaneously or within a period of 7 days of each other.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1L. Pharmacologic perturbation of RNA splicing impairs tumor growth in a manner dependent on immune recognition. (TA) Schema of drug treatment and subsequent washout followed by ex vivo and in vivo assays. (1B) Western blot of RBM39 in B16-F10 cells following exposure to increasing doses of indisulam over 24 hours (half-maximal inhibitory concentrations, IC₅₀ values, for cell viability as determined by the CellTiter-Glo assay and RBM39 degradation calculated from western blot densitometry are shown) and (1C) 4 days of 1 μM indisulam treatment and then drug washout with recovery of RBM39 protein over time after continued culture in vitro. (TD) Growth of B16-F10 (top row), MC38 (middle), and CT26 cells (bottom row) following 4 days of DMSO or 1 μM indisulam treatment and drug washout in vitro. Curves reflect cell growth after drug washout. Mean±sd shown. (TE) In vivo tumor volumes of the cells from (TD) when engrafted into syngeneic animals. Each line represents an individual tumor (n=10 mice/group, tumors on bilateral flanks). (1F) Box-and-whisker plots of tumor volumes from final day of measurement from (TE). For box and whiskers plots throughout, bar indicates median, box edges first and third quartile values, and whisker edges minimum and maximum values. P-values calculated using Wilcoxon rank-sum test. (1G) Schema of drug treatment followed by engraftment in recipient mice with immune perturbations. (1H) Individual B16-F10 tumor volumes following DMSO or indisulam treatment and then engraftment in C57BL/6 hosts treated with control versus combined CD4 & CD8 T cell depletion. Each line represents an individual tumor (n=10 mice/group; tumors on bilateral flanks). (1I) Box-and-whisker plots of tumor volumes from (H) at day 19; p-values calculated using Wilcoxon rank-sum test: ***, p=0.000379; n.s., p>0.05. (1J) Histograms of cell surface β₂-microglobulin (β₂M; left) or H-2K^{b}/H-2D^{b} (right) on B16-F10 cells edited with control or β₂M sgRNA. (1K) Schema of experiment to evaluate requirement of β₂M for tumor control in vivo after indisulam treatment. (1L) Box-and-whisker plots of individual tumor volumes at day 30 from (1K); p-values calculated using the Wilcoxon rank-sum test: ***, p=0.009; n.s., p>0.05.

FIGS. 2A-2I. Pharmacologic splicing modulation promotes T cell reactivity without causing T cell toxicity in vivo. (2A) In vitro growth of MC38 cells following four days of treatment with DMSO or 5 μM MS-023 for 96 hours and drug washout. Curves reflect cell growth after drug washout. Mean±sd shown. (2B) In vivo growth of cells from (2A) engrafted into syngeneic C57BL/6 mice. Individual tumor volumes are shown (n=10 mice/group; tumors on bilateral flanks). (2C) Violin plots of individual tumor volumes at day 21 from (2B). P-value calculated using Wilcoxon rank-sum test. (2D) Percentage of live CD45.1⁺CD3⁺CD8⁺CFSE^{lo} T cells on day 5 of a mixed leukocyte reaction with syngeneic bone marrow derived dendritic cells from wild-type or β₂-microglobulin knockout C57BL/6 mice, loaded with lysates from MC38 cells treated with the indicated drugs, or overexpressing chicken ovalbumin (ova). Each dot represents a technical replicate. Bar represents median value. The ‘no lysates’ condition indicates T cells incubated with dendritic cells which are not loaded with lysate. The ‘no stimulators’ condition indicates T cells cultured alone, without bone marrow dendritic cells present. P-values calculated using Wilcoxon rank-sum test. For the wild-type donors group, p-values are: DMSO-vs-Ova=0.019, DMSO-vs-indisulam=0.001, DMSO-vs-MS-032=0.032. (2E) Representative histograms of CFSE dilution from (2D). (2F) CFSE labeled CD5⁺ selected naïve splenic T cells from C57BL/6 mice stimulated in plates coated with anti-CD3+CD28 antibodies (10 μg/mL+2 μg/mL) for three days in the presence of indicated concentrations of the labeled drugs. (2G) Wild-type or ovalbumin-expressing B16-F10 cells were cultured alone, or in the presence of activated OT-1 splenic T cells, for 18 hours with splicing drugs at the indicated concentrations. Tumor cells were identified by scatter and CD45⁻ and viability determined by DAPI. (2H) CFSE dilution of donor CD45.1⁺B6 T cells adoptively transferred into lethally irradiated Balb/c recipient animals, treated daily in vivo with the indicated drugs and doses. Splenic CD45.1⁺ CD4⁺ and CD8⁺ T cells on day 3 are shown. (2I) CFSE dilution of donor CD45.1⁺B6 T cells adoptively transferred into lethally irradiated LP/J recipients; splenic CD45.1⁺CD4⁺ and CD8⁺ T cells on day 5 are shown.

FIGS. 4A-4J. Splicing modulation induces widespread potential neoepitope production. (4A) RNA-seq read coverage illustrating shared intron retention (left), cassette exon exclusion (middle), and competing 3′ splice site selection following indisulam treatment of the indicated mouse cancer cell lines. Conditions as in FIG. 1A. (4B) As (4A), but for the indicated human cancer cell lines, treated identically to (4A). (4C) Left, stacked bar graph illustrating numbers of differentially retained introns following indisulam treatment in mouse (top) and human (bottom) cells. Blue/green, increased/decreased intron retention in indisulam relative to DMSO conditions; percentages shown for blue. Right, heat map illustrating quantitative extent of intron retention for introns that are significantly mis-spliced in at least one sample. (4D) As (4C), but for cassette exons. Blue/green, increased/decreased exon skipping in indisulam relative to DMSO conditions. (4E) Bar graph of poly(AT) motif enrichment in introns that were preferentially retained (affected) or whose splicing is unaffected following indisulam treatment. Motif enrichment computed relative to a randomly selected group of unaffected introns. Error bars, 95% confidence intervals estimated by bootstrapping. (4F) Metagene plot illustrating poly(AT) motif enrichment across introns that were preferentially retained (affected) relative to unaffected introns following indisulam treatment in the indicated cell lines. Motif enrichment for introns whose splicing is unaffected by indisulam treatment is also shown (gray line). Shading, 95% confidence intervals estimated by bootstrapping. (4G) Left, RNA-seq read coverage illustrating readily apparent intron retention in the cytoplasmic fraction following indisulam treatment of B16-F10 cells. Right, quantification of Prpf40b intron retention in total, nuclear (nuc.), and cytoplasmic (cyto.) fractions. p computed by unpaired t-test. (4H) Schematic of predicted 9-mer peptides arising from indisulam-induced intron retention in Prp40b. The illustrated sequence for spliced mRNA-derived protein is set forth as SEQ ID NO:11. The illustrated sequences for intro-derived proteins (from top to bottom) are set forth as SEQ ID NOS:12-21. (41) Schematic of the filtering strategy used to predict potential indisulam-induced, MHC I-bound epitopes. Numbers of unique peptides present at each step are shown for representative, common mouse (H-2D^{b}) and human (HLA-A*02:01) alleles following DMSO or indisulam treatment of B16-F10 and 501-MEL cells. (4J) Bar graph illustrating the numbers of predicted indisulam-induced 8-14-mer peptides arising from different types of alternative splicing following DMSO or indisulam treatment of B16-F10 cells. All analyses performed for n=3 biological replicates for each cell line and treatment condition unless specified otherwise.

FIGS. 5A-5Q. Indisulam-induced neopeptides are presented as MHC I-bound epitopes. (5A) Overview of experimental and computational workflow. (5B) Schematic depicting creation of the RNA isoform database and the four proteomes that we analyzed. (5C) Histogram illustrating the predicted binding rank of all peptides identified from the H-2D^{b} immunoprecipitation and full-length proteome. Peptides with rank<2, defined based on NetMHCpan 4.0 predictions, are considered predicted binders. Peptides identified in DMSO-treated (gray, left) and indisulam-treated (red, right) samples are overlaid on a random sample of 1,000 sequences from the full-length proteome (black) for comparison. Data collated across n=3 biological replicates per treatment. (5D) Sequence logo plot of all 9-mers identified from the H-2D^{b} immunoprecipitation and full-length proteome. Y axis, information content in bits. Data collated across n=3 biological replicates per treatment. (5E) Bar plot illustrating numbers of peptides identified from the H-2D^{b} immunoprecipitation using each proteome depicted in (5B). (5F) Bar plot illustrating numbers of predicted binders and non-binders identified from the H-2D^{b} immunoprecipitation using the spiked non-binders proteome, which consists of predicted binders (rank<2), which constitute 90% of this proteome, and non-binders (rank>90), which were added to constitute 10% of this proteome. (5G) Density plots illustrating parent gene expression for peptides identified from the H-2D^{b} immunoprecipitation from DMSO-treated (gray, left) and indisulam-treated (red, right) samples, each compared to the expression of all genes (black) following treatment with DMSO or indisulam, respectively, using the predicted binders proteome. TPM, transcripts per million. Data collated across n=3 biological replicates per treatment. (5H) Heat map illustrating each peptide that was identified from the H-2D^{b} or H-2K^{b} immunoprecipitations in at least one of the three DMSO- and indisulam-treated samples (rows) using the predicted binders proteome. Each column is a peptide. Red, peptides identified exclusively in indisulam-treated samples. (5I) Bar graph illustrating the percentage of indisulam-specific, isoform-specific identified peptides arising from different types of alternative splicing following indisulam treatment of B16-F10 cells. (5J) RNA-seq coverage plots of representative indisulam-induced, candidate splicing-derived neoantigens generated by intron retention events in Hus1 and (5K) Zfp512, (5L) competing 3′ splice sites in D14Abble, and (5M) a cassette exon skipping event in Poldip3. Indisulam-promoted peptide shown in bold, underlined text. (5N) Median fluorescence intensities (MFIs) of H-2D^{b} and/or H-2K^{b} on RMA-S cells following incubation with increasing doses of Hus1, (50) Zfp512, (5P) D14Abble, and (5Q) Poldip3 candidate neoantigenic peptides from (J-M). Mean±sd shown. For (5N-5Q), grey lines indicate negative control peptides that were randomly selected from the predicted non-binder, ‘spike-in’ peptides used in (5B). All analyses performed for n=3 biological replicates for each treatment condition for (5A-5M) and n=4 biological replicates for (5N-5Q).

FIGS. 6A-6G. Splicing-derived neoepitopes are immunogenic in vivo. (6A) Heatmap of mean MFI values of H-2K^{b} expression from RMA-S peptide stabilization experiments across 40 peptides with H-2K^{b} binding from RMA-S assay. Highlighted text indicates control known immunogenic peptides (SIINFEKL (SEQ ID NO:1) and Trp1 heteroclitic peptide). The sequence identifiers (i.e., SEQ ID NOS) for the remaining peptides sequences represented are indicated in parentheses. (6B) Schema of hock immunization of C57BL/6 mice with individual peptides emulsified in TiterMax. (6C) Representative IFNγ ELISpot data from CD8⁺ T cells harvested from draining lymph nodes following stimulation with syngeneic peptide-loaded splenocytes. Each row represents data for a single peptide (including SIINFEKL (SEQ ID NO: 1)) used in in vivo immunization. Each column indicates T cells reacted with the indicated stimuli. PMA: Phorbol 12-myristate 13-acetate; Iono: ionomycin. (6D) Number of spots per 10⁵ CD8⁺ T cells from IFNγ ELISpot quantified for the peptides identified as immunogenic in vivo from the intersection of RNA-seq and mass spectrometry analyses. Cognate peptides used for immunization and stimulation for IFNγ response shown on x-axis. Bar indicates median. SIINFEKL (SEQ ID NO:1), highlighted, shown as positive control. The sequence identifiers (SEQ ID NOS) are indicated in parentheses. Each dot represents a technical replicate. (6E) Representative IFNγ ELISpot data from CD8⁺ T cells harvested from draining lymph nodes of mice immunized with 0.1, 1, 10 or 100 μg of the indicated peptide, following stimulation with syngeneic peptide-loaded splenocytes. Each row indicates one peptide dose. Each column indicates T cells reacted with the indicated stimuli. Plots on right quantify number of dots per well, with each dot representing one well (technical replicate). SIINFEKL (SEQ ID NO: 1) as positive control is indicated. (6F) Comparisons of predicted MHC I binding for immunogenic (IFNγ ELISpot-positive) versus nonimmunogenic (ELISpot negative) peptides. (6G) As (6F), but illustrates experimentally determined MFI values (from (6A)). For (6F-6G), P-values were computed using the two-sided Wilcoxon rank-sum test.

FIGS. 7A-7K. Splicing-derived neoantigens trigger an endogenous T cell response. (7A) Schema of CD8⁺ T cells from peptide-immunized C57BL/6 mice, co-cultured for 72 hours with B16-F10 loaded with peptides, to assess for cytotoxicity. (7B) Quantification of live B16-F10 cells from (7A) after co-culture. CD8⁺ T cells were obtained from mice immunized with the peptide indicated below each horizontal line (including SIINFEKL (SEQ ID NO:1)), and reacted with B16-F10 loaded with peptides (including SIINFEKL (SEQ ID NO:1)) as indicated by the x-axis labels. Each dot indicates a technical replicate. P-values were calculated with the Wilcoxon rank-sum test. (7C) Schema of CD8⁺ T cells from peptide-immunized C57BL/6 mice, stimulated with B16-F10 treated with DMSO or indisulam as antigen presenting cells, for IFNγ ELISpot. (7D) Representative IFNγ ELISpot data from CD8⁺ T cells harvested from draining lymph nodes following stimulation with DMSO or indisulam-treated B16-F10 cells, or B16-F10 cells overexpressing ovalbumin. Each row indicates one peptide used to immunize C57BL/6 mice to generate CD8⁺ T cells (including SIINFEKL (SEQ ID NO:1)). Each column indicates T cells reacted with the indicated type of B16-F10 tumor. PMA: Phorbol 12-myristate 13-acetate; Iono: ionomycin. (7E) Bubble plot quantification of data from (7C-7D); the number of spots in each condition is represented by the size of the bubble. Grey color indicates no statistical significance, orange p<0.05 and red p<0.01 by Wilcoxon rank-sum test. (7F-7H) Box-and-whisker plots visualizing representative peptides from (7E), i.e., SIINFEKL (SEQ ID NO:1), SQVPNYTLT (SEQ ID NO:66), and TAYAFHFL (SEQ ID NO:36), respectively. Each dot represents a technical replicate (ELISpot well). P-values were calculated using the Wilcoxon rank-sum test. (7I) RNA-seq coverage plots demonstrating mis-splicing of the Eif4g3 (left) and Stat2 (right) genes upon indisulam exposure, as well as the resultant neoantigenic peptide. The sequence identifiers for the illustrated peptide sequences are as follows: APSG (SEQ ID NO:73), SSLNRFSPL (SEQ ID NO:74), MKLQ (SEQ ID NO:75), TDTL (SEQ ID NO:76), and CSYKHPVL (SEQ ID NO:77). (7J) Representative contour plots of peptide:MHC I tetramer staining of CD8⁺ T cells from the tumor draining lymph nodes of B16-F10 tumor-bearing mice treated with vehicle, anti-PD1, indisulam, or the combination and analyzed at day 14, gated on CD3⁺ T cells. Each row indicates one neoantigenic peptide, and each column indicates a treatment condition. (7K) Quantification of results from (7J); each dot represents one biological replicate (mouse). P-values were calculated using the Kruskal-Wallis nonparametric ANOVA.

FIGS. 8A-8D. Indisulam induces dose-dependent splicing alterations which are associated with dose-dependent effects on tumor growth in vivo. MC38 or CT26 cells were treated with DMSO or indisulam at concentrations of 10, 100, or 1000 nM for 96 hours in technical triplicate; these were then subjected to RNA-seq analyses or used for biological experiments. (8A) Total number of splicing alterations in CT26 and MC38 tumors exposed to the indicated doses of indisulam, as compared with DMSO. Next, the CT26 tumors treated with indisulam in vitro were engrafted into syngeneic Balb/c mice. (8B) Tumor volumes of CT26 bearing mice over time (n=15 mice/group; tumors engrafted on bilateral flanks of mice). Mean±sem. (8C) tumor volumes from (8B) at day 24. P-values were calculated for the indicated group compared to DMSO control using the Wilcoxon rank-sumtest: *, p=0.048; ***, p=0.000798; ****, p=0.000363. (8D) Kaplan-Meier survival curve of animals.

FIGS. 9A and 9B. Treatment of tumor bearing animals with splicing modulator compounds in vivo enhances tumor control and can elicit memory. MC38 cells were treated with DMSO or MS-023 in vitro for 96 hours, and then 10⁶ cells engrafted onto the bilateral flanks of either naïve C57BL/6 mice, or animals which had successfully rejected MC38 tumors previously after treatment with anti-PD1 and MS-023 in vivo (from FIGS. 3K-3M). (9A) Line graphs summarizing growth curves of individual tumors from engrafted mice showing mean±sem. (9B) Violin plots showing tumor volumes at day 28. P-values (calculated using the Wilcoxon rank-sum test) for the “DMSO survivor”-vs “DMSO naïve” and “MS-023 survivor”-vs-“MS-023 naïve” comparisons are 0.044 and 0.011, respectively.

### DETAILED DESCRIPTION

Due to the observed practical limitations of current immune checkpoint inhibitor therapies, many efforts have been made to identify biomarkers indicating the response to checkpoint blockade as well as pharmacologic approaches to increase response to these therapies. Mutations in DNA are the best-studied source of neoantigens that determine response to immune checkpoint blockade. The present disclosure is based on the inventors' analysis of neoantigens resulting not from genetic alterations but rather from alterations in RNA splicing within cancer cells. In view of the paucity of functional evidence that increased RNA splicing lead to neoantigens in cancer cells, the inventors surprisingly found that pharmacologic perturbation of RNA splicing via multiple, distinct drug classes generates bona fide neoantigens and elicits anti-tumor immunity, augmenting checkpoint immunotherapy. This resulted in an anti-tumor immune response in vivo leading to, e.g., reduced tumor volume. Further, the inventors demonstrated that splicing modulation inhibited tumor growth and enhanced checkpoint blockade in a manner dependent on host T cells and peptides presented on tumor MHC class I. Significantly, splicing modulation induced stereotyped splicing changes across tumor types, altering the MHC I-bound immunopeptidome to yield splicing-derived neoepitopes that trigger an anti-tumor T cell response in vivo. These data definitively identify splicing modulation as an untapped source of immunogenic peptides and provide a useful strategy to enhance response to checkpoint blockade that is readily translatable to the clinic across cancer types.

In accordance with the foregoing, in one aspect the disclosure provides a method of enhancing the susceptibility of a cancer cell to an immunotherapeutic agent. The method comprises contacting the cancer cell with a first agent that modulates RNA splicing.

As indicated above and described in more detail below, the inventors demonstrated that induced perturbations in RNA splicing leads to neoantigen production, increased anti-tumor response, and greater sensitivity to checkpoint inhibitor therapeutics across a variety of distinct cancer cell types. Accordingly, the disclosure is not limited to any particular cancer or cancer cell types but is applicable to and encompasses cancers and/or cancer cells generally. As used herein, the term “cancer” is used generally to refer diseases characterized by abnormal cell growth, division, and/or development, including neoplasms, benign tumor growths, dysplastic diseases, hyperproliferative disorders, and malignancies. The term “cancer cell” is used generally to refer to any transformed cell where one or more genetic mutations lead to dysregulation of (e.g., loss of an aspect of control over) cell growth, development, and/or cell-cycle compared to the healthy cells originating from the same tissue. The cancer cells typically exhibit unique gene expression patterns and phenotypes compared to their healthy cell counterparts, including increased and uncontrolled cell growth, uncontrolled cell division, altered (e.g., abnormal) cell development or differentiation. The cells can be a neoplastic cell, a precancerous cell, benign tumor cells, or malignant neoplasm (malignant cancer) cell. For example, consequent to a loss of normal cell-cycle regulation, the cancer cell can develop and/or proliferate at an enhanced rate, thus potentially giving rise to a cell-proliferative disease, such as malignant or benign cancers.

Broadly, cancer cells encompassed by the disclosure can be categorized by the type of cell that is presumed to be origin of the cancer cell (e.g., carcinomas from epithelial cells, sarcomas from connective tissue cells, myelodysplastic syndromes (MDS), lymphomas and leukemias from hematopoietic cells, blastomas from immature precursor cells and embryonic tissues, etc.) Cancers (or cancer cells) discussed herein can be any type of neoplasms, benign tumor growths, dysplastic diseases, hyperproliferative disorders, and malignancies (or cell thereof). For example, the cancer cell can be, e.g., a cell characteristic of a myelodysplastic syndrome (MDS), which is often considered a pre- (malignant) cancer. In other embodiments, the cancer cell can be a metastatic cancer cell. The cancer cells can be in or derived from solid or non-solid tumors. Cancers and cancer cell types contemplated herein include, but are not limited to: adrenal cancer, anal cancer, bladder cancer, blood cancer, bone cancer, brain cancer, breast cancer, cervical cancer, chronic or acute leukemia, CNS cancer, colon cancer, cutaneous or intraocular or mucosal melanoma, endocrine cancer, endometrial carcinoma, esophageal cancer, fallopian tube carcinoma, follicular lymphoma and other non-Hodgkin's lymphomas, gastric cancer, head or neck cancer, Hodgkin's disease, kidney cancer, larynx cancer, large intestinal cancer, liver cancer, lung cancer, lymphocytic lymphoma, ovarian cancer, pancreatic cancer, parathyroid cancer, penile cancer, pituitary adenoma, primary CNS lymphoma, prostate cancer, neuroendocrine cancers, rectal cancer, renal cancer (e.g., renal cell carcinoma and renal pelvic cancer), skin cancer, small cell lung cancer, small intestinal cancer, soft tissue tumor, spleen cancer, stomach cancer, testicular cancer, thyroid cancer, ureter cancer, urethral cancer, uterine cancer, vaginal cancer, and vulval cancer, or a combination thereof. Specific, exemplary cancers include without limitation: adrenocortical carcinoma (ACC), bladder urothelial cancer (BLCA), breast invasive carcinoma (BRCA), cervical squamous cell carcinoma and endocervical adenocarcinoma (CESC), cholangiocarcinoma (CHOL), colon adenocarcinoma (COAD), colorectal adenocarcinoma (COAD/READ), lymphoid neoplasm diffuse B-cell lymphoma (DLBC), esophageal carcinoma (ESCA), head & neck squamous carcinoma (HNSC), kidney chromophobe (KICH), kidney renal clear cell carcinoma (KIRC), kidney renal papillary cell carcinoma (KIRP), acute myeloid leukemia (LAML), liver hepatocellular carcinoma (LIHC), lung adenocarcinoma (LUAD), lung squamous cell carcinoma (LUSC), mesothelioma (MES), myelodysplastic syndrome (MDS), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), stomach adenocarcinoma (STAD), stomach and esophageal (STES), testicular germ cell tumor (TGCT), thyroid carcinoma (THCA), thymoma (THYM), uterine corpus endometrial carcinoma (UCEC), uterine carcinoma (UCS), uveal melanoma (UVM), and gliomas such as low grade glioma (LGG) and glioblastoma (GBM), each of which is encompassed by the present disclosure. Furthermore, the cancer can be a pediatric cancer such as Wilms tumor (WT), rhabdoid tumor (RT), neuroblastoma (NBL), and clear cell sarcoma of the kidney (CCSK).

The term “enhancing the susceptibility of a cancer cell to an immunotherapeutic agent” refers to increasing the likelihood cancer cell inhibition or killing by, or decreasing resistance of the cancer cell to, the immunotherapeutic agent. The phrase encompasses direct and indirect activity of the immunotherapeutic agent on the cancer cell. An example of an indirect activity of the immunotherapeutic agent encompassed by the present application is an immunotherapeutic agent that enhances or modulates activity of an immune cell (e.g., a T cell or B cell), after which the immune cell has enhanced activity against the cancer cell. An enhanced susceptibility can be established by exposing the cancer cell contacted with the first agent to the immunotherapeutic agent, or the components required in an indirect interaction (e.g., immunotherapeutic agent-exposed immune cell), and comparing a parameter of cancer cell health, activity, or viability (e.g., cell death or motility, etc.) against a control cell of the same type that is not contacted with the first agent. More description of immunotherapeutic agents with direct or indirect interactions with the cancer cell is provided below.

The first agent in the method can be any agent that modulates RNA splicing. In some embodiments, the first agent can be defined by the ability to bind and/or inhibit an RNA splicing factor. Exemplary first agents encompassed by the disclosure can be characterized as binding to and/or inhibiting one of the following RNA splicing factors: SF3B1 (SF3b155), SF3B2 (SF3b145), SF3B3 (SF3b30), SF3B4 (SF3b49), SF3B6 (SF3b14a or p14), PHF5A (SF3b14b), SF3B5 (SF3b10), U2AF1 (U2AF35), and U2AF2 (U2AF65). Many such agents are known, such as E7107, FD-895, FR901464, H3B-8800, herboxidiene (GEX1A), meayamycin, pladienolide B, pladienolide D, spliceostatin A, isoginkgetin, and madrasin, each of which is encompassed by the present disclosure as an embodiments of the first agent.

In some embodiments, the first agent binds, inhibits, and/or otherwise degrades one of the following RNA splicing factors: RBM39 and RBM23. In some embodiments, the inhibition can occur via interaction with via DCAF15. Exemplary agents with this functionality are known, including indisulam, E7820, tasisulam, and chloroquinoxaline sulfonamide (CQS), each of which are encompassed by this disclosure. In some embodiments, the first agent causes degradation of RBM39 and/or RBM23. In some embodiments, the first agent is indisulam, E7820, tasisulam, or chloroquinoxaline sulfonamide (CQS).

In some embodiments, the first agent indirectly inhibits post-translational modification of one of the following RNA splicing factors: PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, and SART3. In some embodiments, the first agent inhibits one of CLK1, CLK2, CLK3, CLK4, SRPK1, DYRK1a, DYRK1b, a Type I PRMT enzyme (such as PRMT1, PRMT3, PRMT4, PRMT6, PRMT8), and PRMT5, thereby resulting in inhibition of post-translational modification of the RNA splicing factor. Agents that inhibit post-translational modification of the described RNA splicing factors are known and encompassed by this disclosure. For example, in some embodiments, the first agent inhibits Type I PRMT enzymes (such as PRMT1, PRMT3, PRMT4, PRMT6, PRMT8) and is selected from MS-023, TC-E 5003, GSK3368715, and the like. In some embodiments, the first agent inhibits PRMT5 and is selected from GSK3326595, EPZ015666, LLY-283, JNJ-64619178, PRT543, and the like.

As described herein, induced modulation of RNA splicing, e.g., by inhibition or interruption of normal RNA splicing regulation mechanisms by RNA splicing factors, results in production of neoantigens in the target cancer cells. The inventors demonstrated that the enhanced neo-antigen production sensitizes the cancer cell to immunotherapies, resulting in synergistic effects. Accordingly, in some embodiments, the method further comprises contacting the cancer cell with the immunotherapeutic agent. Immunotherapeutic agent can include, e.g., antibodies, immune cells, cytokines, etc., which can boost response of an immune systems (e.g., a subject's own immune response), or isolated immune system component (e.g., an isolated lymphocyte), against the cancer target. Such immunotherapeutic agents include adoptive immune cell therapies, including chimeric antigen receptor engineered T cells (CAR T-cells), engineered T-Cell Receptor (TCR) T cells, immune checkpoint inhibitor therapies, cancer vaccines, and the like. In these embodiments, the immunotherapeutic agent acts directly on the target cell. In other embodiments, the method further comprises contacting an immune cell with the immunotherapeutic agent and permitting the immune cell to contact the cancer cell. In these embodiments, the immunotherapeutic agent acts indirectly on the target cell by enhancing the functionality of the immune cell in a manner to increase the immune cell's ability to kill or otherwise inhibit the growth of the target cancer cell.

In some embodiments, the immunotherapeutic agent is an immune system checkpoint inhibitor. Broadly described, checkpoint inhibitors are agents that counteract cancer cells' signaling mechanisms that would normally attack and block stimulating checkpoint targets on the immune cell to prevent a responsive phenotype. The checkpoint inhibitor agents, interrupt this interaction between the cancer cell and the immune cell, thereby restoring stimulatory signaling in the immune cells. In some embodiments, the checkpoint inhibitor specifically binds a target (e.g., receptor or ligand) on the immune cells to block or outcompete interaction by a corresponding signaling factor expressed on the cancer cell. In some embodiments, the checkpoint inhibitor specifically binds a target (e.g., receptor or ligand) on the cancer cell to block or outcompete interaction by a corresponding signaling factor (e.g., receptor or ligand) expressed on an immune cell. In some embodiments, the checkpoint inhibitor targets PD-1, PD-L1, PD-L2, CTLA-4, CD27, CD28, CD40, CD40L, CD122, CD134 (OX40), CD137 (4-1BB), GITR, ICOS, A2AR, CD276 B7-H3), VTCN1 (B7-H4), TMIGD2, BTLA, IDO, NOX2, CD160, LIGHT, LAG3, DNAM-1, TIGIT, CD96, 2B4, Tim-3, SIRPα, CD200R, DR3, LAG3, VISTA, and the like. Checkpoint inhibitors targeting these factors are known and encompassed by the present disclosure. To illustrate, in some exemplary and non-limiting embodiments, the checkpoint inhibitor inhibits PD-1 and can be selected from Pembrolizumab (Keytruda), Nivolumab (Opdivo), Cemiplimab (Libtayo), Spartalizumab (PDR001), Camrelizumab (SHR1210), Sintilimab (IB1308), Tislelizumab (BGB-A317), Toripalimab (JS 001), AMP-224, AMP-514, and the like. In other exemplary embodiments, the checkpoint inhibitor inhibits PD-L1 and is selected from Atezolizumab (Tecentriq), Avelumab (Bavencio), Durvalumab (Imfinzi), KN035, CK-301, AUNP12, CA-170, BMS-986189, and the like. In some embodiments, the checkpoint inhibitor inhibits CTLA-4 and is selected from Ipilimumab (Yervoy), Tremelimumab, and the like.

The inventors established that modulation of RNA splicing by mechanistically different approaches confers sensitivity to immune checkpoint therapies in general. Accordingly, the described methods are not limited to specific combinations of RNA-splicing modulating agents (i.e., “first agent”) and immunotherapeutic agent, but instead the present disclosure encompasses any combination of first agent (i.e., an agent that modulates RNA splicing, as described above) and immunotherapeutic agent (e.g., an immune checkpoint inhibitor) as described above. For example, the method encompasses any combination of a first agent that targets (e.g., binds to and/or inhibits) SF3B1 (SF3b155), SF3B2 (SF3b145), SF3B3 (SF3b130), SF3B4 (SF3b49), SF3B6 (SF3b14a or p14), PHF5A (SF3b14b), SF3B5 (SF3b10), U2AF1 (U2AF35), U2AF2 (U2AF65), RBM39, or RBM23, or inhibits post-translational modification of PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, SART3, CLK1, CLK2, CLK3, CLK4, SRPK1, DYRK1a, DYRK1b, a Type I PRMT enzyme (such as PRMT1, PRMT3, PRMT4, PRMT6, PRMT8), or PRMT5, with a checkpoint inhibitor.

For purposes of illustration, in some illustrative embodiments the method comprises contacting the cancer cell with one or the following combinations of first agent and immunotherapeutic agent: E7107 and Pembrolizumab (Keytruda), E7107 and Nivolumab (Opdivo), E7107 and Cemiplimab (Libtayo), E7107 and Spartalizumab (PDR001), E7107 and Camrelizumab (SHR1210), E7107 and Sintilimab (IBI308), E7107 and Tislelizumab (BGB-A317), E7107 and Toripalimab (JS 001), E7107 and AMP-224, E7107 and AMP-514, E7107 and Atezolizumab (Tecentriq), E7107 and Avelumab (Bavencio), E7107 and Durvalumab (Imfinzi), E7107 and KN035, E7107 and CK-301, E7107 and AUNP12, E7107 and CA-170, E7107 and BMS-986189, E7107 and Ipilimumab (Yervoy), E7107 and Tremelimumab, FD-895 and Pembrolizumab (Keytruda), FD-895 and Nivolumab (Opdivo), FD-895 and Cemiplimab (Libtayo), FD-895 and Spartalizumab (PDR001), FD-895 and Camrelizumab (SHR1210), FD-895 and Sintilimab (IBI308), FD-895 and Tislelizumab (BGB-A317), FD-895 and Toripalimab (JS 001), FD-895 and AMP-224, FD-895 and AMP-514, FD-895 and Atezolizumab (Tecentriq), FD-895 and Avelumab (Bavencio), FD-895 and Durvalumab (Imfinzi), FD-895 and KN035, FD-895 and CK-301, FD-895 and AUNP12, FD-895 and CA-170, FD-895 and BMS-986189, FD-895 and Ipilimumab (Yervoy), FD-895 and Tremelimumab, FR901464 and Pembrolizumab (Keytruda), FR901464 and Nivolumab (Opdivo), FR901464 and Cemiplimab (Libtayo), FR901464 and Spartalizumab (PDR001), FR901464 and Camrelizumab (SHR1210), FR901464 and Sintilimab (IBI308), FR901464 and Tislelizumab (BGB-A317), FR901464 and Toripalimab (JS 001), FR901464 and AMP-224, FR901464 and AMP-514, FR901464 and Atezolizumab (Tecentriq), FR901464 and Avelumab (Bavencio), FR901464 and Durvalumab (Imfinzi), FR901464 and KN035, FR901464 and CK-301, FR901464 and AUNP12, FR901464 and CA-170, FR901464 and BMS-986189, FR901464 and Ipilimumab (Yervoy), FR901464 and Tremelimumab, H3B-8800 and Pembrolizumab (Keytruda), H3B-8800 and Nivolumab (Opdivo), H3B-8800 and Cemiplimab (Libtayo), H3B-8800 and Spartalizumab (PDR001), H3B-8800 and Camrelizumab (SHR1210), H3B-8800 and Sintilimab (IBI308), H3B-8800 and Tislelizumab (BGB-A317), H3B-8800 and Toripalimab (JS 001), H3B-8800 and AMP-224, H3B-8800 and AMP-514, H3B-8800 and Atezolizumab (Tecentriq), H3B-8800 and Avelumab (Bavencio), H3B-8800 and Durvalumab (Imfinzi), H3B-8800 and KN035, H3B-8800 and CK-301, H3B-8800 and AUNP12, H3B-8800 and CA-170, H3B-8800 and BMS-986189, H3B-8800 and Ipilimumab (Yervoy), H3B-8800 and Tremelimumab, herboxidiene (GEX1A) and Pembrolizumab (Keytruda), herboxidiene (GEX1A) and Nivolumab (Opdivo), herboxidiene (GEX1A) and Cemiplimab (Libtayo), herboxidiene (GEX1A) and Spartalizumab (PDR001), herboxidiene (GEX1A) and Camrelizumab (SHR1210), herboxidiene (GEX1A) and Sintilimab (IBI308), herboxidiene (GEX1A) and Tislelizumab (BGB-A317), herboxidiene (GEX1A) and Toripalimab (JS 001), herboxidiene (GEX1A) and AMP-224, herboxidiene (GEX1A) and AMP-514, herboxidiene (GEX1A) and Atezolizumab (Tecentriq), herboxidiene (GEX1A) and Avelumab (Bavencio), herboxidiene (GEX1A) and Durvalumab (Imfinzi), herboxidiene (GEX1A) and KN035, herboxidiene (GEX1A) and CK-301, herboxidiene (GEX1A) and AUNP12, herboxidiene (GEX1A) and CA-170, herboxidiene (GEX1A) and BMS-986189, herboxidiene (GEX1A) and Ipilimumab (Yervoy), herboxidiene (GEX1A) and Tremelimumab, meayamycin and Pembrolizumab (Keytruda), meayamycin and Nivolumab (Opdivo), meayamycin and Cemiplimab (Libtayo), meayamycin and Spartalizumab (PDR001), meayamycin and Camrelizumab (SHR1210), meayamycin and Sintilimab (IBI308), meayamycin and Tislelizumab (BGB-A317), meayamycin and Toripalimab (JS 001), meayamycin and AMP-224, meayamycin and AMP-514, meayamycin and Atezolizumab (Tecentriq), meayamycin and Avelumab (Bavencio), meayamycin and Durvalumab (Imfinzi), meayamycin and KN035, meayamycin and CK-301, meayamycin and AUNP12, meayamycin and CA-170, meayamycin and BMS-986189, meayamycin and Ipilimumab (Yervoy), meayamycin and Tremelimumab, pladienolide B and Pembrolizumab (Keytruda), pladienolide B and Nivolumab (Opdivo), pladienolide B and Cemiplimab (Libtayo), pladienolide B and Spartalizumab (PDR001), pladienolide B and Camrelizumab (SHR1210), pladienolide B and Sintilimab (IBI308), pladienolide B and Tislelizumab (BGB-A317), pladienolide B and Toripalimab (JS 001), pladienolide B and AMP-224, pladienolide B and AMP-514, pladienolide B and Atezolizumab (Tecentriq), pladienolide B and Avelumab (Bavencio), pladienolide B and Durvalumab (Imfinzi), pladienolide B and KN035, pladienolide B and CK-301, pladienolide B and AUNP12, pladienolide B and CA-170, pladienolide B and BMS-986189, pladienolide B and Ipilimumab (Yervoy), pladienolide B and Tremelimumab, pladienolide D and Pembrolizumab (Keytruda), pladienolide D and Nivolumab (Opdivo), pladienolide D and Cemiplimab (Libtayo), pladienolide D and Spartalizumab (PDR001), pladienolide D and Camrelizumab (SHR1210), pladienolide D and Sintilimab (IBI308), pladienolide D and Tislelizumab (BGB-A317), pladienolide D and Toripalimab (JS 001), pladienolide D and AMP-224, pladienolide D and AMP-514, pladienolide D and Atezolizumab (Tecentriq), pladienolide D and Avelumab (Bavencio), pladienolide D and Durvalumab (Imfinzi), pladienolide D and KN035, pladienolide D and CK-301, pladienolide D and AUNP12, pladienolide D and CA-170, pladienolide D and BMS-986189, pladienolide D and Ipilimumab (Yervoy), pladienolide D and Tremelimumab, spliceostatin A and Pembrolizumab (Keytruda), spliceostatin A and Nivolumab (Opdivo), spliceostatin A and Cemiplimab (Libtayo), spliceostatin A and Spartalizumab (PDR001), spliceostatin A and Camrelizumab (SHR1210), spliceostatin A and Sintilimab (IBI308), spliceostatin A and Tislelizumab (BGB-A317), spliceostatin A and Toripalimab (JS 001), spliceostatin A and AMP-224, spliceostatin A and AMP-514, spliceostatin A and Atezolizumab (Tecentriq), spliceostatin A and Avelumab (Bavencio), spliceostatin A and Durvalumab (Imfinzi), spliceostatin A and KN035, spliceostatin A and CK-301, spliceostatin A and AUNP12, spliceostatin A and CA-170, spliceostatin A and BMS-986189, spliceostatin A and Ipilimumab (Yervoy), spliceostatin A and Tremelimumab, isoginkgetin and Pembrolizumab (Keytruda), isoginkgetin and Nivolumab (Opdivo), isoginkgetin and Cemiplimab (Libtayo), isoginkgetin and Spartalizumab (PDR001), isoginkgetin and Camrelizumab (SHR1210), isoginkgetin and Sintilimab (IBI308), isoginkgetin and Tislelizumab (BGB-A317), isoginkgetin and Toripalimab (JS 001), isoginkgetin and AMP-224, isoginkgetin and AMP-514, isoginkgetin and Atezolizumab (Tecentriq), isoginkgetin and Avelumab (Bavencio), isoginkgetin and Durvalumab (Imfinzi), isoginkgetin and KN035, isoginkgetin and CK-301, isoginkgetin and AUNP12, isoginkgetin and CA-170, isoginkgetin and BMS-986189, isoginkgetin and Ipilimumab (Yervoy), isoginkgetin and Tremelimumab, madrasin and Pembrolizumab (Keytruda), madrasin and Nivolumab (Opdivo), madrasin and Cemiplimab (Libtayo), madrasin and Spartalizumab (PDR001), madrasin and Camrelizumab (SHR1210), madrasin and Sintilimab (IBI308), madrasin and Tislelizumab (BGB-A317), madrasin and Toripalimab 30 (JS 001), madrasin and AMP-224, madrasin and AMP-514, madrasin and Atezolizumab (Tecentriq), madrasin and Avelumab (Bavencio), madrasin and Durvalumab (Imfinzi), madrasin and KN035, madrasin and CK-301, madrasin and AUNP12, madrasin and CA-170, madrasin and BMS-986189, madrasin and Ipilimumab (Yervoy), madrasin and Tremelimumab, indisulam and Pembrolizumab (Keytruda), indisulam and Nivolumab (Opdivo), indisulam and Cemiplimab (Libtayo), indisulam and Spartalizumab (PDR001), indisulam and Camrelizumab (SHR1210), indisulam and Sintilimab (IBI308), indisulam and Tislelizumab (BGB-A317), indisulam and Toripalimab (JS 001), indisulam and AMP-224, indisulam and AMP-514, indisulam and Atezolizumab (Tecentriq), indisulam and Avelumab (Bavencio), indisulam and Durvalumab (Imfinzi), indisulam and KN035, indisulam and CK-301, indisulam and AUNP12, indisulam and CA-170, indisulam and BMS-986189, indisulam and Ipilimumab (Yervoy), indisulam and Tremelimumab, E7820 and Pembrolizumab (Keytruda), E7820 and Nivolumab (Opdivo), E7820 and Cemiplimab (Libtayo), E7820 and Spartalizumab (PDR001), E7820 and Camrelizumab (SHR1210), E7820 and Sintilimab (IBI308), E7820 and Tislelizumab (BGB-A317), E7820 and Toripalimab (JS 001), E7820 and AMP-224, E7820 and AMP-514, E7820 and Atezolizumab (Tecentriq), E7820 and Avelumab (Bavencio), E7820 and Durvalumab (Imfinzi), E7820 and KN035, E7820 and CK-301, E7820 and AUNP12, E7820 and CA-170, E7820 and BMS-986189, E7820 and Ipilimumab (Yervoy), E7820 and Tremelimumab, tasisulam and Pembrolizumab (Keytruda), tasisulam and Nivolumab (Opdivo), tasisulam and Cemiplimab (Libtayo), tasisulam and Spartalizumab (PDR001), tasisulam and Camrelizumab (SHR1210), tasisulam and Sintilimab (IBI308), tasisulam and Tislelizumab (BGB-A317), tasisulam and Toripalimab (JS 001), tasisulam and AMP-224, tasisulam and AMP-514, tasisulam and Atezolizumab (Tecentriq), tasisulam and Avelumab (Bavencio), tasisulam and Durvalumab (Imfinzi), tasisulam and KN035, tasisulam and CK-301, tasisulam and AUNP12, tasisulam and CA-170, tasisulam and BMS-986189, tasisulam and Ipilimumab (Yervoy), tasisulam and Tremelimumab, chloroquinoxaline sulfonamide (CQS) and Pembrolizumab (Keytruda), chloroquinoxaline sulfonamide (CQS) and Nivolumab (Opdivo), chloroquinoxaline sulfonamide (CQS) and Cemiplimab (Libtayo), chloroquinoxaline sulfonamide (CQS) and Spartalizumab (PDR001), chloroquinoxaline sulfonamide (CQS) and Camrelizumab (SHR1210), chloroquinoxaline sulfonamide (CQS) and Sintilimab (IBI308), chloroquinoxaline sulfonamide (CQS) and Tislelizumab (BGB-A317), chloroquinoxaline sulfonamide (CQS) 30 and Toripalimab (JS 001), chloroquinoxaline sulfonamide (CQS) and AMP-224, chloroquinoxaline sulfonamide (CQS) and AMP-514, chloroquinoxaline sulfonamide (CQS) and Atezolizumab (Tecentriq), chloroquinoxaline sulfonamide (CQS) and Avelumab (Bavencio), chloroquinoxaline sulfonamide (CQS) and Durvalumab (Imfinzi), chloroquinoxaline sulfonamide (CQS) and KN035, chloroquinoxaline sulfonamide (CQS) and CK-301, chloroquinoxaline sulfonamide (CQS) and AUNP12, chloroquinoxaline sulfonamide (CQS) and CA-170, chloroquinoxaline sulfonamide (CQS) and BMS-986189, chloroquinoxaline sulfonamide (CQS) and Ipilimumab (Yervoy), chloroquinoxaline sulfonamide (CQS) and Tremelimumab, MS-023 and Pembrolizumab (Keytruda), MS-023 and Nivolumab (Opdivo), MS-023 and Cemiplimab (Libtayo), MS-023 and Spartalizumab (PDR001), MS-023 and Camrelizumab (SHR1210), MS-023 and Sintilimab (IBI308), MS-023 and Tislelizumab (BGB-A317), MS-023 and Toripalimab (JS 001), MS-023 and AMP-224, MS-023 and AMP-514, MS-023 and Atezolizumab (Tecentriq), MS-023 and Avelumab (Bavencio), MS-023 and Durvalumab (Imfinzi), MS-023 and KN035, MS-023 and CK-301, MS-023 and AUNP12, MS-023 and CA-170, MS-023 and BMS-986189, MS-023 and Ipilimumab (Yervoy), MS-023 and Tremelimumab, TC-E 5003 and Pembrolizumab (Keytruda), TC-E 5003 and Nivolumab (Opdivo), TC-E 5003 and Cemiplimab (Libtayo), TC-E 5003 and Spartalizumab (PDR001), TC-E 5003 and Camrelizumab (SHR1210), TC-E 5003 and Sintilimab (IBI308), TC-E 5003 and Tislelizumab (BGB-A317), TC-E 5003 and Toripalimab (JS 001), TC-E 5003 and AMP-224, TC-E 5003 and AMP-514, TC-E 5003 and Atezolizumab (Tecentriq), TC-E 5003 and Avelumab (Bavencio), TC-E 5003 and Durvalumab (Imfinzi), TC-E 5003 and KN035, TC-E 5003 and CK-301, TC-E 5003 and AUNP12, TC-E 5003 and CA-170, TC-E 5003 and BMS-986189, TC-E 5003 and Ipilimumab (Yervoy), TC-E 5003 and Tremelimumab, GSK3368715 and Pembrolizumab (Keytruda), GSK3368715 and Nivolumab (Opdivo), GSK3368715 and Cemiplimab (Libtayo), GSK3368715 and Spartalizumab (PDR001), GSK3368715 and Camrelizumab (SHR1210), GSK3368715 and Sintilimab (IBI308), GSK3368715 and Tislelizumab (BGB-A317), GSK3368715 and Toripalimab (JS 001), GSK3368715 and AMP-224, GSK3368715 and AMP-514, GSK3368715 and Atezolizumab (Tecentriq), GSK3368715 and Avelumab (Bavencio), GSK3368715 and Durvalumab (Imfinzi), GSK3368715 and KN035, GSK3368715 and CK-301, GSK3368715 and AUNP12, GSK3368715 and CA-170, GSK3368715 and BMS-986189, GSK3368715 and Ipilimumab (Yervoy), GSK3368715 and Tremelimumab, GSK3326595 and Pembrolizumab (Keytruda), GSK3326595 and Nivolumab (Opdivo), GSK3326595 and Cemiplimab (Libtayo), GSK3326595 and Spartalizumab (PDR001), GSK3326595 and Camrelizumab (SHR1210), GSK3326595 and Sintilimab (IBI308), GSK3326595 and Tislelizumab (BGB-A317), GSK3326595 and Toripalimab (JS 001), GSK3326595 and AMP-224, GSK3326595 and AMP-514, GSK3326595 and Atezolizumab (Tecentriq), GSK3326595 and Avelumab (Bavencio), GSK3326595 and Durvalumab (Imfinzi), GSK3326595 and KN035, GSK3326595 and CK-301, GSK3326595 and AUNP12, GSK3326595 and CA-170, GSK3326595 and BMS-986189, GSK3326595 and Ipilimumab (Yervoy), GSK3326595 and Tremelimumab, EPZ015666 and Pembrolizumab (Keytruda), EPZ015666 and Nivolumab (Opdivo), EPZ015666 and Cemiplimab (Libtayo), EPZ015666 and Spartalizumab (PDR001), EPZ015666 and Camrelizumab (SHR1210), EPZ015666 and Sintilimab (IBI308), EPZ015666 and Tislelizumab (BGB-A317), EPZ015666 and Toripalimab (JS 001), EPZ015666 and AMP-224, EPZ015666 and AMP-514, EPZ015666 and Atezolizumab (Tecentriq), EPZ015666 and Avelumab (Bavencio), EPZ015666 and Durvalumab (Imfinzi), EPZ015666 and KN035, EPZ015666 and CK-301, EPZ015666 and AUNP12, EPZ015666 and CA-170, EPZ015666 and BMS-986189, EPZ015666 and Ipilimumab (Yervoy), EPZ015666 and Tremelimumab, LLY-283 and Pembrolizumab (Keytruda), LLY-283 and Nivolumab (Opdivo), LLY-283 and Cemiplimab (Libtayo), LLY-283 and Spartalizumab (PDR001), LLY-283 and Camrelizumab (SHR1210), LLY-283 and Sintilimab (IBI308), LLY-283 and Tislelizumab (BGB-A317), LLY-283 and Toripalimab (JS 001), LLY-283 and AMP-224, LLY-283 and AMP-514, LLY-283 and Atezolizumab (Tecentriq), LLY-283 and Avelumab (Bavencio), LLY-283 and Durvalumab (Imfinzi), LLY-283 and KN035, LLY-283 and CK-301, LLY-283 and AUNP12, LLY-283 and CA-170, LLY-283 and BMS-986189, LLY-283 and Ipilimumab (Yervoy), LLY-283 and Tremelimumab, JNJ-64619178 and Pembrolizumab (Keytruda), JNJ-64619178 and Nivolumab (Opdivo), JNJ-64619178 and Cemiplimab (Libtayo), JNJ-64619178 and Spartalizumab (PDR001), JNJ-64619178 and Camrelizumab (SHR1210), JNJ-64619178 and Sintilimab (IBI308), JNJ-64619178 and Tislelizumab (BGB-A317), JNJ-64619178 and Toripalimab (JS 001), JNJ-64619178 and AMP-224, JNJ-64619178 and AMP-514, JNJ-64619178 and Atezolizumab (Tecentriq), JNJ-64619178 and Avelumab (Bavencio), JNJ-64619178 and Durvalumab (Imfinzi), JNJ-64619178 and KN035, JNJ-64619178 and CK-301, JNJ-64619178 and AUNP12, JNJ-64619178 and CA-170, JNJ-64619178 and BMS-986189, JNJ-64619178 and Ipilimumab (Yervoy), JNJ-64619178 and Tremelimumab, PRT543 and Pembrolizumab (Keytruda), PRT543 and Nivolumab (Opdivo), PRT543 and Cemiplimab (Libtayo), PRT543 and Spartalizumab (PDR001), PRT543 and Camrelizumab (SHR1210), PRT543 and Sintilimab (IBI308), PRT543 and Tislelizumab (BGB-A317), PRT543 and Toripalimab (JS 001), PRT543 and AMP-224, PRT543 and AMP-514, PRT543 and Atezolizumab (Tecentriq), PRT543 and Avelumab (Bavencio), PRT543 and Durvalumab (Imfinzi), PRT543 and KN035, PRT543 and CK-301, PRT543 and AUNP12, PRT543 and CA-170, PRT543 and BMS-986189, PRT543 and Ipilimumab (Yervoy), and PRT543 and Tremelimumab.

In some embodiments, the first agent degrades RBM39, e.g., E7820, and the immunotherapeutic agent inhibits PD-1 (e.g., an anti-PD1 antibody or inhibiting ligand; e.g., is selected from is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, and the like).

Any reference herein to particular agents, i.e., the first agent or immunotherapeutic agent (e.g., checkpoint inhibitor), whether in the context of individual or combined application, also encompasses acceptable pro-drugs and acceptable salts thereof as can be determined and understood by persons of ordinary skill in the art.

In some embodiments, the method is performed in vitro, i.e., the cancer cell is maintained in culture where it is contacted with the first agent that modulates RNA splicing, and optionally contacted with the immunotherapeutic agent, as described above.

Notably, the inventors have shown that the exposing target cancer cells in vivo to agents that modulate or perturb RNA splicing sensitizes them to immunotherapeutic agents, thus drastically enhancing the efficacy of such immunotherapies. Accordingly, the disclosure also encompasses methods wherein the cancer cell is contacted with the first agent that modulates RNA splicing in vivo, i.e., in a subject with cancer or suspected of having cancer. The step of contacting the cancer cell comprises administering to the subject a therapeutically effective amount of the agent that modulates RNA splicing, as described above. The method can also comprise administering to the subject a therapeutically effective amount of a checkpoint inhibitor as described herein. Accordingly, in another aspect, the disclosure provides compositions and/or a method for treating a cancer in a subject in need thereof. The method comprises administering to the subject a therapeutically effective amount of a first agent that modulates RNA splicing in cancer cells and a therapeutically effective amount of an immunotherapeutic agent. The treatment can be applied across many cancers and, thus, is not limited to any one cancer. Exemplary cancers applicable to this aspect of the disclosure are described above. Further, exemplary agents serving as the first agent and immunotherapeutic agent, and exemplary combinations thereof, are described in more detail above and are encompassed in this aspect of the disclosure.

The terms “subject” refers to an individual or patient with, or suspected to have, cancer. The subject can be a mammal being assessed for treatment and/or being treated. In certain embodiments, the mammal is a human. While subjects may be human, the term also encompasses other mammals, particularly those mammals useful as laboratory models for human disease, e.g., mouse, rat, guinea pig, rabbit, dog, cat, non-human primate, and the like.

As used herein, the term “treat” refers to medical management of a disease, disorder, or condition (e.g., cancer, as described above) of a subject (e.g., a human or non-human mammal, such as another primate, horse, dog, mouse, rat, guinea pig, rabbit, and the like). Treatment can encompasses any indicia of success in the treatment or amelioration of a disease or condition (e.g., a cancer), including any parameter such as abatement, remission, diminishing of symptoms or making the disease or condition more tolerable to the patient, slowing in the rate of degeneration or decline of the subject, or making the degeneration of the subject less debilitating. Specifically, in the context of cancer, the term treat can encompass slowing or inhibiting the rate of cancer growth, or reducing the likelihood of recurrence, compared to not having the treatment. In some embodiments, the treatment encompasses resulting in some detectable degree of cancer cell death in the patient. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition (e.g., cancer). The term “therapeutic effect” refers to the amelioration, reduction, or elimination of the disease or condition, symptoms of the disease or condition, or side effects of the disease or condition in the subject. The term “therapeutically effective” indicates parameters or qualities that are appropriate to achieve a therapeutic effect. In the context of quantity, the term refers to an amount of the composition that results in a therapeutic effect and can be readily determined. In the context of components or formulations, the term refers to ingredients that facilitate or permit the therapeutic effect without significantly negating the effect or causing significant side-effects.

In some embodiments, the first agent and the immunotherapeutic agent are administered in coordination or combination. In some embodiments, the first agent and the immunotherapeutic agent of the combination are administered within a period of 7 days of each other. Illustrative, non-limiting combinations of first agents and immunotherapeutic agents are described above. For example, a therapeutically effective amount of the first agent can be administered to the subject about 7, 6, 5, 4, 3, 2, or 1 day(s) before a therapeutically effective amount of the immunotherapeutic agent is administered to the subject. Alternatively, a therapeutically effective amount of the immunotherapeutic agent can be administered to the subject about 7, 6, 5, 4, 3, 2, or 1 day(s) before a therapeutically effective amount of the first agent is administered to the subject. In some embodiments, the first agent and the immunotherapeutic agent are administered on the same day. In some embodiments, the first agent and immunotherapeutic agent are administered together, e.g., concurrently, either in the same formulation or separate formulations. As used herein, the term “concurrently” indicates simultaneous administration (e.g., when in the same formulation) or close in time (e.g., within one or a few hours, such as during the same clinic visit). In some embodiments, therapeutically effective amounts of the first agent, and optionally the immunotherapeutic agent, are administered to the subject multiple times after initial diagnosis. In one example, the first agent and the immunotherapeutic agent (e.g., blockade inhibitor) are administered concurrently (i.e., in the same or separate administrations) in multiple doses over time. Such administration regimens can be appropriately designated by the attending physician or care-giver and may also include multiple diagnostic or monitoring assays to determine and inform ongoing dosing regimens. In one illustrative example, E7820 is the first agent that modulates RNA splicing and is administered once daily for a plurality of day (e.g., 2, 3, 4, 5, 6, 7, 8, 9, 10, days, etc.). An exemplary dose of E7820 is about 100 mg/day, based on clinical trial (Phase I) data. This administration can be concurrent with, or otherwise coordinated with, administration(s) of immunotherapeutic agents (e.g., immune checkpoint inhibitors). In some embodiments, the subject has received one or more administrations of the immunotherapeutic agent and it is determined that the efficacy of the immunotherapeutic intervention is not sufficiently effective or is reducing in efficacy. After such determination, the subject receives one or more administrations of the first agent in combination or coordination with continuing administrations of immunotherapeutic agents.

The disclosure also encompasses formulations appropriate for methods of administration for application to in vivo therapeutic settings in subjects (e.g., mammalian subjects with cancer). According to skill and knowledge common in the art, the disclosed first agent that modulates RNA splicing, independent from or optionally in combination with or an immunotherapeutic agent (e.g., immune checkpoint inhibitor), can be formulated with appropriate carriers and non-active binders, and the like, for administration to target specific tumor and/or cancer cells. Illustrative, non-limiting examples of combinations of exemplary first agents that modulates RNA splicing and exemplary immunotherapeutic agents are described above. The disclosure also encompasses formulations that incorporate the first agent (and optionally immunotherapeutic agent, e.g., checkpoint inhibitor agent) in acceptable pro-drug and/or acceptable salt embodiments, as can be determined and understood by persons of ordinary skill in the art.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Current Protocols in Immunology, John Wiley & Sons, New York (2010), Mirzaei, H. and Carrasco, M. (eds.), Modem Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.” Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following experimental description is provided for the purpose of providing those of ordinary skill in the art with a complete disclosure and description of how to make and use the present invention, and is not intended to limit the scope of what the inventors regard as their invention nor is it intended to represent that the experiments below are all or the only experiments performed.

Specifically, the experimental description addresses an exemplary study demonstrating the surprising finding that pharmacologic perturbation of RNA splicing in tumor cells can elicit an anti-tumor immune response and can enhance the anti-tumor activity of immune checkpoint inhibitor therapies.

### Introduction

Neoantigens produced in cancer cells can be determinative of response to immune checkpoint blockade therapy. Although coding DNA mutations are the best-studied source of neoantigens, tumor antigens can arise from other processes as well. While studies have suggested that RNA splicing changes occurring in cancer cells can lead to production of neoantigens, there is no evidence that such neoantigens modulate the endogenous immune response to the cancer cells or sensitize cancer cells to immunotherapeutic agents.

Identifying candidate splicing-derived neoantigens is subject to additional complexities beyond the well-described limitations of in silico predictions of mutation-derived neoantigens. For example, many aberrant splicing events result in production of mRNAs that are retained in the nucleus or degraded in the cytoplasm by nonsense-mediated mRNA decay (NMD), which may either positively or negatively alter their contributions to the MHC I-bound immunopeptidome. These complexities may underlie the difficulty of establishing links between splicing and tumor immunogenicity, exemplified by one report that although intron retention generates neoepitopes, the quantitative extent of intron retention is not associated with response to immune checkpoint blockade.

The question of whether alterations in splicing can generate bona fide, splicing-derived neoantigens is particularly important given the recent identification of multiple clinical-grade compounds that alter RNA splicing catalysis via non-overlapping mechanisms. These include small molecules that inhibit interaction of RNA with the core SF3b splicing complex, such as pladienolide B, GEX1A (also known as herboxidiene), E7107, and H3B-8800 (Kotake, Y., et al. (2007). Splicing factor SF3b as a target of the antitumor natural product pladienolide. Nat Chem Biol 3, 570-575; Lagisetti, C., et al. (2014). Pre-mRNA splicing-modulatory pharmacophores: the total synthesis of herboxidiene, a pladienolide-herboxidiene hybrid analog and related derivatives. ACS Chem Biol 9, 643-648; Lee, S. C., et al. (2016). Modulation of splicing catalysis for therapeutic targeting of leukemia with mutations in genes encoding spliceosomal proteins. Nat Med 22, 672-678; Seiler, M., et al. (2018). H3B-8800, an orally available small-molecule splicing modulator, induces lethality in spliceosome-mutant cancers. Nat Med 24, 497-504; Sellin, M., et al. (2019). The Splicing Modulator GEX1A Exhibits Potent Anti-Leukemic Activity Both in Vitro and In Vivo through Inducing an MCL1 Splice-Switch in Pre-Clinical Models of Acute Myeloid Leukemia. Blood 134, 2666-2666; and Yokoi, A., et al. (2011). Biological validation that SF3b is a target of the antitumor macrolide pladienolide. FEBS J 278, 4870-4880; each of which is incorporated herein by reference in its entirety). More recently, a series of compounds known as “anti-cancer sulfonamides,” including indisulam and E7820, were found to perturb RNA splicing by inducing ubiquitination and proteasomal degradation of the accessory splicing factor RBM39. These drugs, which have been studied in phase I/II clinical trials for both hematologic and solid cancers, have a mechanism of action highly analogous to the FDA-approved drug lenalidomide (Jan, M., et al. (2021). Cancer therapies based on targeted protein degradation—lessons learned with lenalidomide. Nat Rev Clin Oncol, 1-17; Kronke, J., et al. (2015). Lenalidomide induces ubiquitination and degradation of CKlalpha in del(5q) MDS. Nature 523, 183-188; Kronke, J., et al. (2014). Lenalidomide causes selective degradation of IKZF1 and IKZF3 in multiple myeloma cells. Science 343, 301-305; each of which is incorporated herein by reference in its entirety), as they render RBM39 a novel substrate for the Ddb1/CUL4 E3 ubiquitin ligase complex (Han, T., et al. (2017). Anticancer sulfonamides target splicing by inducing RBM39 degradation via recruitment to DCAF15. Science, 356(6336); Uehara, T., et al. (2017). Selective degradation of splicing factor CAPERalpha by anticancer sulfonamides. Nat Chem Biol 13, 675-680; Wang, R. F., et al. (1996). Utilization of an alternative open reading frame of a normal gene in generating a novel human cancer antigen. J Exp Med 183, 1131-1140; each of which is incorporated herein by reference in its entirety). Treatment with either RBM39 degraders or SF3b inhibitors results in alterations in splicing which generate novel, unannotated mRNA sequences in a dose-dependent manner. Additionally, blocking post-translational modifications of splicing factors, which are required for spliceosome assembly and effective splicing catalysis, can robustly perturb splicing. For example, RNA splicing factors are the most heavily arginine-methylated proteins in cells. As such, drugs which block either asymmetric or symmetric arginine dimethylation by inhibiting type I or type II protein arginine methyltransferase enzymes (PRMTs) can potently perturb RNA splicing Fedoriw, A., et al. (2019). Anti-tumor Activity of the Type I PRMT Inhibitor, GSK3368715, Synergizes with PRMT5 Inhibition through MTAP Loss. Cancer Cell 36, 100-114 e125; Fong, J. Y., et al. (2019). Therapeutic Targeting of RNA Splicing Catalysis through Inhibition of Protein Arginine Methylation. Cancer Cell 36, 194-209 e199; Koh, C. M., et al. (2015). MYC regulates the core pre-mRNA splicing machinery as an essential step in lymphomagenesis. Nature 523, 96-100; Radzisheuskaya, A., et al. (2019). PRMT5 methylome profiling uncovers a direct link to splicing regulation in acute myeloid leukemia. Nat Struct Mol Biol 26, 999-1012; each of which is incorporated herein by reference in its entirety).

Here, the central question of whether altered RNA splicing generates immunologically meaningful neoantigens to provoke an effective anti-tumor immune response is addressed. In parallel, a therapeutic approach is identified to boost tumor antigen production with specific classes of splicing modulatory compounds. The study reveals that these compounds are tolerated by the immune system and induce splicing-derived, MHC I-bound antigens that trigger an endogenous T cell response, and demonstrates that these drugs can be efficaciously combined with anti-PD1 to enhance response to checkpoint blockade. As several of the studied compounds have proven safe in early phase clinical trials, these studies provide a practical means to enhance immunotherapies in a clinical setting.

### Results

Pharmacologic Perturbation of Splicing Suppresses Tumor Growth In Vivo in a Manner Dependent on Host T Cells and Tumoral MHC I-Presented Peptides

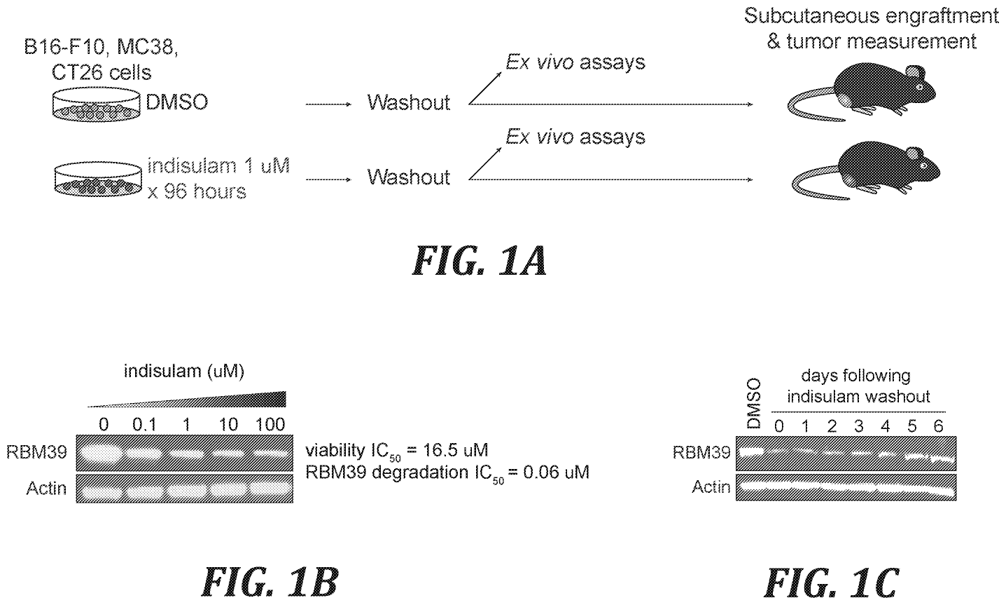

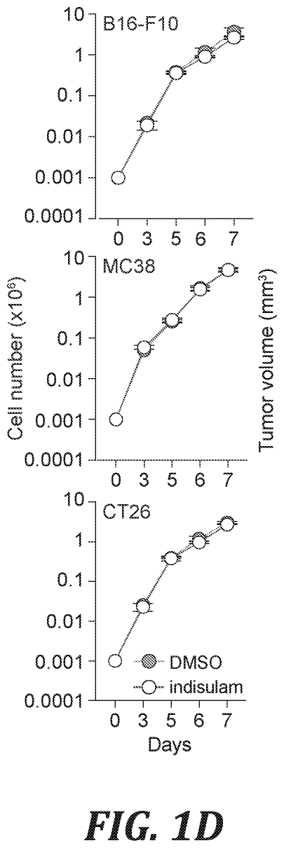

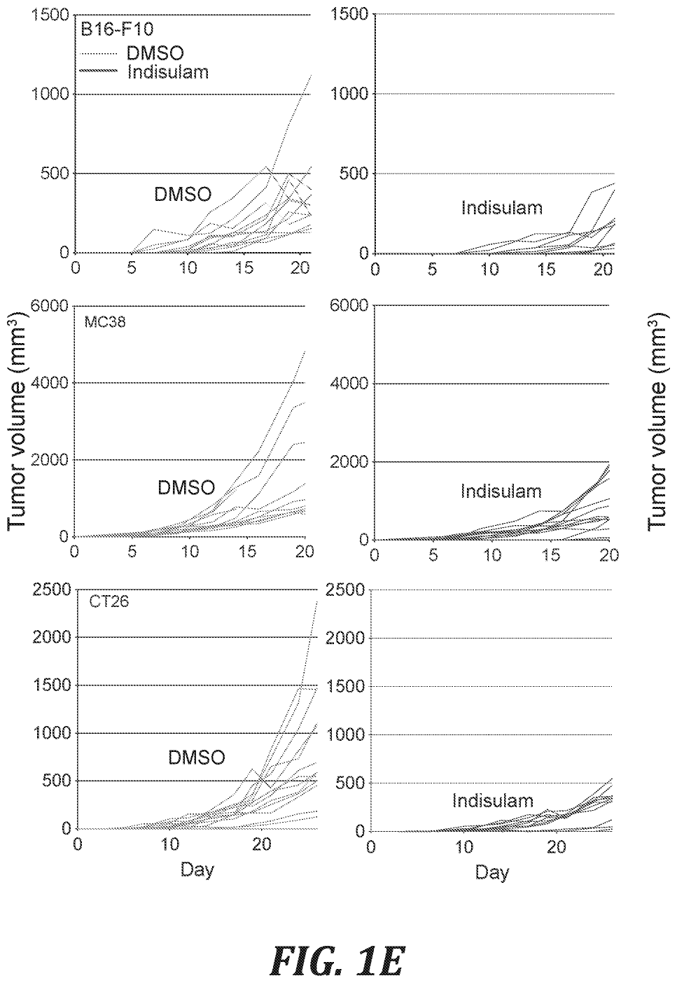

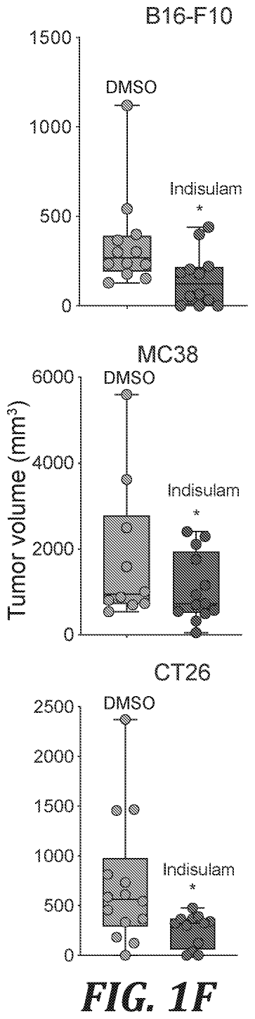

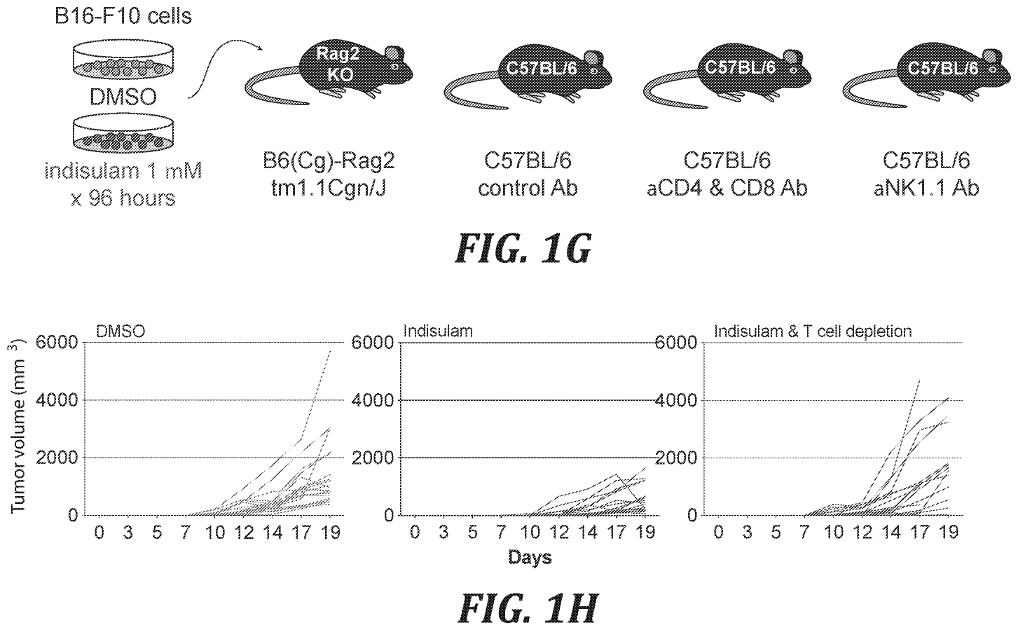

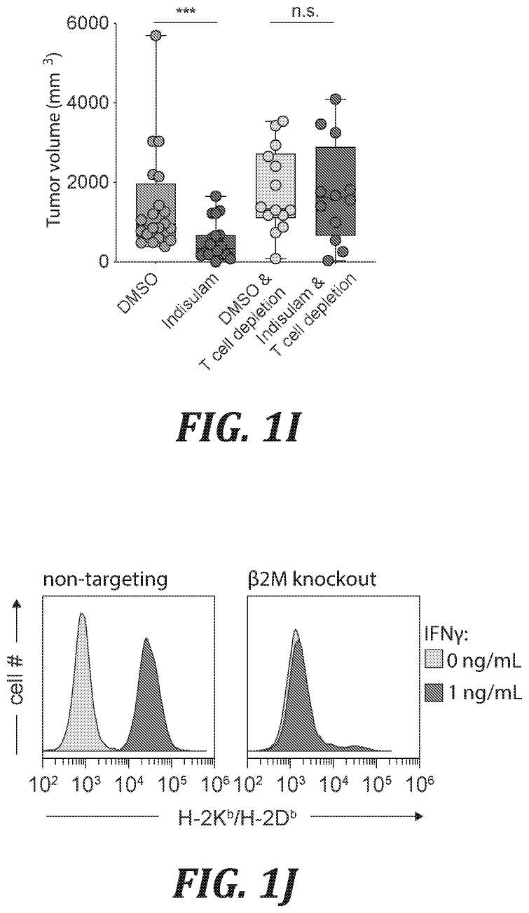

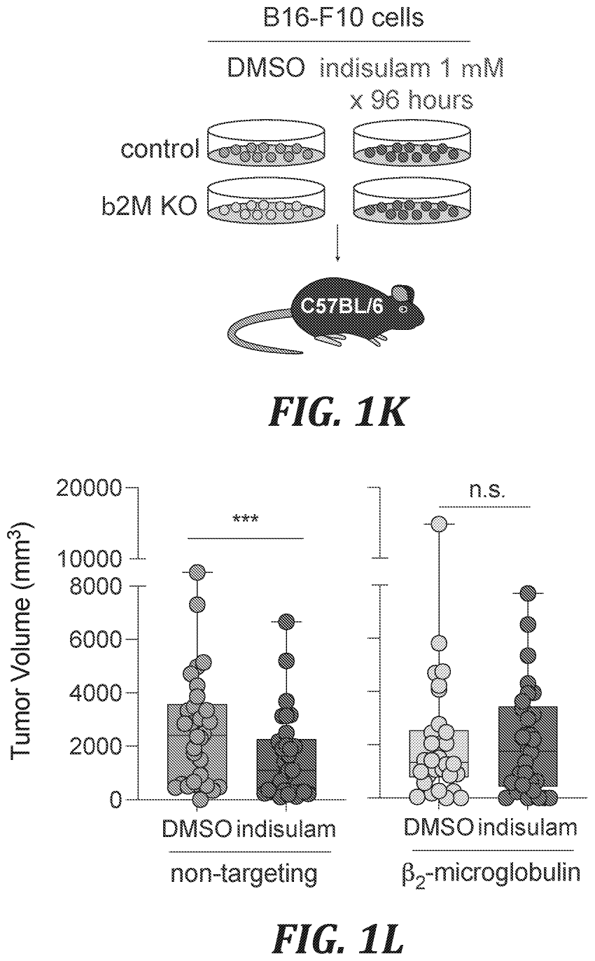

It was hypothesized that pharmacologically perturbing splicing might generate aberrant mRNA transcripts encoding novel proteins, a subset of which could be translated, processed, and presented by MHC class I as neoepitopes that increase tumor antigenicity and provoke immune cell rejection. First, this hypothesis was tested by treating a variety of mouse cancer cell lines (B16-F10 melanoma, MC38 colon cancer, and CT26 colon cancer cells) with the RBM39-degrading compound indisulam at doses that are subinhibitory for growth (FIG. 1A). Indisulam treatment resulted in dose-dependent degradation of RBM39 but few effects on cell growth in vitro, with IC₅₀ values for RBM39 degradation ranging from 0.06-1.0 μM and IC₅₀ values for growth ranging from 2.1-73 μM (FIG. 1B for results in B16-10 cells; similar results in MC38, CT26, and LLC cells not shown). Across these cell lines, ex vivo treatment with indisulam at 1 μM for four days followed by drug washout yielded sustained suppression of RBM39 protein for days following drug removal but had minimal effects on subsequent cell proliferation or apoptosis (FIGS. 1C-1D). Moreover, drug treatment did not change cell-surface expression of MHC I, MHC II, PD-L1, or cytokine or death receptors such as IFNγ receptors, TNF receptors, or DR5.

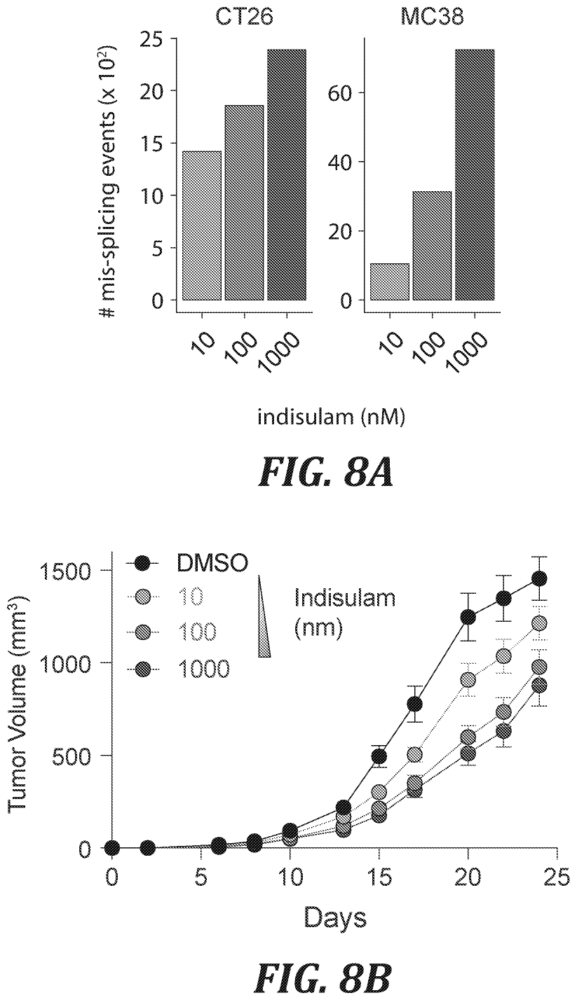

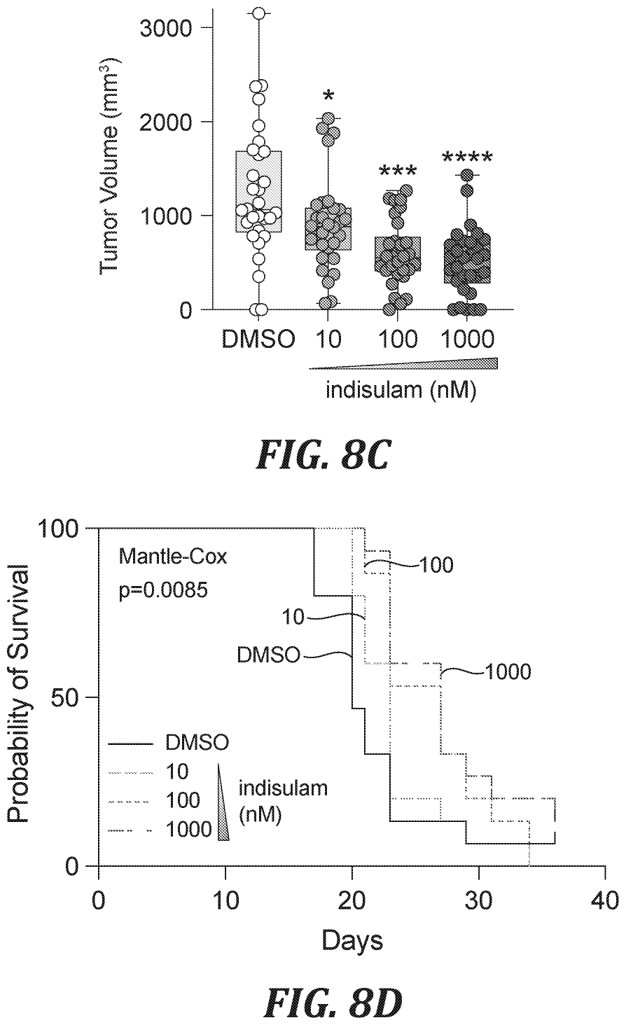

In contrast to the minimal effects on cell growth in vitro, identically treated cells exhibited strikingly durable growth inhibition following engraftment into syngeneic, immunocompetent mice in vivo, despite only transient prior drug exposure (FIGS. 1E-1F). This growth inhibition following engraftment was dose dependent, with exposure to increasing drug concentrations resulting in increased splicing alterations (FIG. 8A), reduced tumor growth in vivo (FIGS. 8B and 8C), and improved animal survival (FIG. 8D).

The discrepancy between in vitro versus in vivo growth after transient exposure to splicing modulatory compounds suggested non-tumor cell autonomous effects. To assess the possibility that RBM39 degradation, and consequent splicing derangements, might be stimulating an anti-cancer immune response, the above experiments were repeated but treated B16-F10 cells were engrafted into immunocompromised Rag2-deficient C57BL/6 mice and, separately, into wild-type C57BL/6J mice with or without T cell depletion (using anti-CD4 and anti-CD8 antibodies) or natural killer (NK) cell depletion (using anti-NKT.1 antibody; FIG. 1G). These experiments revealed that the in vivo tumor growth inhibition from transient indisulam treatment was rescued in B and T cell-deficient Rag2 recipients as well as by T cell depletion, suggesting a T cell-dependent mechanism for growth inhibition by indisulam (FIGS. 1H-1I). By contrast, NK depletion did not rescue cell growth (not shown). These observations led to a hypothesis that T cells were the dominant immune cell type important for response to perturbed splicing in tumor cells and hinted at antigen-dependent immune effects.

To evaluate whether antigen presentation on MHC I and recognition by CD8⁺ T cells in particular could be responsible for the effect observed, the effects of indisulam versus DMSO pretreatment of isogenic B16-F10 cells with or without cell-surface MHC I via CRISPR-mediated knockout of B2m, encoding β₂-microglobulin were evaluated next (FIGS. 1J-1K). Loss of β₂-microglobulin rescued the growth inhibition induced by indisulam pretreatment of wild-type control B16-F10 (FIG. 1L). Overall, these results indicate that RBM39 degradation impaired cancer cell growth in a manner dependent on T cells and MHC I expression in tumor cells.

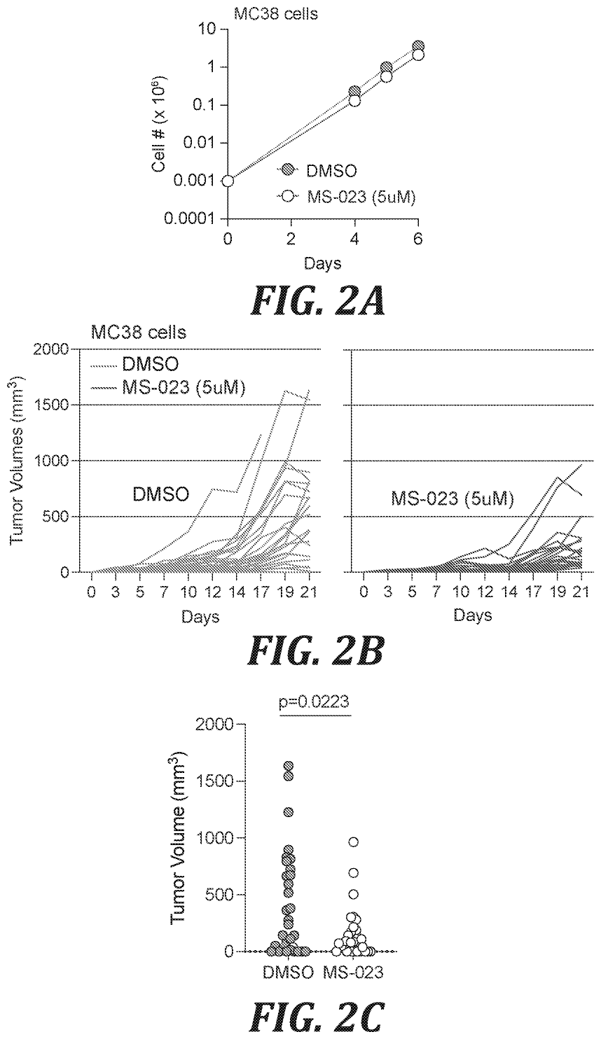

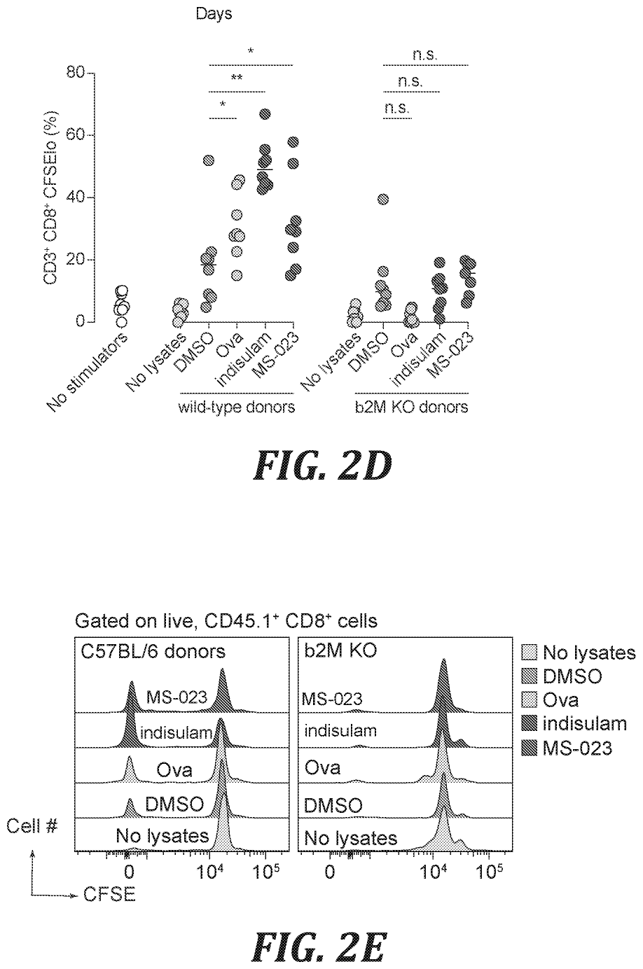

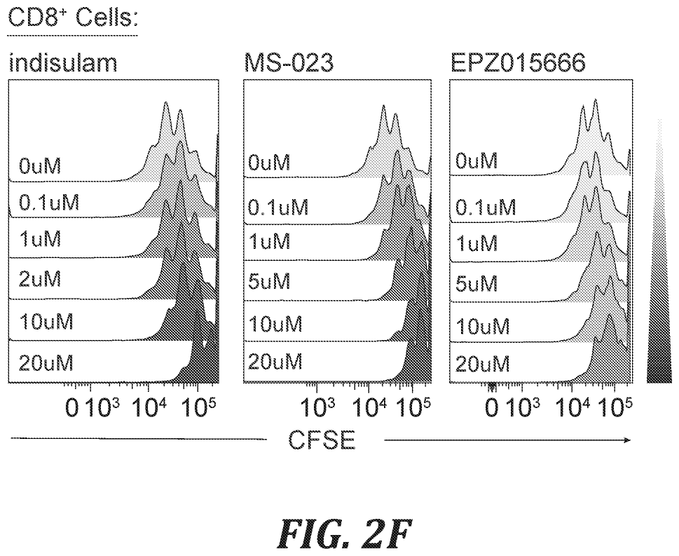

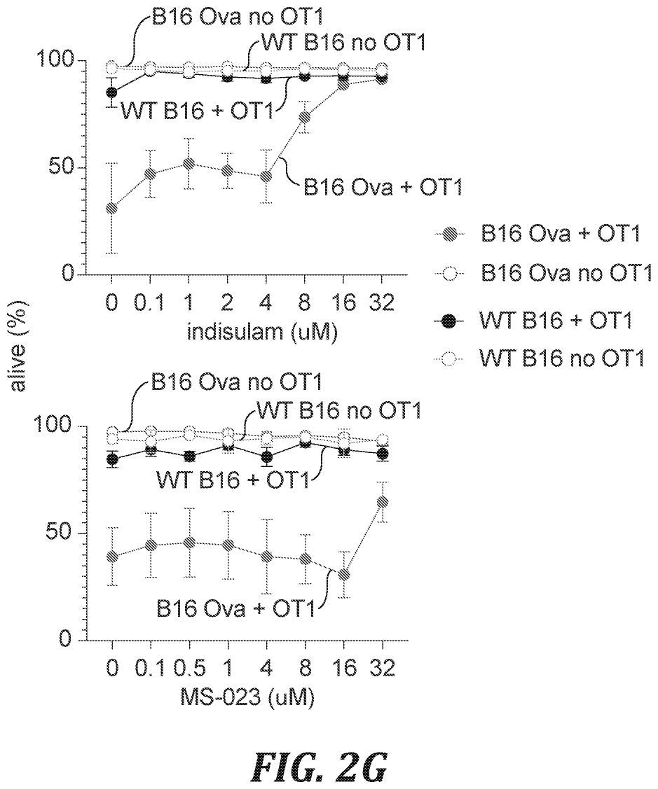

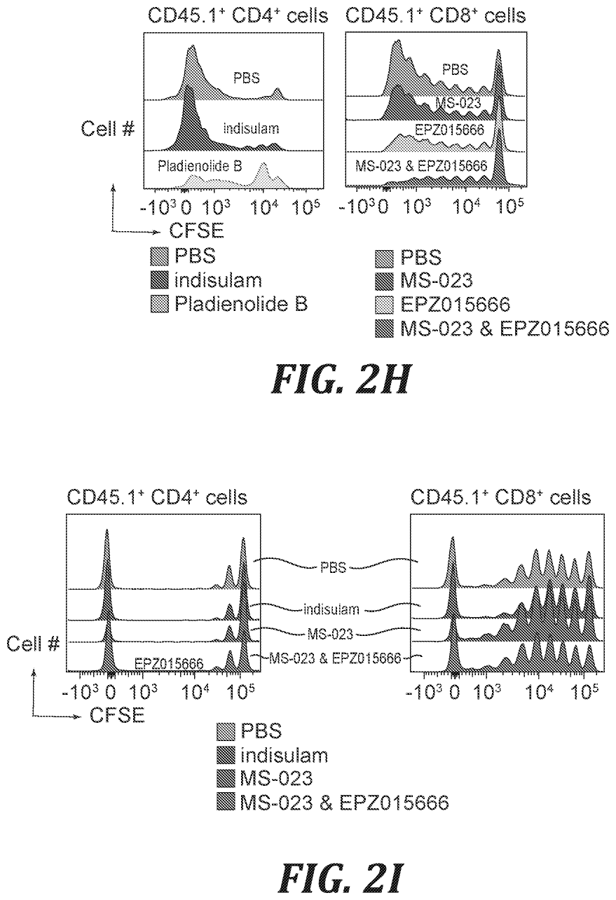

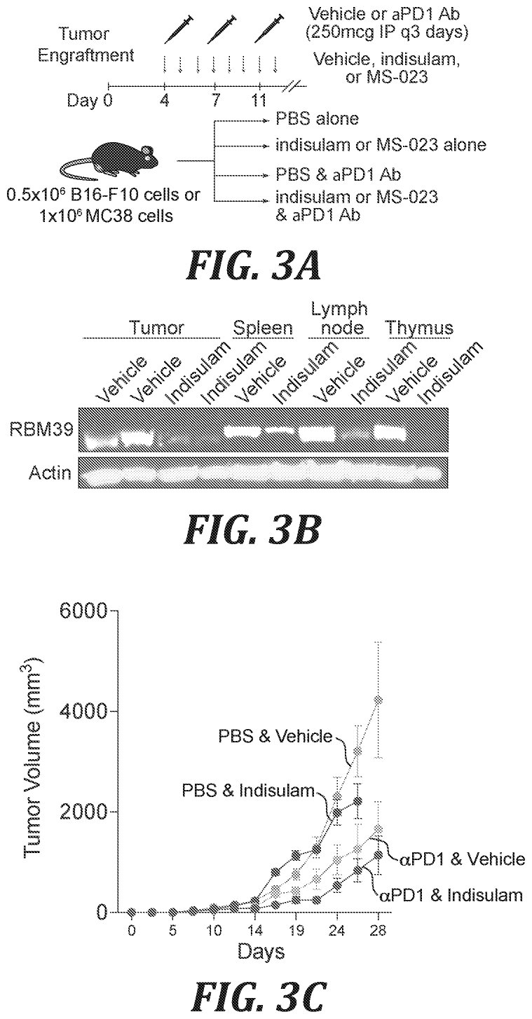

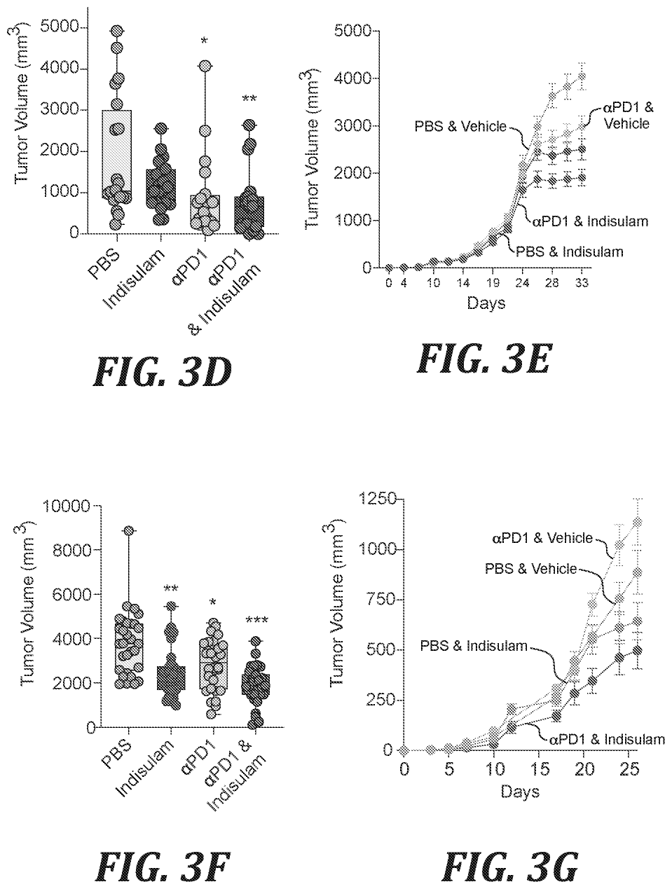

Importantly, the apparently immune-mediated effects of splicing modulation on tumor growth were also observed for the mechanistically distinct drug MS-023, which modulates splicing by inhibiting Type I PRMT enzymes (Eram, M. S., et al. (2016). A Potent, Selective, and Cell-Active Inhibitor of Human Type I Protein Arginine Methyltransferases. ACS Chem Biol 11, 772-781; incorporated herein by reference in its entirety). Ex vivo treatment of MC38 cells with concentrations of MS-023 that are subinhibitory for in vitro growth (5 μM for 96 hours) resulted in globally reduced asymmetric dimethyl arginines (ADMA), with minimal effects on cell growth in vitro after drug washout (FIG. 2A). Much as for RBM39 degradation, however, strikingly different behavior was observed in vivo. These same MS-023-treated cells exhibited durable suppression of tumor growth following engraftment into syngeneic, immune-competent mice in vivo (FIGS. 2B-2C). These data suggest that the effects of pharmacologic splicing modulation on tumor growth in vivo can be generalized to multiple tumor types and splicing modulatory drugs that act via distinct mechanisms.

Next, it was tested whether pharmacologic modulation of splicing enhances tumor immune recognition via drug-induced neoantigen production, rather than through other possible mechanisms, and whether professional antigen-presenting cells (APCs) are involved in enhanced recognition of drug-treated tumors. To explore this, the ability of antigen-presenting cells loaded with lysates derived from control versus drug-treated tumor cells to stimulate naïve, syngeneic T cells in traditional mixed leukocyte reactions was compared. Briefly, in these experiments, MC38 cells were treated with DMSO, indisulam or MS-023, and used to generate lysates containing potentially immunogenic peptides, but no active drug or viable tumor cells. Bone marrow-derived dendritic cells (BMDCs) from wild-type C57BL/6 or B2m knockout mice were pulsed with these lysates, washed and used in a mixed leukocyte reaction with CFSE-labeled, naïve, syngeneic CD45.1⁺CD5⁺ splenic T cells. BMDCs loaded with peptide-containing lysates from cells treated with indisulam or MS-023 more strongly promoted CD8⁺ T cell proliferation than did DMSO- or no lysate-pulsed control BMDCs (FIGS. 2D-2E). Of note, a CFSE^{lo} percentage of ˜50%, assuming 6 cell divisions, corresponds to an initial reactive population of 50/(2⁶) or 0.7%, providing an estimate of the possible frequency of naïve T cells that are reactive to potential splicing-associated neoantigens. Notably, this effect was not observed when B2m knockout BMDCs were used, indicating that presentation of peptides on MHC I was critical for the observed phenomena. These data are consistent with the hypothesis that in vivo growth suppression of splicing modulator-treated cells arises in part from reactive T cell action against neoantigens presented by MHC I. This point is particularly relevant for Type I PRMT inhibition, as inhibition of ADMA may have multiple cellular effects beyond splicing perturbation (in contrast to indisulam, whose effects can be attributed almost entirely to on-target degradation of RBM39).

Effect of Splicing Inhibition on T Cell Activation, Proliferation, and Function

The initial studies were performed in the controlled setting of ex vivo treatment of cancer cells with splicing modulatory drugs, permitting evaluation of the effects of inducing splicing dysregulation in the tumor cell alone. As such, they did not assess effects of in vivo treatment using splicing modulators, which would affect immune and hematopoietic compartments in addition to the tumor itself. To address this, the effects of a variety of drugs which perturb RNA splicing on T cell function were first systematically evaluated. These compounds included indisulam, MS-023, the Type II PRMT (PRMT5) inhibitor EPZ015666 (Chan-Penebre, E., et al. (2015). A selective inhibitor of PRMT5 with in vivo and in vitro potency in MCL models. Nat Chem Biol 11, 432-437, incorporated herein by reference in its entirety), and the SF3b inhibitors pladienolide B (Kotake, Y., et al. (2007). Splicing factor SF3b as a target of the antitumor natural product pladienolide. Nat Chem Biol 3, 570-575; and Yokoi, A., et al. (2011). Biological validation that SF3b is a target of the antitumor macrolide pladienolide. FEBS J 278, 4870-4880, each of which is incorporated herein by reference in its entirety) and GEX1A (Gamboa Lopez, A., et al. (2021). Herboxidiene Features That Mediate Conformation-Dependent SF3B1 Interactions to Inhibit Splicing. ACS Chem Biol 16, 520-528; Ghosh, A. K., et al. (2021). Design and synthesis of herboxidiene derivatives that potently inhibit in vitro splicing. Org Biomol Chem 19, 1365-1377; and Lagisetti, C., et al. (2014). Pre-mRNA splicing-modulatory pharmacophores: the total synthesis of herboxidiene, a pladienolide-herboxidiene hybrid analog and related derivatives. ACS Chem Biol 9, 643-648, each of which is incorporated herein by reference in its entirety), both of which broadly inhibit splicing by disrupting interactions between SF3B1 and pre-mRNA.

The next step was to test how each compound affected T cell activation and proliferation in vitro and in vivo. First, the effects of increasing doses of each drug on the proliferation of CFSE-labeled, purified CD5⁺ splenic T cells following anti-CD3 and CD8 antibody stimulation were evaluated. Despite three days of continuous drug exposure, indisulam and the PRMT inhibitors had minimal effects on T cell proliferation following stimulation (IC₅₀ values of ˜1-10 μM) compared to the SF3b inhibitors pladienolide B and GEX1A, which were markedly inhibitory (IC₅₀ values in the low nanomolar range) of T cell proliferation (FIG. 2F). Measurement of T cell apoptosis and activation markers confirmed that the tested SF3b inhibitors in particular were profoundly immunosuppressive, while indisulam and MS-023 exerted much milder effects (not shown).

The effects of indisulam and MS-023 on T cell function in detail were assessed next. Because the studied SF3b inhibitors suppressed T cell activation and proliferation and induced apoptosis, these were not included in subsequent assays. Indisulam and MS-023 each minimally impaired the in vitro cytotoxicity of primed OT-1 transgenic T cells against ovalbumin (OVA)-expressing B16-F10 cells (FIG. 2G), with minimal impairment of tumor cell killing by T cells at doses less than 4 μM. Similar results were observed for MC38 cells (not shown). Additionally, even exposure to higher doses of indisulam or MS-023 did not inhibit the ability of OT-1 T cells to secrete IFNγ or TNFα (not shown), or their ability to degranulate intracellular cytolytic molecules (not shown).

These functional studies were complemented by determining how each compound affected the gene expression program of activated T cells. T cells were stimulated with anti-CD3 and CD28 antibodies ex vivo in the presence of DMSO, indisulam, MS-023, EPZ015666, or pladienolide B. Genes that were normally upregulated upon T cell activation were markedly attenuated by pladienolide B, and to a lesser extent by EPZ015666, whereas indisulam or MS-023 caused much milder changes (not shown), consistent with the effects of each compound on T cell proliferation following stimulation.

Finally, the effect of each drug on in vivo T cell activation and proliferation in response to alloantigen was evaluated. In these assays, CFSE-labeled purified CD5⁺ splenic T cells from CD45.1 congenic mice were adoptively transferred into lethally irradiated recipients which were either syngeneic (wild-type C57BL/6), mismatched for non-MHC “minor” antigens (LP/J), or major MHC mismatched (Balb/c; H-2^{b} vs. H-2^{d}) as previously described (Lu, S. X., et al. (2008). STAT-3 and ERK 1/2 phosphorylation are critical for T-cell alloactivation and graft-versus-host disease. Blood 112, 5254-5258, incorporated herein by reference in its entirety). Recipient animals were treated daily with each splicing modulatory compound from one day prior to adoptive transfer until euthanasia. Adoptive T cell transfer was followed by daily in vivo administration of vehicle, indisulam, MS-023, EPZ015666, or combined Type I PRMT and PRMT5 inhibition at doses used in prior anti-tumor studies that result in target engagement in vivo to assess the effects of these compounds on T cell activation and proliferation. In this system, transfer of C57BL/6 CD45.1 T cells into Balb/c recipients resulted in robust CD8⁺ and CD4⁺ T cell activation and proliferation in response to alloantigen, as expected (FIG. 2H). Interestingly, while in vivo treatment with indisulam, MS-023, or EPZ015666 resulted in minimal impairment of T cell proliferation or activation (not shown), combined inhibition of both Type I and II PRMTs blocked T cell proliferation (FIGS. 2H-2I). The SF3b inhibitor pladienolide B similarly markedly suppressed T cell proliferation in vivo (FIG. 2H), as expected from our in vitro studies.

In the C57BL/6→Balb/c MHC-mismatched model, nearly all donor T cells are very strongly activated due to mismatches between T cell receptors (TCR) against the recipient H-2 molecules themselves, rather than the peptides presented within. Although the effects of splicing modulatory drugs on T cell activation and proliferation were most prominent in this MHC-mismatched model, they were also recapitulated in the more physiologic B6→LP/J model. In this model, where both strains share the H-2^{b} haplotype, donor T cells recognize “minor” antigens on self-MHC I molecules (FIG. 2I), a scenario more akin to the presentation of neoantigenic peptides in the context of self-MHC. These splicing compounds were additionally permissive to the homeostatic proliferation of T cells in syngeneic adoptive transfer experiments (CD45.1→C57BL/6; not shown).

Lastly, the effects of splicing modulators on hematopoiesis were assessed in methylcellulose assays of bone marrow hematopoietic stem and progenitor cells. These assays demonstrated that normal hematopoiesis was intact at even high (supratherapeutic) micromolar doses of MS-023 and indisulam, whereas EPZ015666 suppressed hematopoiesis at similar doses (not shown). The SF3b inhibitor pladienolide B even more profoundly suppressed hematopoiesis at nanomolar concentrations (not shown). These data indicate that certain classes of drugs which perturb splicing are strongly immunosuppressive or myelosuppressive, while others have negligible effects on T cell activation, proliferation, and function, as well as hematopoiesis, at doses that are therapeutic in preclinical models.

Modulating Splicing Boosts Response to Immune Checkpoint Blockade

Based on the findings above that RBM39 degradation or Type I PRMT inhibition in tumor cells prompts growth suppression without impairing T cell function, it was hypothesized that perturbing RNA splicing might promote control of small, established tumors in the context of immune checkpoint blockade. Thus, the effects of in vivo RBM39 degradation alone or in combination with anti-PD1 were evaluated. In these experiments, C57BL/6 mice were engrafted with syngeneic cancer cell lines (B16-F10, MC38, or LLC cells), followed by in vivo treatment with vehicle, indisulam, anti-PD1, or combined indisulam and anti-PD1. Treatment with splicing inhibitors started on day 3 and anti-PD1 therapy on day 7, which is the approximate date range at which tumors became established and measurable. Simultaneous RBM39 degradation and anti-PD1 therapy led to significantly reduced growth of both B16-F10 and MC38 tumors in vivo that exceeded the effects of either treatment alone, indicating at least an additive effect of these therapies (FIGS. 3A-3F). This therapeutic effect occurred at indisulam doses which resulted in on-target RBM39 protein reduction in tumoral as well as immune tissues in vivo (FIG. 3B). Importantly, similar benefits were observed in LLC tumors, which are well-known to be resistant to anti-PD1 therapy. After LLC engraftment into syngeneic C57BL/6 animals, treatment with anti-PD1 therapy alone did not confer a benefit, as expected. However, indisulam monotherapy inhibited tumor growth, which was further accentuated by combining indisulam with anti-PD1 (FIGS. 3G-3H). These data demonstrate an ability of splicing modulation to sensitize immune checkpoint blockade-resistant tumors to immune recognition.

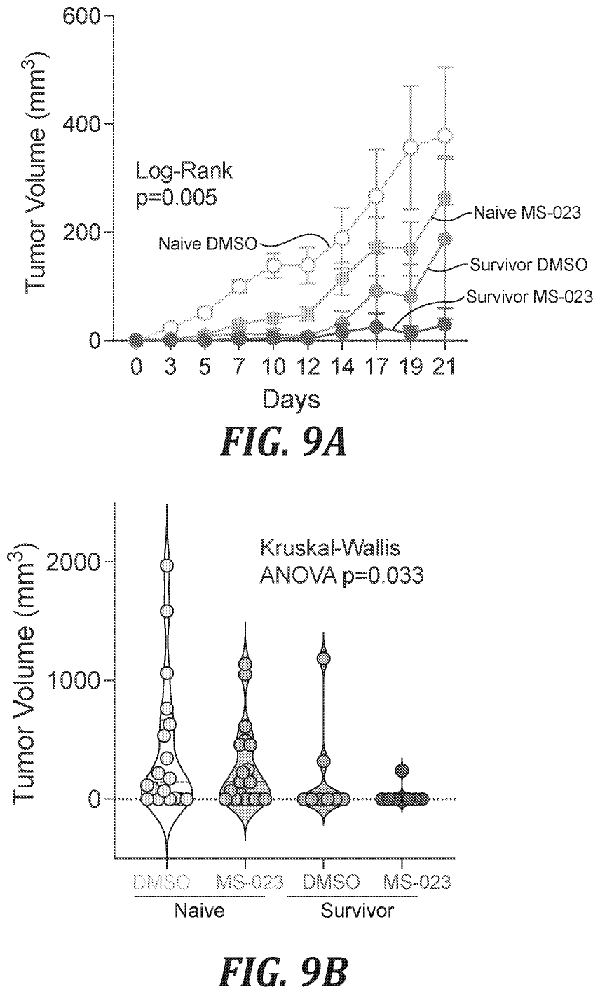

To evaluate whether other means of splicing inhibition could also augment the response to anti-PD1, the impact of combined MS-023 and anti-PD1 treatment was next evaluated in vivo in the same B16-F10 and MC38 models. As with indisulam, in vivo MS-023 treatment significantly improved the response to anti-PD1 therapy (FIGS. 31-3L). Moreover, in mice implanted with MC38 cells, combined MS-023 and anti-PD1 resulted in 50% of mice being alive and tumor-free three months following tumor implantation. In contrast, only 25% of mice treated with either MS-023 or anti-PD1 alone were alive and tumor-free at this time point (p<0.001; FIG. 3M). Notably, surviving animals treated with combined MS-023 and anti-PD1 demonstrated immune memory. When mice that completely rejected MC38 tumors following treatment with MS-023 and anti-PD1 were re-challenged 6 months later with MC38 tumors (with or without MS-023 pretreatment in vitro before engraftment), they exhibited markedly improved tumor control (FIGS. 9A and 9B). In contrast, naïve age-matched, unmanipulated C57BL/6J mice exhibited normal tumor growth as expected.

Finally, undesired pathologies were assessed in non-tumor tissues following exposure to splicing inhibitors with or without anti-PD1 treatment in animals. Extended treatment with either indisulam or MS-023 for three weeks with or without anti-PD1 had negligible effects on lymphocytes, neutrophils, or other peripheral blood counts (not shown). Evaluation of the immune cell content in MC38 tumors following treatment revealed a statistically significant increase in the proportion of CD8⁺ cells amongst hematopoietic cells within tumors when indisulam or MS-023 were delivered in conjunction with anti-PD1, consistent with intra-tumoral T cell expansion mediating the phenotype (not shown). Moreover, treatment of tumor-bearing mice with indisulam, MS-023, anti-PD1, or the combination did not result in overt histologic inflammation or increased immune infiltrates in the skin, lung, gut, or liver (not shown), all common sites of immune-related adverse events observed with clinical anti-PD1 therapy. Concordantly, RNA-seq analyses of lung and colonic tissue as well as splenic T cells purified from indisulam-treated animals showed only mild changes in splicing (not shown), and pathway analyses of differentially regulated genes in these tissues did not reveal an inflammatory signature (not shown).

Splicing Modulators Drive Widespread Production of RNA Isoforms Encoding Predicted Neoepitopes

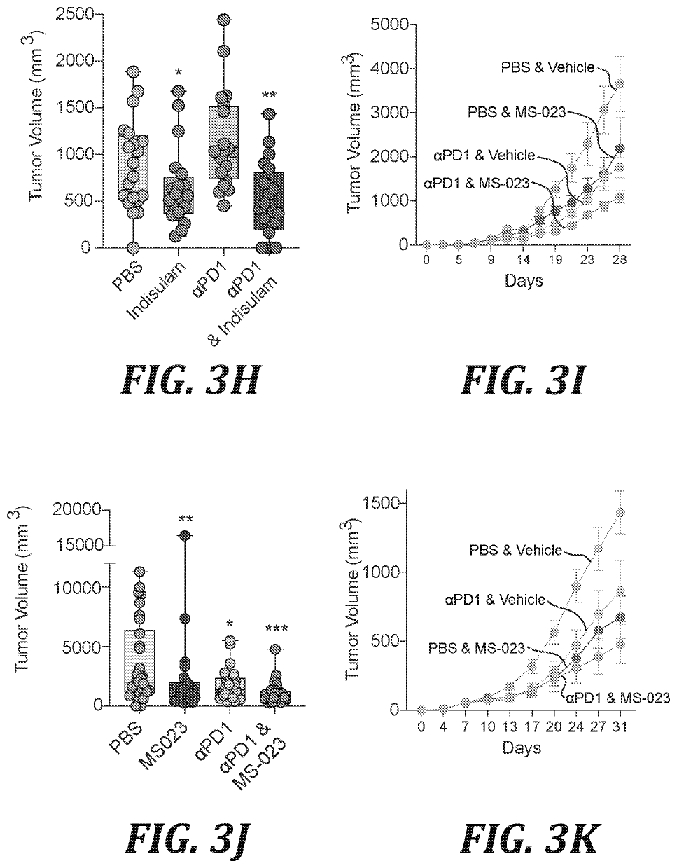

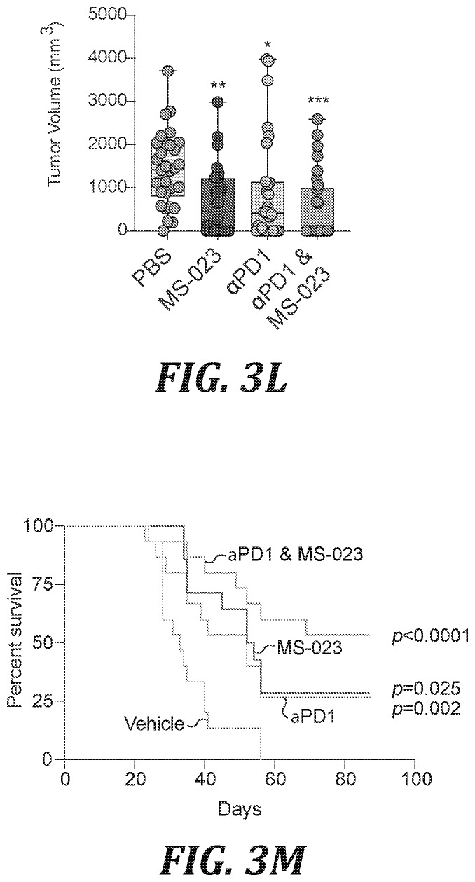

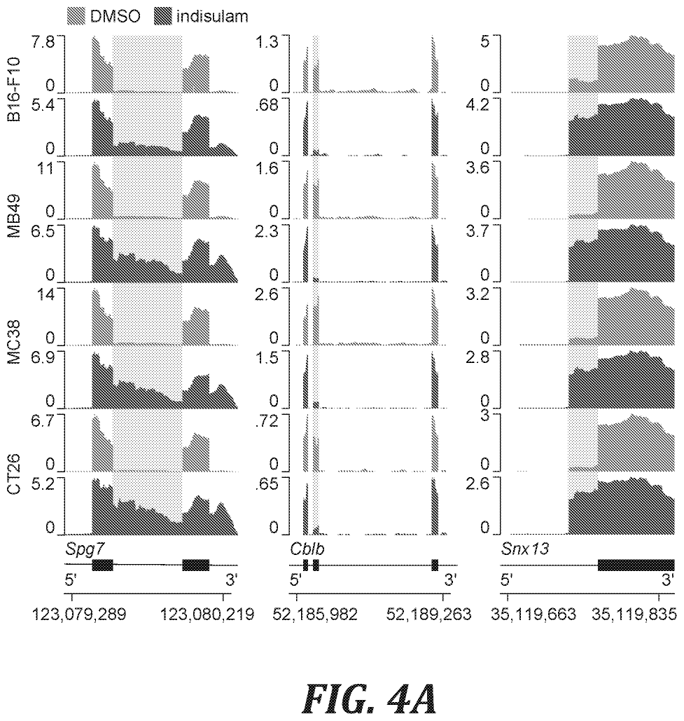

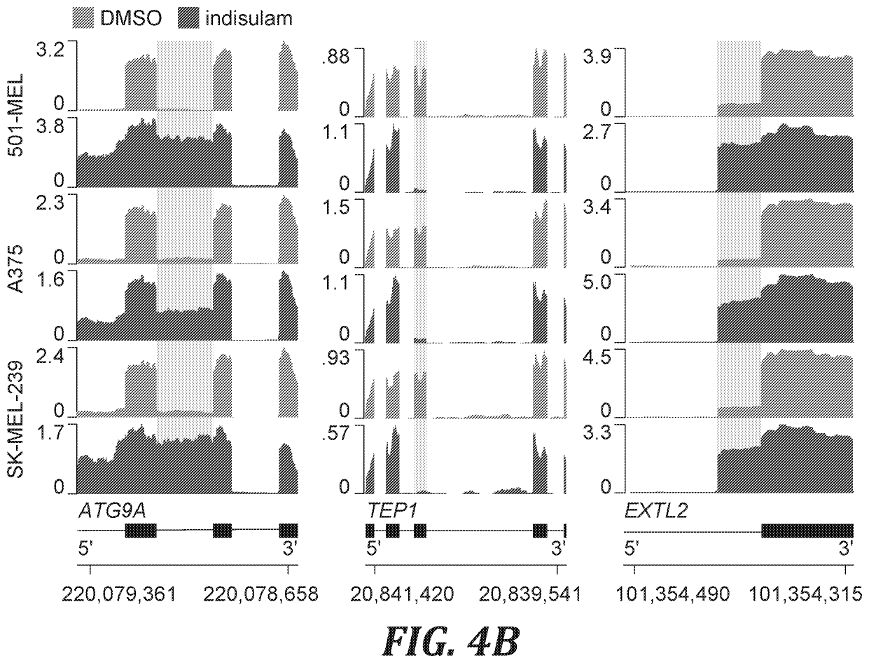

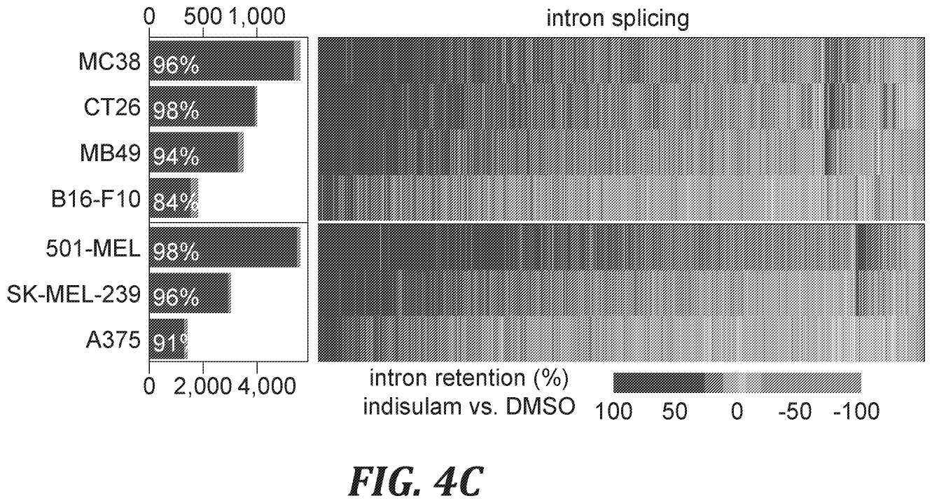

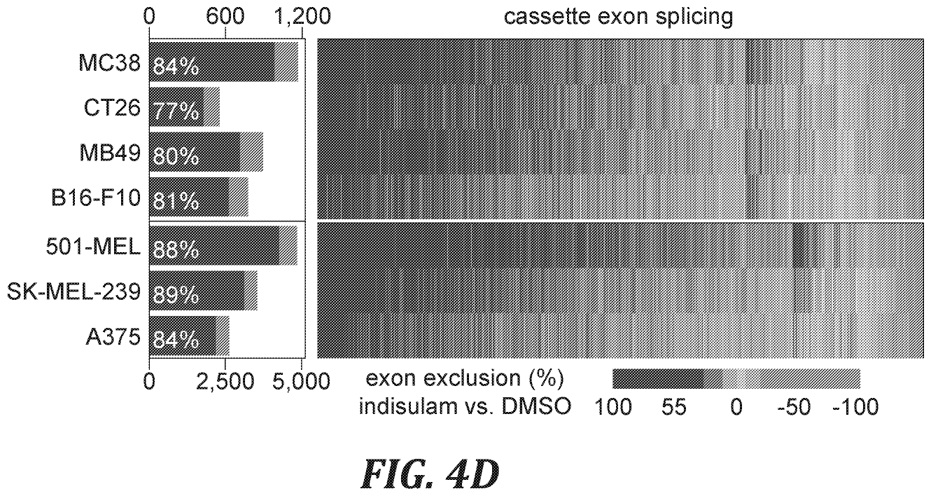

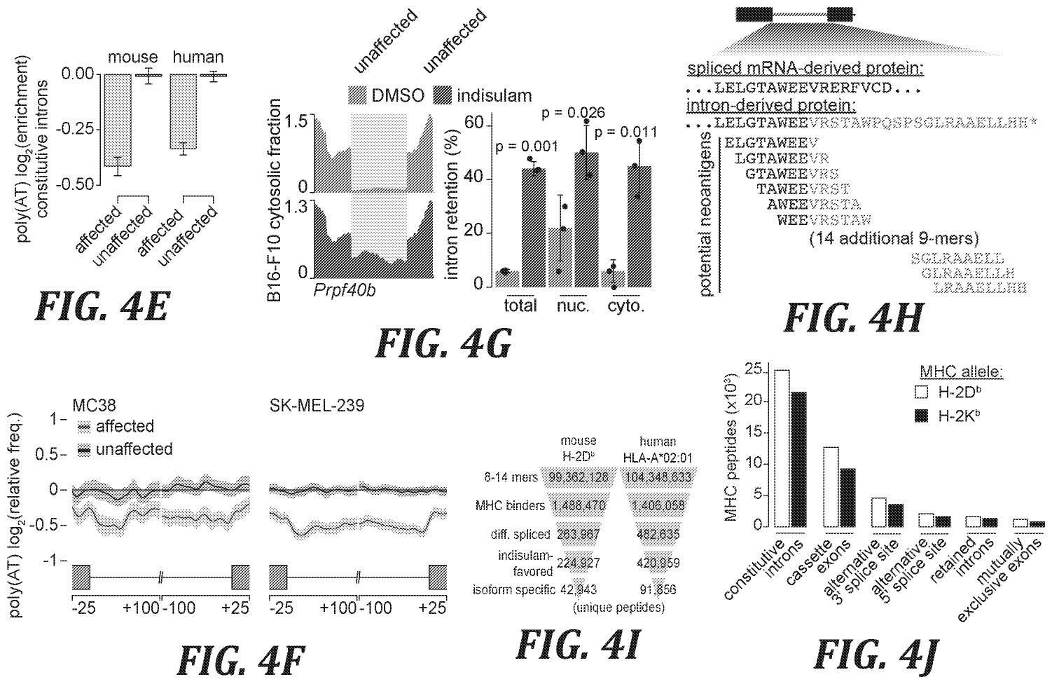

The next step was to determine the molecular mechanisms by which splicing modulation enhances immune-mediated tumor clearance. First, it was determined how splicing modulation altered tumor cell transcriptomes. Four mouse tumor cell lines (B16-F10, MB49, MC38, and CT26) were treated with DMSO, indisulam, or MS-023 at doses that did not affect tumor cell growth in vitro, high-coverage RNA-seq were performed in biological triplicate, and differential gene and isoform expression was quantified in each tumor model. Treatment with either indisulam or MS-023 drove dramatic changes in both alternative and constitutive splicing, affecting cassette exons, competing 5′ and 3′ splice sites, and retained as well as normally constitutive introns (not shown). Differential cassette exon inclusion and constitutive intron splicing were the most common alterations caused by both drugs (FIG. 4A). Next, identical experiments were performed in three human tumor cell lines (501-MEL, A375, and SK-MEL-239). Both indisulam and MS-023 drove stereotyped and pervasive splicing changes in human cancer cells as well, with preferential effects on cassette exons and constitutive introns (FIG. 4B). A subset of mis-splicing events were consistently induced across all tested cancer cell lines in a given species (not shown). Additionally, 29.0% (indisulam) and 9.1% (MS-023) of genes that were mis-spliced in either species were mis-spliced in both (not shown), consistent with the conservation of splicing mechanisms between species.

Although indisulam and MS-023 both drove widespread splicing alterations, they gave rise to distinct downstream splicing alterations, consistent with their different mechanisms of action (not shown). In both mouse and human cancer cells, indisulam-induced splicing alterations were dominated by reduced splicing efficiency: cassette exons were preferentially not included and constitutive introns were preferentially not excised (FIGS. 4C-4D). MS-023 treatment, in contrast, resulted in more balanced splicing changes, with cassette exons and constitutive introns exhibiting both increased and decreased recognition (not shown). Despite the different mechanisms of action of each drug, convergent mis-splicing between indisulam and MS-023 was relatively common in both mouse (4.1-8.3% of mis-spliced events) and human (4.4-8.7% of mis-spliced events) cells (not shown). Constitutive introns that were preferentially retained following indisulam treatment were significantly depleted for poly(AT) sequences, while unaffected constitutive introns exhibited no such signal (FIGS. 4E-4F). As RBM39 preferentially binds poly(AT) motifs, these data are consistent with on-target degradation of RBM39 driving the observed splicing changes. For MS-023, in contrast, no such obvious motif enrichment was observed for affected cassette exons or constitutive introns, consistent with the broad effects of Type I PRMT inhibition on multiple spliceosomal proteins rather than a single, sequence-specific factor like RBM39.

The potential cytoplasmic availability of mis-spliced mRNAs for translation was evaluated next. Because effective splicing is linked to nuclear export of mRNA to the cytoplasm, drug-induced splicing alterations could potentially fail to yield novel peptides. Nuclear and cytoplasmic RNA pools were separated from DMSO- and indisulam-treated cells (focusing on indisulam, given that it led to global decreases in splicing efficiency), sequenced to high coverage, confirmed that the fraction was highly specific (not shown), and drug-induced isoforms in each subcellular compartment were quantified. These experiments revealed that indisulam-induced intron retention is readily apparent in both nuclear and cytoplasmic fractions. For example, levels of an unspliced intron in Prpf40b were ˜3-fold higher in the nuclear versus cytoplasmic fractions of DMSO-treated cells, as expected; however, retention of this intron was highly similar across subcellular compartments following indisulam treatment (FIG. 4G). Similarly marked intron retention across the cytoplasmic transcriptome was observed (not shown), where intron-containing mRNAs would be available for translation into potential neoepitopes (FIG. 4H).

In view of the above results, the potential consequences of indisulam-induced splicing alterations for neoepitope production were estimated. For each studied cell line, all 8-14 amino acid sequences (8-14-mers) arising from mRNA isoforms in the corresponding human or mouse transcriptome were enumerated and the binding affinity of each epitope to common MHC I alleles with NetMHCPan 4.0 was estimated. This list was first restricted to predicted binders, then restricted to epitopes arising from genes that were significantly differentially spliced following indisulam treatment, and finally restricted to epitopes that arose from mRNA isoforms that were promoted by indisulam treatment in that cell line. This filtering dramatically reduced the space of potentially relevant epitopes—for example, from ˜100,000,000 to ˜43,000 in B16-F10 cells (mouse H-2D^{b}, H-2K^{b}; not shown) and ˜92,000 in 501-MEL cells (human HLA-A*02:01; not shown)—with the bulk of such epitopes arising from cassette exons and constitutive introns (FIGS. 4I-4J). Substantial fractions of predicted mis-splicing-derived neoantigens were shared across all tested cell lines in both mouse (5,764) and human (24,378) cells (not shown). In contrast, fewer predicted neoantigens (1,763) were shared between mouse and human cells (not shown), presumably reflecting both non-conserved splicing alterations as well as differences in the binding preferences for murine H-2 molecules versus human HLA.

Drug-Induced, Splicing-Derived Neoepitopes are Presented by MHC I on Tumor Cells

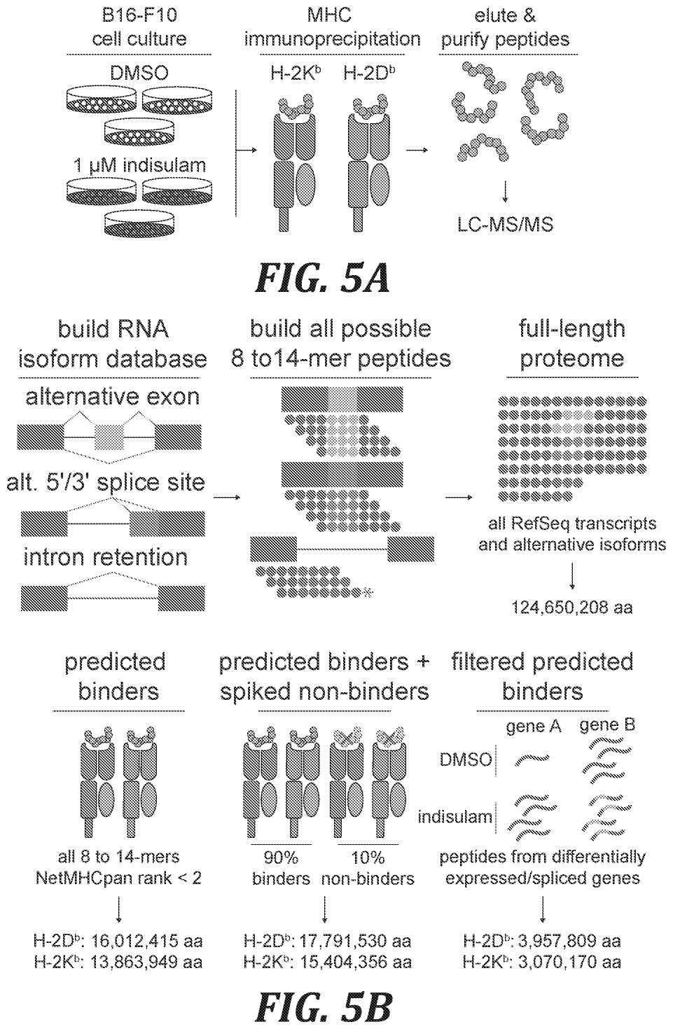

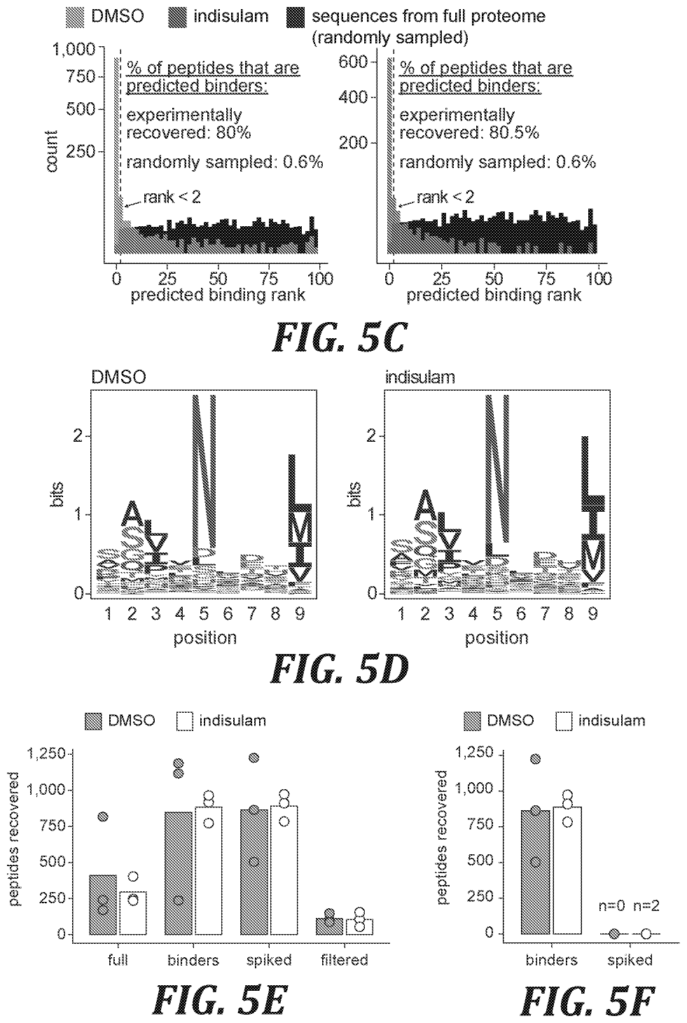

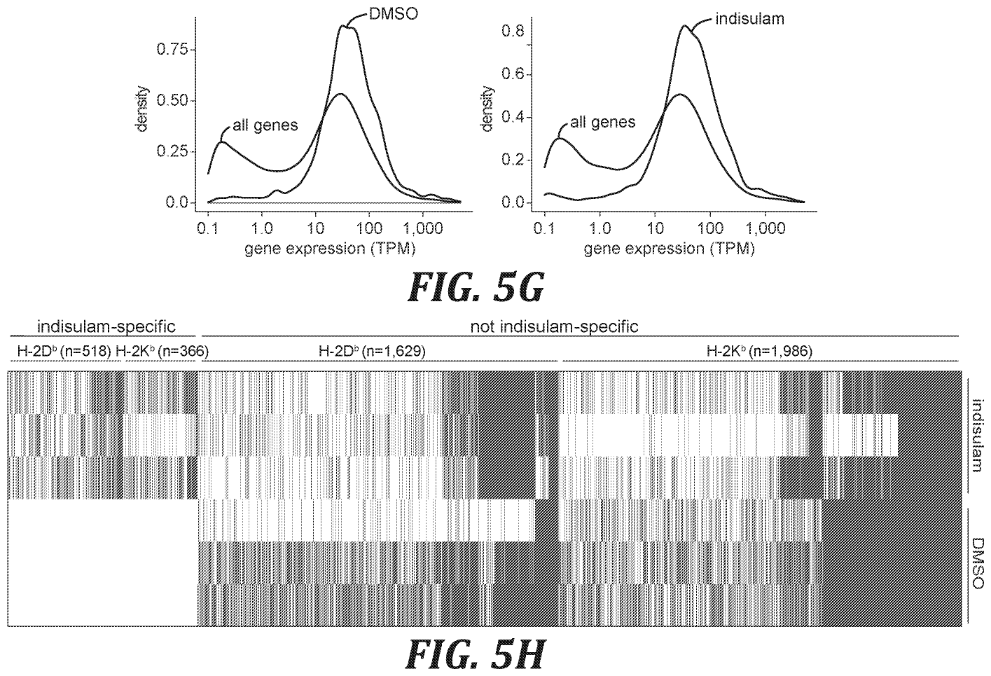

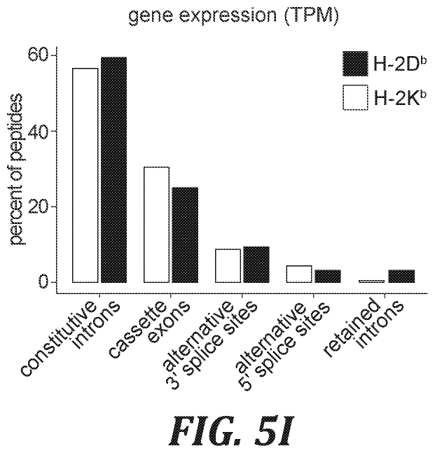

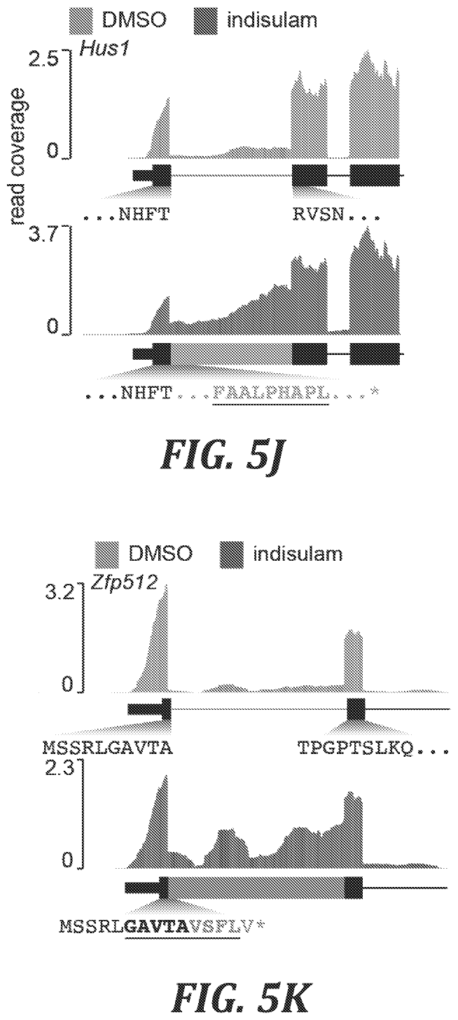

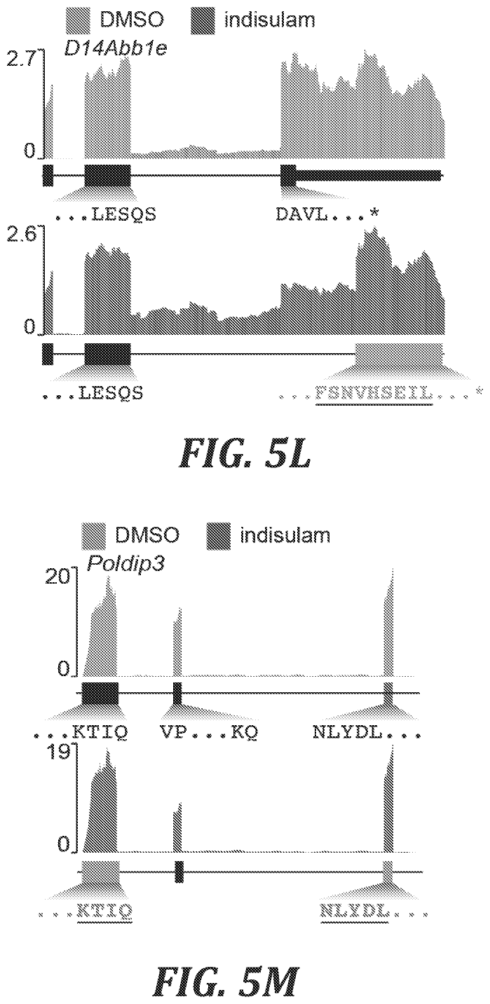

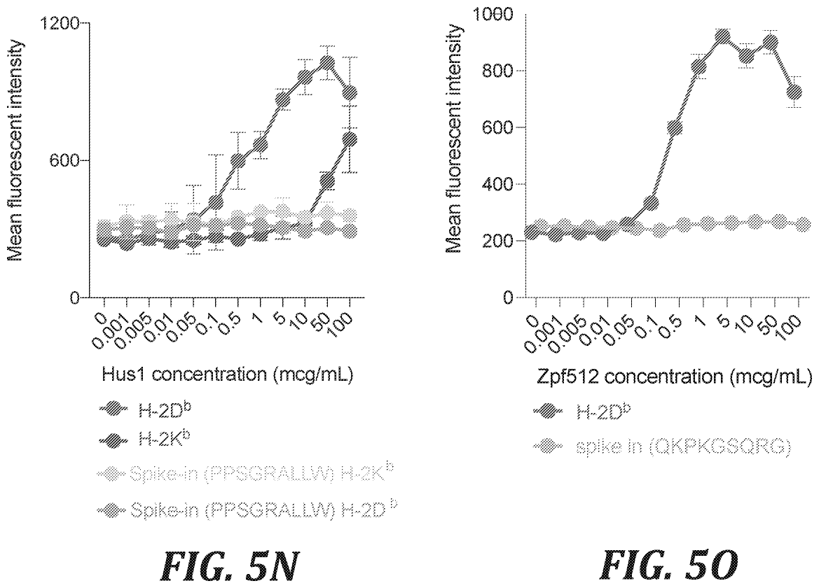

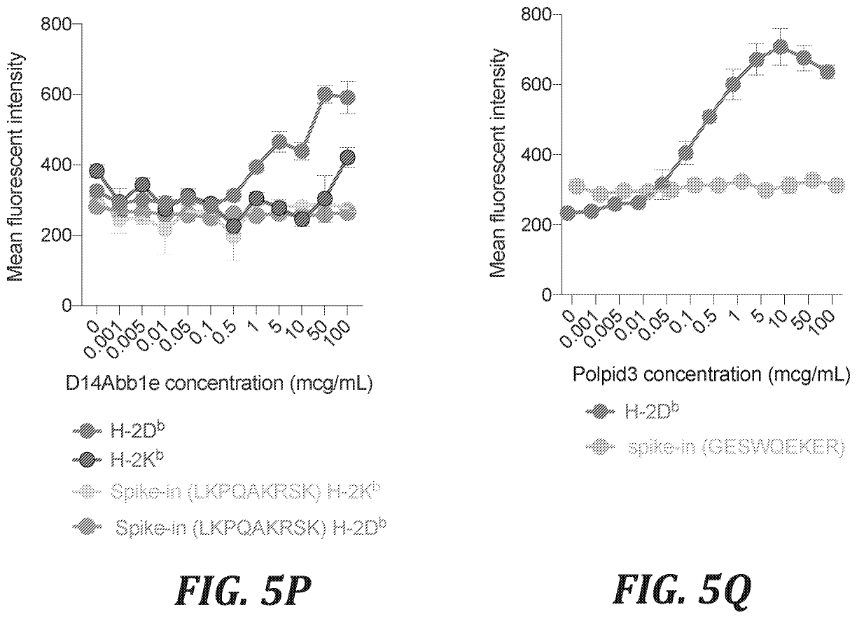

The disclosed transcriptome analyses illustrated the potential for splicing modulation to drive neoepitope production, but did not prove this occurs given the known limitations of in silico prediction of MHC I-bound peptides. Accordingly, the next step was to experimentally identify splicing-derived neoepitopes. B16-F10 cells exposed to 10 U/mL mouse IFNγ to upregulate MHC I were cultured with DMSO or indisulam; H-2Kb and H-2db were separately purified; bound peptides were eluted; and liquid chromatography-tandem mass spectrometry (LC-MS/MS) was performed, all in biological triplicate (FIG. 5A).

Because MHC I-bound peptide identification from mass spectrometry depends critically upon the search database (proteome), four distinct proteomes for each MHC allele were built and tested. These were “full-length proteome,” consisting of all full-length protein sequences in the transcriptome; “predicted binders,” restricted to 8-14-mers that were predicted MHC I binders; “predicted binders+spiked non-binders”, augmented with 8-14-mers that were predicted non-binders as decoys; and “filtered predicted binders,” restricted to predicted binders from differentially expressed or spliced genes (FIG. 5B). MHC I allele binding affinity with NetMHCpan 4.0 was predicted, defining binders as peptides with percentile rank<2 and non-binders as peptides with rank>90 (following the algorithm's recommend threshold for binding).

First, the fidelity of the assay was evaluated by identifying MHC I-bound epitopes with the full-length proteome. Approximately 80% and 86% of identified peptides were predicted binders for H2-D^{b} and H-2K^{b} immunoprecipitations from both DMSO- and indisulam-treated cells, versus 0.6% and 0.9% for peptides that were randomly sampled from the proteome (see FIG. 5C for H2-D^{b} immunoprecipitation; similar results for H-2K^{b} immunoprecipitation were observed (not shown)). Repeating this analysis with MHCflurry-based binding predictions yielded similar results (data not shown). Identified peptides exhibited the expected sequence preferences at anchor residues for both H-2D^{b} and H-2K^{b} for both treatments (see FIG. 5D for H2-D^{b} immunoprecipitation; similar results for H-2K^{b} immunoprecipitation were observed (not shown)), as well as preferential identification of 9-mers and 8-9-mers for H-2D^{b} and H-2K^{b}, respectively, for both treatments (not shown). These analyses suggested that the purification successfully recovered true MHC I-bound epitopes.

Next, the input proteome was varied in order to maximize peptide identification. Restricting the search space to predicted binders increased recovery˜2-fold relative to the full-length proteome, while further restricting to the smaller set of differentially expressed or spliced genes decreased recovery ˜3.4-fold, for the H-2D^{b} immunoprecipitation (FIG. 5E). Similar differences were observed in recovery of ˜1.7-fold and 3.3-fold, respectively, for the H-2K^{b} immunoprecipitation (not shown). Restricting to predicted binders did not decrease specificity: only 2 predicted non-binders were identified, versus 2,204 predicted binders, across all six replicates when the spiked non-binder proteome were queried for the H-2D^{b} immunoprecipitation (FIG. 5F). The H-2K^{b} immunoprecipitation was similarly specific, with 1 predicted non-binder identified versus 2,312 predicted binders across all six replicates (not shown). As the predicted binders proteome maximized yield while minimizing false positives, it was used for subsequent analyses. The vast majority of peptides identified with this proteome from both H-2D^{b} and H-2K^{b} immunoprecipitations arose from genes that were expressed at moderate to high levels in B16-F10 cells treated with DMSO or indisulam (see FIG. 5G for H2-D^{b} immunoprecipitation; similar results for H-2K^{b} immunoprecipitation were observed (not shown)), providing biological support of the analysis's specificity.

Although the majority of peptides were shared (unchanged) between DMSO- and indisulam-treated cells, a substantial subset were treatment-specific. 518 and 366 peptides were identified for H-2D^{b} and H-2K^{b} that were only recovered from indisulam-treated samples (FIG. 5H). Those peptide sets were intersected with predicted isoform-specific epitopes identified by RNA-seq for each allele (FIG. 4J) to obtain 42 and 28 peptides that were bound by H-2D^{b} and H-2K^{b}, respectively, and arose from mRNA isoforms that were specifically promoted by indisulam treatment (FIG. 5I).

Due to the known limited sensitivity of mass spectrometry for analyzing the MHC I immunopeptidome, an additional 39 candidate peptides that were supported by the RNA-seq data alone, but nonetheless predicted to be high-affinity binders to H-2D^{b} or H-2K^{b}, were also selected for further study. This set of 109 (70 from mass spectrometry, 39 from RNA-seq predictions) high-confidence, potentially antigenic peptides was used as input for subsequent functional assays.

Splicing-Derived Neoepitopes are Neoantigens that Trigger an Endogenous T Cell Response

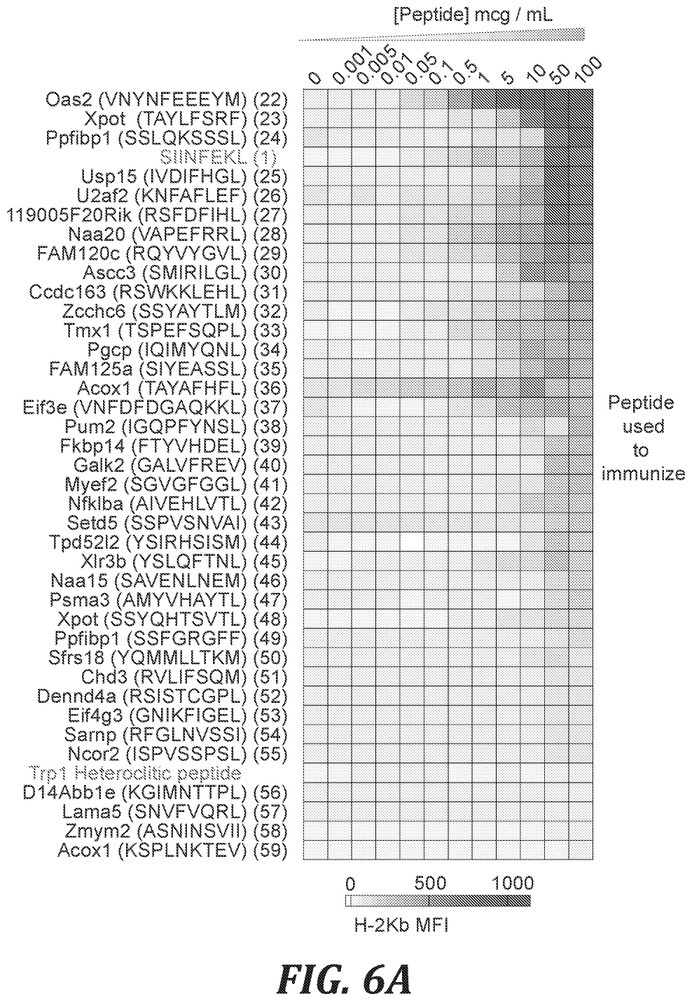

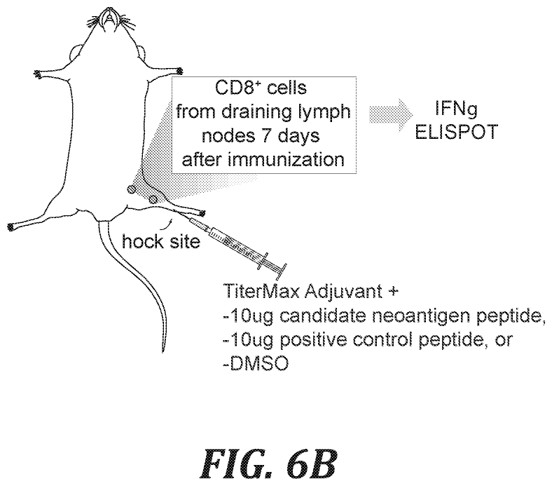

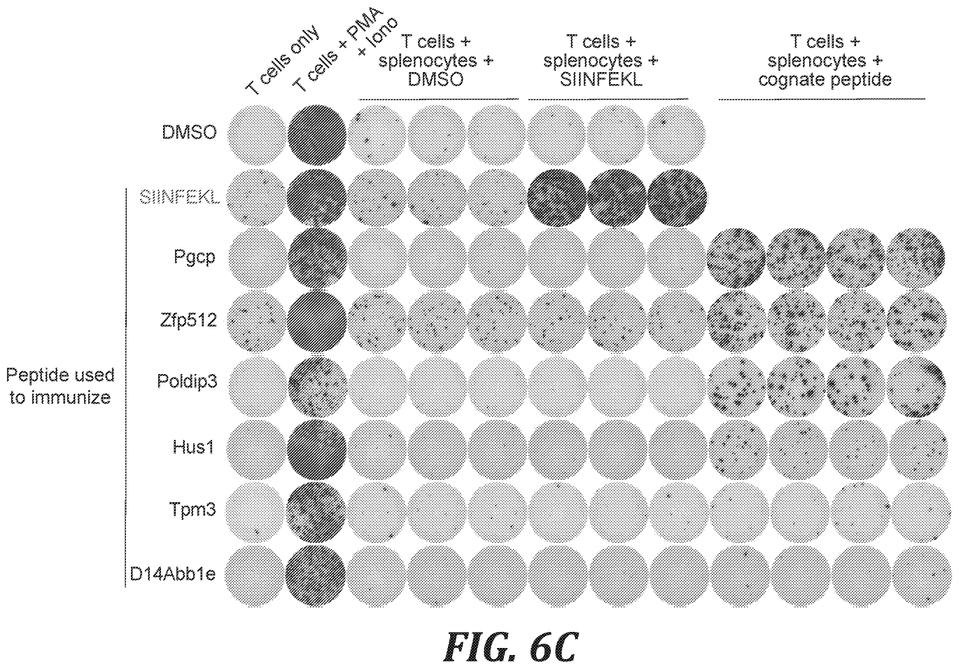

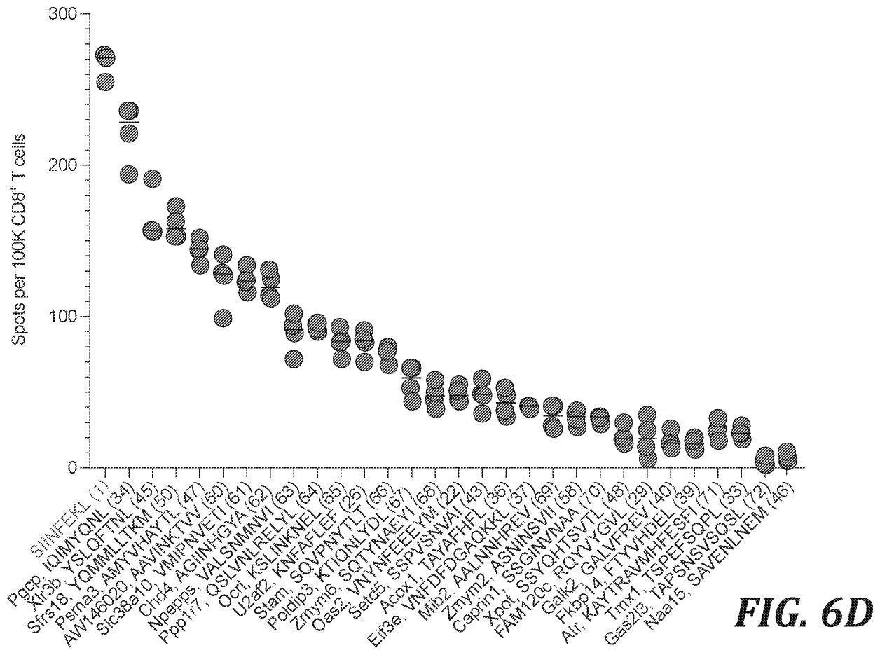

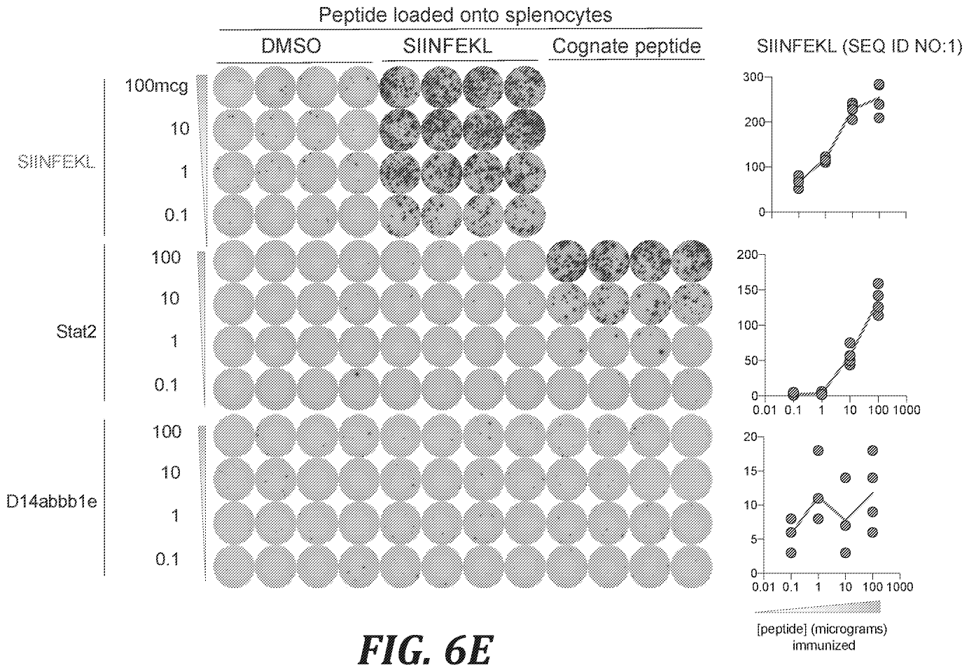

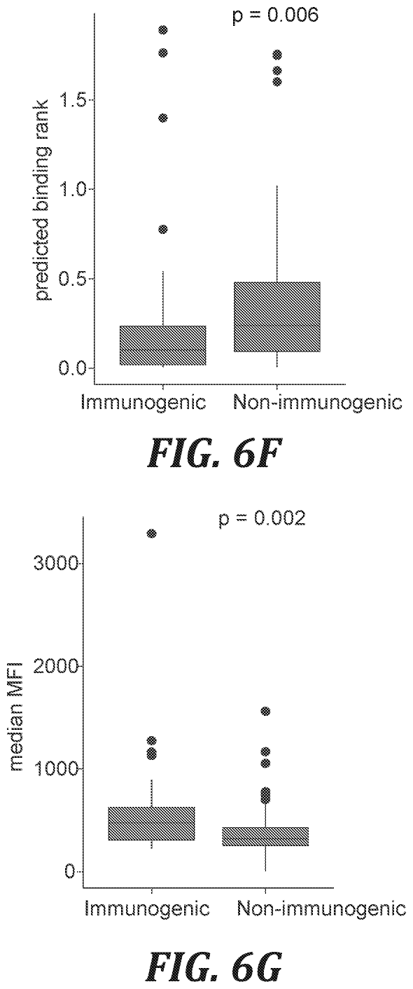

First, the ability of each of the 109 candidate neoantigenic peptides, which arose from a diversity of indisulam-induced splicing changes (FIGS. 5I-5M), to bind H-2D^{b}, H-2K^{b}, or both was validated. Each peptide was synthesized and its ability to bind MHC I was evaluated with the RMA-S stabilization assay. RMA-S cells are deficient for the transporter associated with antigen processing gene (TAP2), and therefore unable to present endogenous peptides on MHC I. These ‘empty’ MHC I molecules, either H-2D^{b} or H-2K^{b}, are unstable on the plasma membrane and usually internalized. Therefore, stable cell-surface expression of either H-2 molecule is dependent on the ability of the exogenously supplied peptide under interrogation to bind extracellularly, and thereby stabilize cell-surface H-2 molecules. This effort revealed not only that candidate peptides had a range of abilities to stabilize H-2 molecules, and therefore presumably a range of binding affinities, but also that some peptides bound efficiently to both H-2 molecules (FIGS. 5N-5Q and FIG. 6A). ˜97% (68/70) of peptides identified by intersecting MHC I mass spectrometry and RNA-seq analyses showed at least some binding to either H-2D^{b} or H-2K^{b}, and several exhibited very strong binding. In contrast, performing the same assays with negative control “spike-in” peptides that were used as non-binder decoys for mass spectrometry spectra mapping revealed no binding. Of note, these assays included well-studied, antigenic peptides known to bind H-2D^{b}, H-2K^{b}, or both (the immunodominant ovalbumin peptide SIINFEKL (SEQ ID NO:1), gp100, and Trp1 heteroclitic peptide) as comparators; a number of splicing modulator-induced candidate antigenic peptides that were identified stabilized MHC I even more efficiently than these known immunogenic antigens.

Given that MHC I binding alone is imperfectly correlated at best with immunogenicity, it was next assessed whether each candidate peptide was, in fact, immunogenic. Naïve mice were immunized with 10 μg of each of the above 109 peptides (individually) emulsified in TiterMax Classic vaccine adjuvant by bilaterally injecting into the hocks, and obtained draining lymph nodes seven days later (FIG. 6B). IFNγ ELISpot analysis of CD8⁺ T cells purified from these lymph nodes were then performed after stimulation by incubating with naïve, syngeneic splenocytes loaded with DMSO or cognate peptide. This analysis revealed that ˜43% (30 of 70) of the peptides with both RNA-seq and mass spectrometry support were able to elicit a CD8⁺ T cell response in vivo (FIGS. 6C-6D), thereby functionally verifying a subset of splicing-induced candidate neopeptides as bona fide antigens. Several of these immunogenic peptides were induced by indisulam in all tested mouse cancer cell lines. The specificity of these responses was further confirmed by performing immunization experiments across a range of peptide doses (FIG. 6E). This revealed a dose-dependent response of CD8⁺ T cells to increasing concentrations of peptides used for immunization, with some peptides eliciting an even more profound response than SIINFEKL (SEQ ID NO:1). By contrast, a non-immunogenic peptide (D14Abble) was unable to elicit effective CD8⁺ T cell responses even at the highest 100 μg dose (FIG. 6E).

Of the 39 splicing-derived, candidate immunogenic peptides identified based solely on RNA-seq analyses and MHC I binding predictions, all exhibited some degree of binding in the RMA-S assay to H-2db or H-2Kb (not shown), and 28% (11 of 39) were immunogenic in vivo (not shown). These latter results compare favorably with those based on candidate antigens identified by integrating mass spectrometry and RNA-seq data, with the caveat that the mass spectrometry-based predictions imply endogenous processing and antigen presentation, while the RNA-seq-based predictions do not. These results suggest that computational analyses of RNA-seq and predicted MHC I binding alone have the potential to identify a reasonable proportion of splicing-derived, potentially immunogenic peptides. It is also important to note, however, that a number of candidate splicing-derived, antigenic peptides with verified MHC I binding nonetheless failed to elicit activation of CD8⁺ T cells in vivo (not shown). To attempt to understand the basis for this differential response, potential features which could distinguish immunogenic versus nonimmunogenic splicing-derived peptides were interrogated. Analyses of diverse features, including predicted binding affinity (NetMHCpan 4.0), experimental ability to stabilize MHC I (RMA-S assay), parent gene expression, type and magnitude of splicing alteration, and predicted induction of NMD revealed that only the strength of binding to MHC class I (as predicted by NetMHCpan 4.0 or assayed by the RMA-S experiments) differed significantly between immunogenic versus nonimmunogenic peptides, though it remains an imperfect predictor (FIGS. 6F-6G).

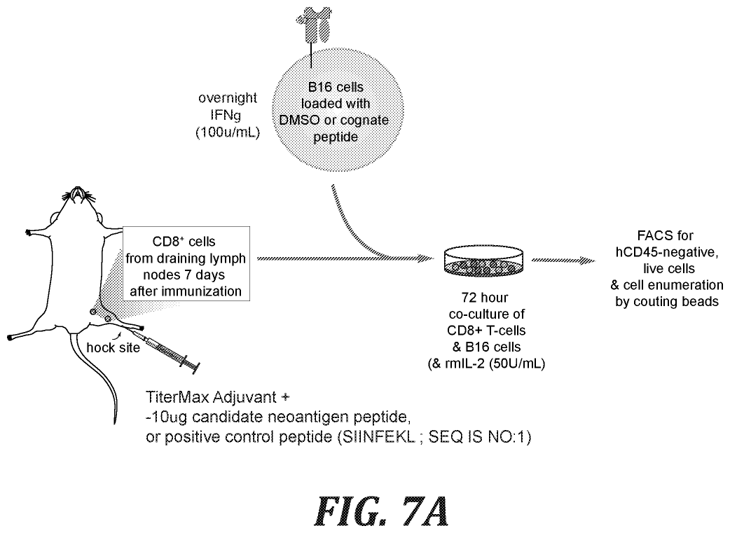

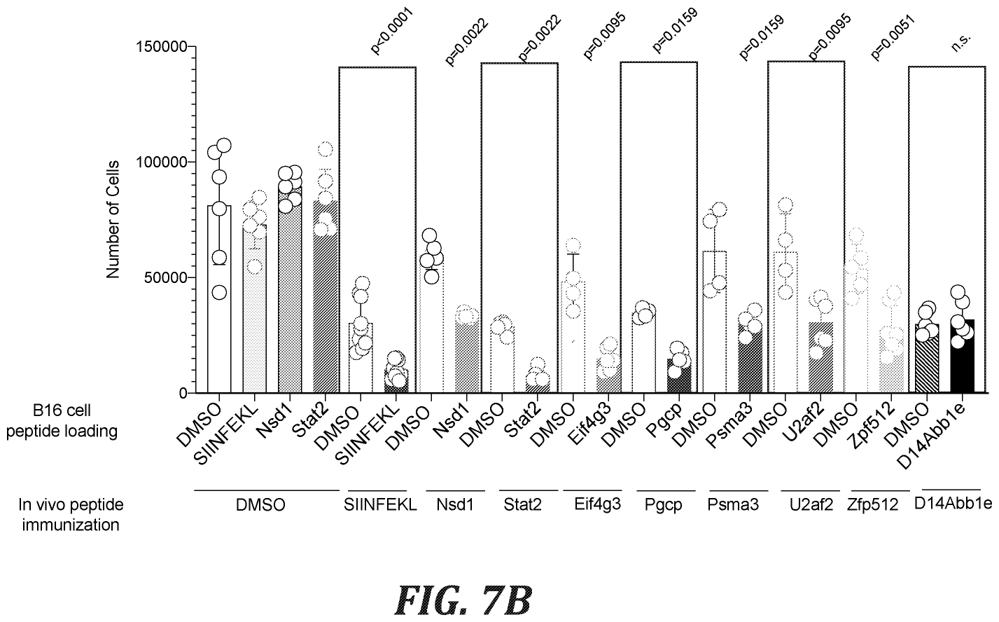

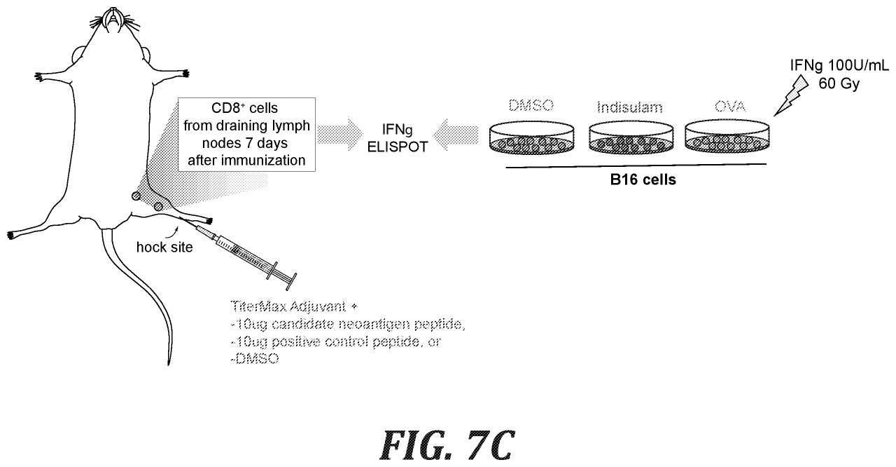

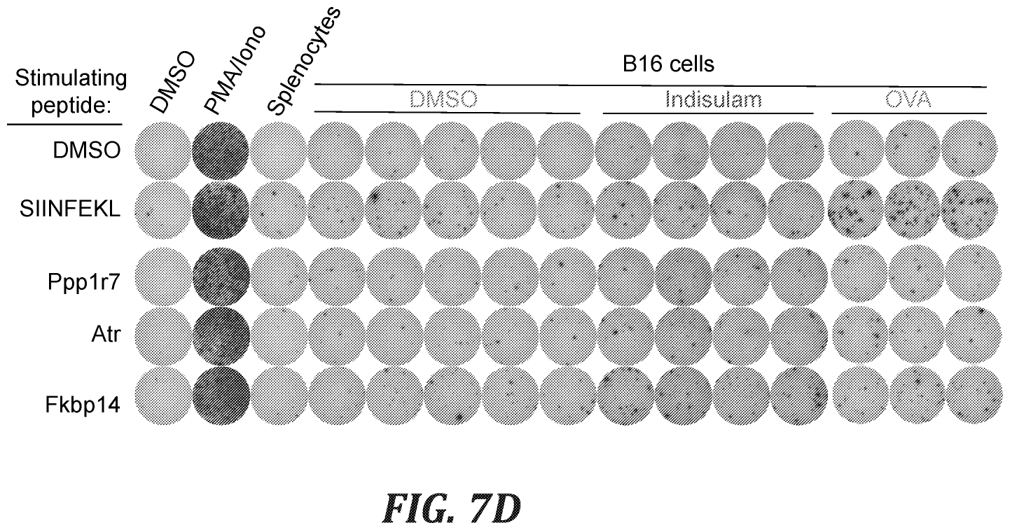

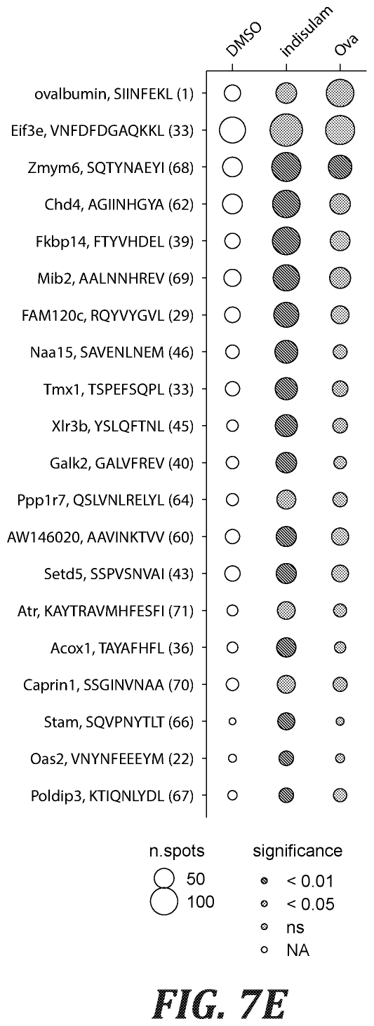

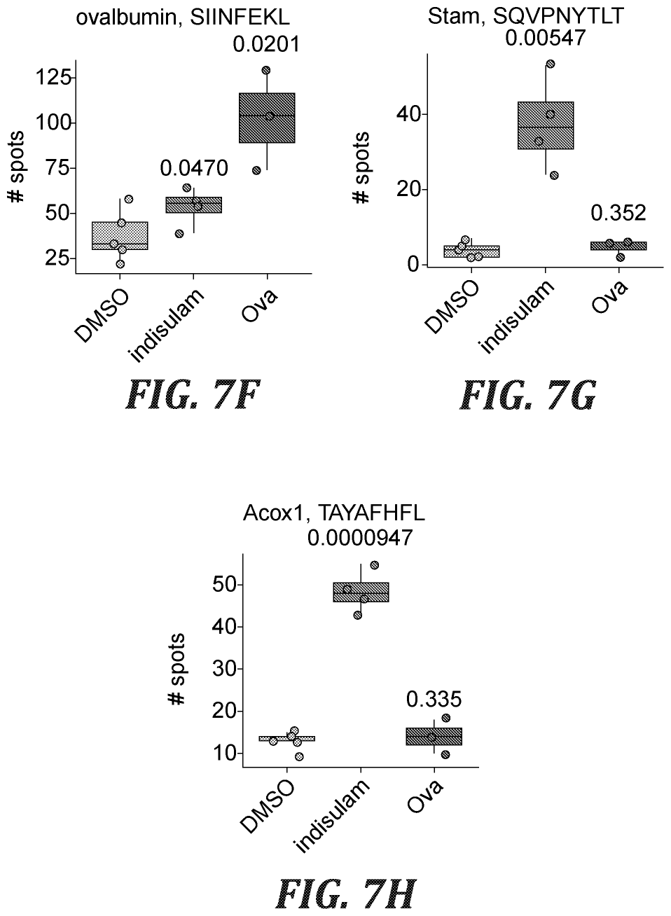

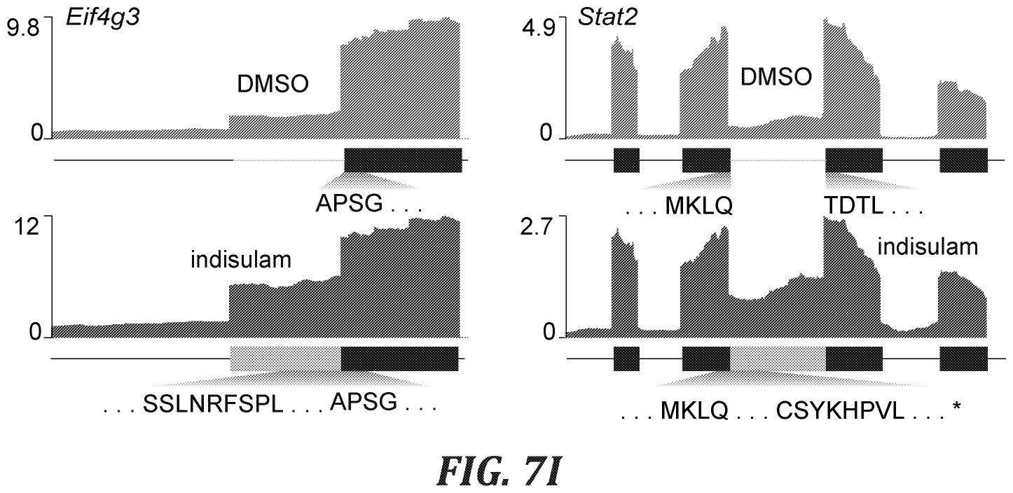

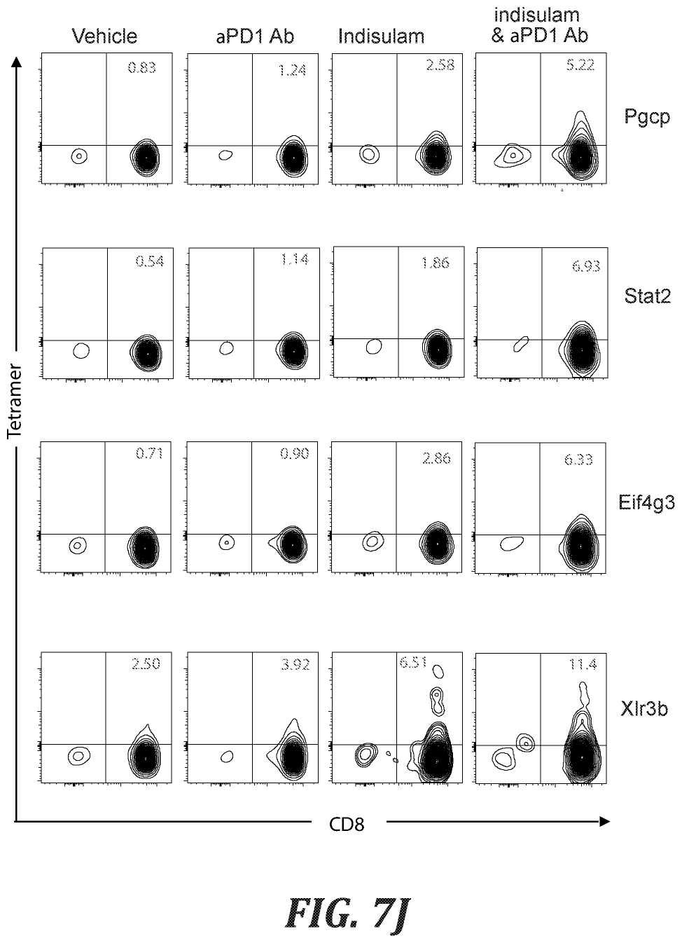

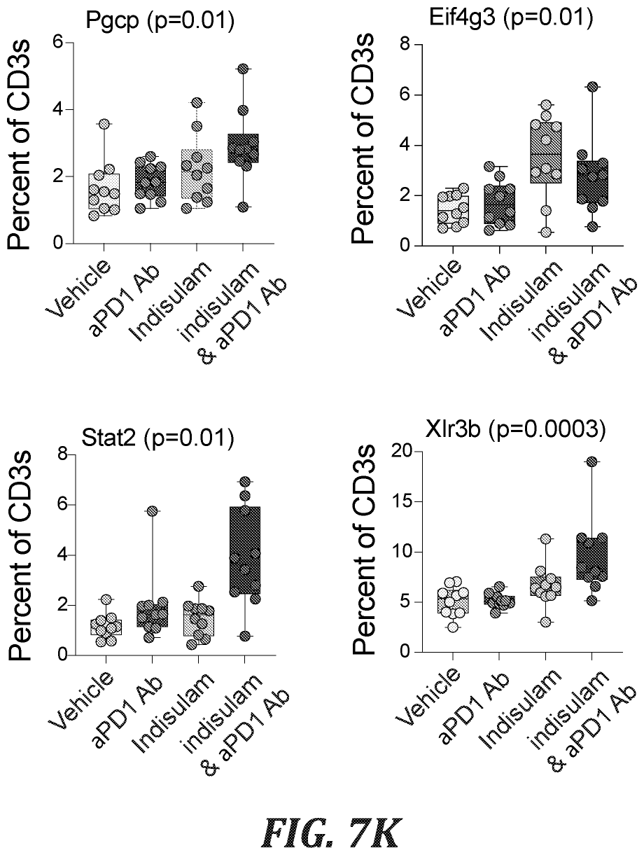

The above evaluation of the effects of candidate splicing-derived antigenic peptides on IFNγ secretion by CD8⁺ T cells was extended by testing the ability of CD8⁺ T cells from peptide-immunized mice to kill tumor cells presenting the cognate peptide (FIGS. 7A-7B). While DMSO-immunized CD8⁺ T cells exerted no cytotoxic activity regardless of the peptide presented, CD8⁺ T cells from mice immunized with an immunogenic peptide selectively killed B16-F10 cells presenting that same peptide. Conversely, CD8⁺ T cells from mice immunized with a negative control peptide (D14Abble) were unable to mediate cytotoxicity.

Next, the endogenous consequences of splicing-derived peptide production were assessed by testing whether drug-treated tumors generated neoantigenic peptides at concentrations which activated CD8⁺ T cells. The above peptide immunization experiments were repeated but instead used B16-F10 cells treated with indisulam as antigen-presenting cells (FIG. 7C). These experiments demonstrated that indisulam treatment of tumor cells indeed stimulates endogenous generation of specific splicing-derived neoantigens that triggers antigen-specific T cell activation (FIGS. 7D-7H). Given this, it was tested whether indisulam treatment drove the expansion of antigen-specific CD8⁺ T cells that recognized indisulam-promoted neoantigenic peptides in vivo (FIG. 7I). Fluorescently labeled H-2K^{b} tetramers loaded with peptides which elicited strong IFNγ secretion and cytotoxicity in the above peptide immunization experiments were generated (Xu, X. N., and Screaton, G. R. (2002). MHC/peptide tetramer-based studies of T cell function. J Immunol Methods 268, 21-28, incorporated herein by reference in its entirety). These tetramers were then used to stain tumor-draining lymph nodes (DLN) of mice bearing B16-F10 tumors treated with vehicle, indisulam, anti-PD1, or the combination (FIG. 7J). This revealed increased frequencies of CD8⁺ T cells bearing TCRs capable of recognizing these splicing-derived peptides in the tumor DLN of mice receiving indisulam or the combination of indisulam and anti-PD1, consistent with an expansion of reactive T cells upon the promotion of neoantigen generation by indisulam (FIG. 7K). Together, these data demonstrate that splicing inhibition triggers the production of specific splicing-derived neoantigens at levels sufficient to drive expansion of CD8⁺ T cells recognizing those antigens.

### Discussion

Prior work has identified individual examples of antigenic peptides that can arise from gene products beyond unannotated peptide sequences. These include the generation of individual neoantigens via gene fusions, aberrant splicing, translation of alternative open reading frames, RNA editing, peptide splicing, and post-translational modifications of proteins. However, the potential for acutely inducing such neoantigens for therapeutic purposes has not been demonstrated previously. This work demonstrates that multiple clinical grade, splicing inhibitory compounds, acting via unrelated mechanisms of action, enhance the activity of immune checkpoint blockade via the generation of aberrantly spliced mRNAs encoding antigenic peptides presented on MHC I. At therapeutic doses in vivo, pharmacologic splicing modulation enhanced anti-tumor T cell immunity. These studies thereby identify a strategy to acutely and reversibly induce tumor neoantigens without changes at the genomic level, and additionally extend prior in silico predictions of the potential for splicing-derived neoepitopes by directly demonstrating their antigenic potential and functional relevance in vivo.

Anti-cancer aryl sulfonamide compounds such as indisulam, E7820, and chloroquinoxaline sulfonamide have been shown to function selectively via on-target degradation of RBM39, as point mutations in RBM39 or deletion of the ubiquitin ligase adaptor DCAF15 rescue all of the cellular effects of these compounds. In contrast to indisulam and other RBM39 degraders, Type I PRMT enzymes have numerous cellular substrates, and inhibition of these enzymes has pleotropic effects. Despite this, RNA-binding proteins and splicing factors represent the largest proportion of cellular substrates of PRMT enzymes according to multiple methylarginine proteomic studies. Consistent with these findings, despite the potential protean nature of Type I PRMT inhibitors, their effects in our studies were ascribable to MHC I-presented peptides. Furthermore, while each of the therapeutic modalities studied here perturbed splicing in both tumoral and non-tumoral cells, no immune-related adverse effects were identified in combining splicing modulation with immune checkpoint blockade. Whether or not such immune-related toxicities might be encountered in a clinical setting remains to be evaluated.

Although many factors regulate response to checkpoint immunotherapy, neoantigen burden is an important determinant of response, as evidenced by the success of checkpoint immunotherapy in mismatch repair-deficient and POLD1/POLE-mutated cancers. Recent studies have highlighted that the increased presence of frameshift insertion/deletion mutations in these genetic subtypes of cancer are a particularly highly immunogenic subset of somatic variants. Here, an analogously abundant source of highly immunogenic peptides derived from novel mRNA species arising from pharmacologic splicing modulation is identified. Despite the potential for such incompletely spliced mRNAs to undergo nuclear retention and/or nonsense-mediated decay (NMD), sequencing of cytoplasmic pools of mRNA combined with direct mass spectrometric evaluation definitively identified a subset of these peptides as present in the cytoplasm and translated into MHC I-bound peptides.

Rather than causing relatively small changes in amino acid sequence, as seen with single nucleotide variants (SNVs), modulation of RNA splicing generates many novel mRNA species derived from large-scale events, including inclusion of intronic regions into mature mRNA, juxtaposition of exons not normally spliced together, and generation of exons with abnormal 5′ or 3′ ends. Each of these processes can result in the downstream translation of many peptides containing wholly novel sequences, potentially contributing to the large number of immunogenic peptides that were identified in vivo. While direct comparisons of the frequencies of neoantigens derived from aberrant splicing to those derived from SNVs is challenging due to differences in how immunogenicity is measured and how candidate peptides were determined, the frequency of antigenic peptides derived from splicing may be unexpectedly high. For example, following stringent selection of candidate neoantigenic peptides derived by intersecting RNA-seq and MHC I proteomic data, it was found that 30/70 (˜43%) tested splicing-derived novel peptides could elicit a CD8⁺ T cell immune response in naïve C57BL/6 mice (as measured by IFNγ ELISpot). Predicted neoantigenic peptides derived from RNA-seq data alone exhibited a positivity rate of 11/39 (˜28%). Of these experimentally-confirmed neoantigenic peptides, it was demonstrated that four were associated with the expansion of antigen-specific CD8⁺ T cells recognizing those specific neoantigens following splicing modulator drug treatment of tumor-bearing mice. In comparison, an early seminal study of MC38 cells identified that out of ˜1,300 coding variations, ˜13% resulted in peptides predicted to bind MHC I, 0.5% of which were identified by mass spectrometry, and ˜0.25% of which were immunogenic in vivo as defined by tetramer staining and vaccination experiments (Yadav, M., et al. (2014). Predicting immunogenic tumour mutations by combining mass spectrometry and exome sequencing. Nature 515, 572-576, incorporated herein by reference in its entirety). Similarly, in the d42m1-T3 sarcoma cell line, investigators calculated 93,892 predicted candidate neoantigens, then narrowed this list to 66 predicted strong binders to H-2K^{b} or H-2D^{b}, of which two (3%) were both evident in mass spectrometry as well as immunogenic in vivo as measured by tetramer staining and IFNγ ELISpot (Gubin, M. M., et al. (2014). Checkpoint blockade cancer immunotherapy targets tumour-specific mutant antigens. Nature 515, 577-581, incorporated herein by reference in its entirety). Previous studies of primary human cancers have reported similar percentages of immunogenic neopeptides. For example, in human gastric cancer, a study inferring neoantigens from whole exome/genome sequencing data found 38 to 264 mutations per patient, with correspondingly one to three bona fide immunogenic neoepitopes by IFNγ ELISpot (Tran, E., et al. (2015). Immunogenicity of somatic mutations in human gastrointestinal cancers. Science 350, 1387-1390, incorporated herein by reference in its entirety). Finally, a recent consortium effort evaluating human melanoma and non-small cell lung cancer neoantigens predicted to bind MHC also reported a relatively similar immunogenicity rate of 6% of candidate peptides by peptide:MHC multimer studies (Wells, D. K., et al. (2020). Key Parameters of Tumor Epitope Immunogenicity Revealed Through a Consortium Approach Improve Neoantigen Prediction. Cell 183, 818-834 e813, incorporated herein by reference in its entirety).

A large number of splicing-derived, potentially immunogenic peptides that are produced upon exposure to splicing inhibitors were identified, at least some of which trigger reactive T cell expansion in vivo. Based on the diverse and large set of immunogenic peptides induced by perturbing splicing, it was hypothesized that multiple peptides can contribute to tumor control following splicing modulatory therapy. Thus, it is likely more difficult for tumors to escape immune control induced by splicing modulation than to escape mutational antigen-dependent control, which often appears to arise from just one or a few antigens. This lowered potential for cancers to escape the inhibitory effects demonstrated by the disclosed combination therapy incorporating RNA splicing modulating agent(s) in coordination with immunotherapies, such as immune checkpoint inhibitor(s).

### Methods

Mice

All in vivo experiments were approved by the Institutional Animal Care and Use Committees (IACUC) of Memorial Sloan-Kettering Cancer Center and/or Fred Hutchinson Cancer Research Center. All animals were housed in the respective specific pathogen free (SPF) barrier facilities and maintained under standard husbandry conditions. B6(Cg)-Rag2tm1.1Cgn/J (RA2 KO) mice, C57BL/6-Tg(TcraTcrb)1100Mjb/J (OT-1) mice and B6.129P2-B2mtm1Unc/DcrJ (B2M KO) mice were obtained from Jackson Laboratories (Cat. 008440, 003831, 002087 respectively). C57BL/6 mice, congenic B6.SJL-Ptprc^{a} Pepc^{b}/BoyJ (CD45.1) mice, Balb/c and LP/J mice were also obtained from Jackson Laboratories (Cat. 000664, 002014, 000651 and 000676).

Cell Lines

B16-F10, CT26.WT (CT26), and LLC cells were obtained from ATCC (Cat. CRL-6475, CRL-2638, and CRL-1642 respectively). MB49 cells were obtained from MilliporeSigma (Cat. SCC148, Burlington, Mass.); MC38 cells were obtained from Kerafast (Cat. ENH204-FP, Boston, Mass.). B16-F10 and MC38 cells expressing chicken ovalbumin (B16ova and MC38ova) were a kind gift of Jeff Ravetch (Rockefeller University, New York, N.Y.). To generate β₂ microglobulin deficient cell lines for in vitro experiments, four candidate sgRNAs for mouse β₂microglobulin (#1 AGTATACTCACGCCACCCACCGG (SEQ ID NO:2), #2 TCACGCCACCCACCGGAGAATGG (SEQ ID NO:3), #3 GGCGTATGTATCAGTCTCAGTGG (SEQ ID NO:4), #4 TCGGCTTCCCATTCTCCGGTGGG) (SEQ ID NO:5), or nontargeting control (GGAGCGCACCATCTTCTTCA) (SEQ ID NO:6) were cloned into pSpCas9(BB)-2A-Puro (PX459) as previously described (Ran, F. A., et al. (2013). Genome engineering using the CRISPR-Cas9 system. Nat Protoc 8, 2281-2308, incorporated herein by reference in its entirety) and used to engineer deficient MC38, B16-F10, and CT26 cell lines via transfection using XtremeGene 9 reagent as per manufacturer's instructions (MilliporeSigma Cat. 6365809001) followed by puromycin selection at 10 μg/mL for three days. Polyclonal cell populations were obtained by flow sorting for H-2K^{b}/D^{b} and P2 microglobulin double-negative cells, and gene knockout further confirmed by stimulating a culture of these sorted cells for 48 hours with 10 U/mL mouse IFNγ and analyzing for the same markers. For in vivo experiments, lentiCas9-Blast was used to generate Cas9-expressing B16-F10 cells. B2m gRNAs (GAGGGGTTTCTGAGGGCCAC (SEQ ID NO:7), AGTATACTCACGCCACCCAC (SEQ ID NO:8)) and non-targeting control gRNAs (AAAAAGTCCGCGATTACGTC (SEQ ID NO:9), ACCCATCCCCGCGTCCGAGA (SEQ ID NO:10)) were cloned into lentiGuide-Puro and introduced into Cas9-expressing B16-F10 cells via lentiviral transduction as previously described (Thomas, J. D., et al. (2020). RNA isoform screens uncover the essentiality and tumor-suppressor activity of ultraconserved poison exons. Nat Genet 52, 84-94, incorporated herein by reference in its entirety) and underwent similar selection. PX459, lentiCas9-blast, and lentiGuide-Puro are available from Addgene Cat. 62988, 52962, 52963, respectively.

Pretreatment with Splicing Inhibitors

Unless otherwise specified, cell lines were treated with splicing inhibitors at the indicated concentrations for 96 hours in vitro, harvested and washed three times with PBS in excess to remove all drug, and then used for downstream analyses and/or subsequent studies, including phenotyping, RNA-seq analyses, continued growth in vitro, or tumor challenge in vivo into syngeneic animals.

In Vivo Tumor Challenge

Unless otherwise specified, syngeneic B6 or Balb/c mice were engrafted subcutaneously on bilateral flanks with MC38, B16-F10, CT26 or LLC tumor cells at the following doses: MC38 10⁶ cells, B16-F10 0.5×10⁶ cells, CT26 0.25×10⁶ cells, LLC 0.25×10⁶ cells. Tumors were measured serially twice or three times weekly and tumor volumes were estimated by length×width×height. Animals were monitored daily for survival and weighed twice weekly. Experimental endpoints mandating euthanasia were approved by the IACUC and included: animal lethargy, severe kyphosis or evidence of pain, difficulty with ambulation or feeding, tumor ulceration>1 cm or bleeding tumor, evidence of infected tumor, tumor volumes exceeding 2.5 cm³, or animal total body weight loss>10% from baseline.

Determination of Cell Growth, Annexin V, and Activation Marker IC₅₀ Values

Cell lines were grown with half-log₁₀ concentrations of the indicated drug in 4 to 8 technical replicates under standard conditions until the control condition (DMSO or vehicle) was confluent by microscopy. For tumor cell lines, viable cells were quantified via the CellTiter-Glo® assay (Promega Cat. G7573) as per manufacturer instructions. For the ex vivo proliferation of T cells, viable cells were instead quantified via flow cytometry using counting beads. The percentage or number of viable cells with drug treatment was calculated relative to DMSO control (as 100%). These data were log₁₀ transformed and a three-parameter nonlinear fit of log(inhibitor) vs. response was performed in GraphPad Prism v9.0 (GraphPad Software, San Diego, Calif.) to determine IC₅₀ values. For absolute cell number, Annexin V+, CD25+, and PD1+ flow cytometry data presented in Figure S4, dose-response models and IC₅₀ values were computed using the R language's dre package (Ritz, C., et al. (2015). Dose-Response Analysis Using R. PLoS One 10, e0146021).

OT-1 Cytotoxicity Assay

Bulk splenocytes from OT-1 animals were cultured for three days with 100 U/mL murine IL-2 and 100 μg/mL SIINFEKL (SEQ ID NO:1) peptide to activate CD8⁺ T cells. Cultures were subsequently washed thoroughly to remove ova peptide and rested for at least 24 hours prior to use. OT-1 cells were passaged in T cell media with 50 U/mL IL-2 for no more than seven days from animal sacrifice prior to use. For the cytotoxicity assay, tumor cells alone or OT-1+tumor cells (1:1 ratio) were incubated in T cell media for 18 hours under standard conditions with the indicated concentrations of splicing drugs and analyzed by flow cytometry to quantify killing. OT-1 cells and other hematopoietic cells were excluded with the use of CD45, CD3, and CD8 staining. Tumor cell viability was measured using DAPI.

LAMP-1 T Cell Degranulation Assay

OT-1 cells were generated as described for the cytotoxicity assay and incubated with ovalbumin-expressing tumor cell lines (pre-treated overnight with IFNγ 100 U/mL to upregulate cell-surface MHC I) in the presence of DMSO or varying concentrations of splicing modulator drugs as indicated, in the presence of LAMP-1 antibody for 5-6 hours under standard incubator conditions. After the first hour of incubation, BD GolgiPlug™ (brefeldin A) and BD GolgiStop™ (monensin) was added at 1:1,1000+1:1,500 respectively into cells. At the end of incubation, cells were washed and stained for cell surface markers prior to standard flow cytometry.

Generation and Use of Peptide:H-2K^{B} Tetramers

Peptide:MHC I tetramers with neoantigenic peptides and murine H-2K^{b} were generated using the QuickSwitch™ Quant Tetramer Kit-PE (Cat. TB-7400-K1, MBL International) per manufacturer instructions. Briefly, 10 μg of peptide together with 50 μL of the tetramer reagent and 1 μL of peptide exchange factor were incubated at room temperature for 5-6 hours and used to stain cell populations of interest. Clone KT15 of an anti-CD8 antibody (Cat. D271-A64, MBL International) was used to identify CD8⁺ T cells of interest as this clone does not interfere with tetramer binding.

Intracellular Cytokine Staining

OT-1 cells were prepared and incubated with ovalbumin-expressing tumors as described above in the LAMP-1 assay. For some experiments OT-1 cells were instead left unstimulated (DMSO) or treated with PMA 1 μg/mL+ionomycin 1 μM as a supraphysiologic stimulus. In all cases, T cells underwent a 5-6 hour incubation period in the presence of DMSO or splicing modulators at the indicated concentrations, and with brefeldin A and monensin present for the entire duration. Cells were subsequently washed, stained for surface markers, and then fixed/permeabilized for intracellular staining of the indicated cytokines according to manufacturer instructions (BD Biosciences).

Western Blotting

Western blotting was performed as per standard techniques. Anti-RBM39 (Atlas Antibodies, Cat. HPA001591 or Bethyl laboratories, Cat. A300-291A) were used to detect RBM39 degradation. ADMA and SDMA levels were determined using antibodies from Cell Signaling Technologies (Cat. 13222S and Cat. 135225). Actin antibody (clone AC-15) was obtained from MilliporeSigma (Cat. A5441-.2ML). Densitometry of RBM39 and actin loading control was performed using ImageJ software in order to calculate RBM39 degradation IC₅₀ values.

Therapeutic Treatment with Splicing Compounds and Anti-PD1

Animals were subcutaneously engrafted on bilateral flanks with tumor cells (MC38 1×10⁶, B16-F10 0.5×10⁶ and LLC 0.25×10⁶ cells unless otherwise specified) on day 0, and treated continuously with splicing inhibitors (MS-023 50 mg/kg i.p., indisulam 25 mg/kg i.v. or vehicle) daily for 5 of 7 weekly days starting from day +3 of tumor challenge. Indisulam was obtained from MilliporeSigma (Cat. SML1225-25MG) and MS-023 in sufficient quantities for in vivo studies was synthesized by the authors as previously described (Eram, M. S., et al. (2016). A Potent, Selective, and Cell-Active Inhibitor of Human Type I Protein Arginine Methyltransferases. ACS Chem Biol 11, 772-781, incorporated herein by reference in its entirety). For in vivo formulation, indisulam was dissolved in sterile DMSO at 50 mg/mL and this was combined in a 1:20 ratio with 15% 2-Hydroxypropyl-β-cyclodextrin (Sigma. Cat. H107-100G) in sterile water (w/v) and filtered through a 0.45 μM filter to yield a final solution of 2.5 mg/mL. For in vivo formulation, 62.5 mg of MS-023 was dissolved in 563 microliters of 1-methyl-2-pyrrolidinone (NMP, Sigma. 328634-1L), diluted with 2.257 mL of 20% Captisol in sterile water (w/v, SelleckChem Cat. S4592) and further combined with 2.257 mg of polyethylene glycol 400 (PEG-400, Sigma Cat. PX1286B-2), and 6.21 mL of PBS, mixed by vortexing and sterile filtered to yield a solution of 5.5 mg/mL. Mice were weighed weekly for weight-based drug dosing. Animals were treated with 250 μg of anti-PD1 flat dose (clone RMP1-14, BioXCell Cat. BE0146) or PBS i.p. starting on day +7 and twice weekly thereafter for a total of five doses.

In Vivo T Cell or NK Cell Depletion

For depleting T cells, mice were treated with simultaneous anti-CD4 (clone GK1.5, BioXCell Cat. BE0003-1) together with anti-CD8 (clone 2.43, BioXCell Cat. BE0061) versus PBS control, at days −7, −4, +4, and +7 relative to tumor challenge on day 0. Each depleting antibody was administered i.p. at 0.5 mg per dose. 0.5×10⁶ B16-F10 which were treated in vitro with indisulam at 1 μM or DMSO for 96 hours were engrafted subcutaneously on the flanks of animals receiving T cell depletion or PBS control. For NK cell depletion, an identical experimental schedule and dose using clone PK136 (BioXCell Cat. BE0036) was utilized. To verify T cell depletion, CD4 clone H129.19 (Biolegend Cat. 130310), CD8 clone 53-5.8 (Biolegend Cat. 140410) were used. NKp46 (Biolegend Cat. 137608) was used to verify NK cell depletion.

CFSE Adoptive T Cell Transfer and Splicing Modulator Treatment

Splenic T cells were obtained from B6 or CD45.1 donors by CD5 positive selection (Miltenyi Biotec, Cat. 130-049-301), labeled with CellTrace CFSE (ThermoFisher Cat. C34570) at 10 μM, and adoptively transferred by tail vein injection into lethally irradiated B6, Balb/c, or LP/J recipients, with 107 labeled donor T cells transferred per recipient. All recipients were irradiated on day −1 prior to adoptive T cell transfer with 7 Gy as a single fraction and continuously received splicing inhibitor drugs or vehicle control at the indicated doses, from day −1 until day of sacrifice, with the initial dose of drug at least 4 hours after lethal irradiation. Indisulam and MS-023 were solubilized for in vivo administration and animals were treated as above. Pladienolide B (Tocris, Cat. 6070) and GEX1A (Cayman Chemicals, Cat. 25136) were both dissolved in vehicle (10% ethanol and 4% Tween-80 in sterile PBS) and administered i.p., with pladienolide B dosed at 10 mg/kg every other day, and GEX1A dosed at 1.25 mg/kg every four days. For in vivo use, EPZ015666 was dissolved in DMSO and solubilized in 0.5% methylcellulose in water to 20 mg/mL; animals were treated daily with 200 mg/kg by oral gavage.

Anti-CD3/CD28 T Cell Activation

Plates were coated with 10 μg/mL anti-CD3 (clone 145-2C11, Biolegend Cat. 100302) and 2 μg/mL anti-CD28 (clone 37.51, Biolegend Cat. 102102) in PBS overnight at 4° C. and washed twice with cold PBS prior to use. CFSE-labeled CD5-selected splenic T cells from naïve C57BL/6J mice were obtained identically as for adoptive cell transfer, and 5×10⁴ cells incubated with coated plates in the presence of splicing inhibitor drugs at the indicated concentrations, followed by analysis by standard flow cytometry on day 3. Of note, for RNA-seq analyses, T cells were not labeled with CFSE, and underwent activation for 4 days (96 hours) in the presence of various splicing modulator drugs to harmonize experimental conditions with RNA-seq analyses of tumors treated with splicing inhibitors. For the RNA-seq experiments only, T cells in all conditions were also incubated with IL-2 at 50 U/mL to maximize viability and yield.

Mixed Leukocyte Reaction

RBC lysed bone marrow obtained from the femurs and tibias of C57BL/6 or P2 microglobulin deficient mice (Jackson Laboratories Cat. 2087) were cultured with mouse IL-3 (PeproTech Cat. 213-13) and mouse FLT3 ligand (PeproTech Cat. 250-31L) both at 10 ng/mL each in RPMI+10% FCS for 7 days to generate bone marrow derived dendritic cells. Separately, 107 MC38 treated with splicing inhibitors vs. DMSO or expressing chicken ovalbumin were harvested, washed and resuspended in sterile PBS, and subjected to five cycles of rapid freeze-thaw (alternating between 37° C. and dry ice/acetone) to generate a cell lysate. After brief centrifugation at 100×g, the soluble fraction in PBS was added to bone marrow derived DCs and left to incubate overnight for antigen phagocytosis in the presence of LPS (ThermoFisher Cat. 00-4976-93). DCs were subsequently washed three times to remove cell-free lysates and LPS and incubated in a 1:1 ratio with CFSE-labeled B6 splenic T cells (10⁵ stimulators with 10⁵ responders) as described above. The MLR was analyzed at day 5 by flow cytometry.

M3434 Methylcellulose Colony Assay

25,000 red blood cell-lysed bone marrow mononuclear cells from C57BL/6 mice were plated in duplicates or triplicates in each well of a non-tissue-culture treated 6 well plate with M3434 methylcellulose media in the presence of splicing drugs at the indicated concentrations as per manufacturer's instructions (StemCell Technologies, Cat. 03434) and incubated for seven days prior to quantification of colonies by manual microscopy.

Intracellular Flow Cytometry

Cells were fixed with 2.1% formaldehyde in PBS for 10 minutes at 37° C., washed and permeabilized with ice-cold 90% methanol for 30 minutes, and washed prior to staining. If required, cell surface staining was performed after fixation but prior to permeabilization. For some experiments, intracellular staining was performed using the eBioscience™ Foxp3 transcription factor staining buffer set (ThermoFisher Cat. 00-5523-00) or reagents for intracellular cytokine staining (BD Cytofix/Cytoperm™, Cat. 554714, and BD Perm/Wash™, Cat. 554723) as per manufacturer's instructions.

Histology

Animal tissues were fixed in 4% paraformaldehyde, decalcified (for bone), dehydrated and paraffin embedded. Blocks were sectioned and stained with hematoxylin and eosin or anti-CD8. Images were acquired using an Axio Observer A1 microscope (Carl Zeiss, Oberkochen, Germany) or scanned using an Aperio AT slide scanner (Leica Biosystems, Buffalo Grove, Ill.). Automated quantification of infiltrating CD8⁺ T cells was performed using HALO software (Indica labs, Albuquerque, N. Mex.). Pathologic evaluation of immune-related tissue toxicities was performed in a blinded fashion by one of the authors who is a trained pathologist (Ben Durham, MD).

Cellular Fractionation for RNA Sequencing

Nuclear and cytoplasmic cellular fractions were isolated from B16-F10 cells using reagents from Active Motif (Cat. 25501) as per manufacturers' instructions, with the exception of RNA isolation and purification from each fraction using the QIAgen RNeasy Mini kit.

RNA Sequencing

Bulk lung and colon were homogenized using a Qiagen TissueRuptor. For all tissues and cell types, RNA was extracted using an RNeasy kit (Qiagen, Frederick, Md.) and quantified using a NanoDrop 8000 (ThermoFisher Scientific). A minimum of 500 ng of high-quality RNA (as determined by Agilent Bioanalyzer) per sample or replicate was used for library preparation. Poly(A)-selected, strand-specific (dUTP method) Illumina libraries were prepared with a modified TruSeq protocol and sequenced on the Illumina HiSeq 2000 (˜100M 2×101 bp paired-end reads per sample or replicate).

Data Availability

All RNA-seq data generated as part of this study were deposited at the Gene Expression Omnibus (accession GSE162818).

RNA-Seg Data Analysis

RNA-seq analysis was performed as previously described (Dvinge, H., et al. (2014). Sample processing obscures cancer-specific alterations in leukemic transcriptomes. Proc Natl Acad Sci USA 111, 16802-16807, incorporated herein by reference in its entirety). Briefly, FASTQ files were mapped using RSEM version 1.2.4 (Li, B., and Dewey, C. N. (2011). RSEM: accurate transcript quantification from RNA-Seq data with or without a reference genome. BMC Bioinformatics 12, 323, incorporated herein by reference in its entirety) (modified to call Bowtie (Langmead, B., et al. (2009). Ultrafast and memory-efficient alignment of short DNA sequences to the human genome. Genome Biol 10, R25, incorporated herein by reference in its entirety) with option ‘-v 2’) to mouse or human transcriptome annotations built using transcript information from Ensembl v71.1 (Flicek, P., et al. (2013). Ensembl 2013. Nucleic Acids Res 41, D48-55, incorporated herein by reference in its entirety), UCSC knownGene (Meyer, L. R., et al. (2013). The UCSC Genome Browser database: extensions and updates 2013. Nucleic Acids Res 41, D64-69), and MISO v2.0 (Katz, Y., et al. (2010). Analysis and design of RNA sequencing experiments for identifying isoform regulation. Nat Methods 7, 1009-1015). Reads that did not align at this step were then mapped using TopHat version 2.0.8b (Trapnell, C., et al. (2009). TopHat: discovering splice junctions with RNA-Seq. Bioinformatics 25, 1105-1111, incorporated herein by reference in its entirety) to the mouse (GRCm38/mm10) or human (GRCh37/hg19) genome assemblies, as well as to a database of annotated splice junctions as well as all possible new junctions consisting of linkage between each co-linear annotated 5′ and 3′ splice sites within individual genes. Aligned reads from these two mapping steps were merged to generate final BAM files for all subsequent analyses.

Gene expression estimates were computed using RSEM (performed concordantly with the RNA-seq read mapping procedure described above). Significantly differentially expressed genes were defined as those meeting the follow criteria: minimum expression of 1 transcript per million (TPM); minimum fold-change of 1.5 (log ₂ scale); p≤0.05 (computed using an unpaired, two-sided t-test comparing replicate groups for a given treatment and cell line) or a minimum Bayes factor of 100 (computed using Wagenmakers's Bayesian framework (Wagenmakers, E. J., et al. (2010). Bayesian hypothesis testing for psychologists: a tutorial on the Savage-Dickey method. Cogn Psychol 60, 158-189, incorporated herein by reference in its entirety) for the median of gene expression and associated read counts over replicates for a given treatment and cell line). Splice junction-spanning reads were filtered to require a minimum overhang of 6 nt.

MISO v2.0 was used to quantify all expression of isoforms arising from exon skipping (cassette exons), competing 5′ splice site selection, competing 3′ splice site selection, and annotated intron retention. Quantification of constitutive intron retention, where constitutive introns were defined as those whose 5′ and 3′ splice sites were never joined to other splice sites in the knownGene annotation, was calculated as previously described (Hubert, C. G., et al. (2013). Genome-wide RNAi screens in human brain tumor isolates reveal a novel viability requirement for PHF5A. Genes Dev 27, 1032-1045, incorporated herein by reference in its entirety) using reads with a minimum of 6 nt overhang in both the exon and intron. Events were considered significantly differentially spliced if they met the following criteria: a minimum of 20 identifying reads (reads which align only to one, but not both, isoforms constituting a given splicing event) in each sample; a minimum of 10% change (absolute scale) in isoform ratio or minimum fold-change of 2 (log ₂ scale) in absolute isoform ratio; p≤0.05 (computed using an unpaired, two-sided t-test comparing replicate groups for a given treatment and cell line) or a minimum Bayes factor of 5 (computed using Wagenmakers's Bayesian framework (Wagenmakers, E. J., et al. (2010). Cogn Psychol 60, 158-189, incorporated herein by reference in its entirety) for the median of isoform ratios and distinguishing read counts over replicates for a given treatment and cell line). All data parsing, statistical analyses, and data visualization were performed using the R programming environment with Bioconductor (Huber, W., et al. (2015). Orchestrating high-throughput genomic analysis with Bioconductor. Nat Methods 12, 115-121, incorporated herein by reference in its entirety).

MHC I Immunoprecipitation, Peptide Purification, and Mass Spectrometry

Peptide-MHC complexes were isolated as previously described (Abelin, J. G., et al. (2017). Mass Spectrometry Profiling of HLA-Associated Peptidomes in Mono-allelic Cells Enables More Accurate Epitope Prediction. Immunity 46, 315-326, incorporated herein by reference in its entirety), with the following modifications: anti-mouse H-2D^{b} (clone B22-249.R1, CedarLane laboratories, Cat. CL9001AP) or H-2K^{b} (clone Y-3, BioXCell Cat. BE0172) non-covalently linked to GammaBind Plus Sepharose beads were co-incubated with soluble lysates overnight. After washing with lysis buffer twice, 10 mM Tris pH 8 twice, and dH₂O twice, the peptides were desalted on C18 StaGE tips (Ishihama, Y., et al. (2006). Modular stop and go extraction tips with stacked disks for parallel and multidimensional Peptide fractionation in proteomics. J Proteome Res 5, 988-994, incorporated herein by reference in its entirety) (Pierce, Cat. 87784) and eluted using a 20%-35%-50% acetonitrile stepwise gradient. Eluted fractions were dried using a SpeedVac™ vacuum concentrator and stored until mass spectrometry. For B16-F10, cells in all experimental conditions were treated with 10 U/mL mouse IFNγ (PeproTech Cat. 315-05) for 48 hours prior to cell harvest and immunoprecipitation to upregulate surface MHC I expression.

Mass Spectrometry

Desalted, dried samples enriched for MHC peptides were resolubilized in 8 uL 0.10% TFA and 3 uL were loaded onto a packed-in-emitter 12 cm/75 um ID/3 um C18 particles column (Nikkyo Technos Co., Ltd. Japan). Peptides were eluted using a gradient delivered at 300 nL/min increasing from 2% Buffer B (0.1% formic acid in 80% acetonitrile)/99% Buffer A (0.1% formic acid) to 30% Buffer B/70% Buffer A, over 70 minutes (EasyLC 1200, Thermo Scientific). All solvents were LCMS grade (Optima, Fisher Scientific). MS and MS/MS (HCD type fragmentation) experiments were performed in data dependent mode with lock mass (m/z 445.12003) using Fusion Lumos (Thermo Scientific). Precursor mass spectra were recorded from m/z 300-1500 m/z range at 60,000 resolution. 1, 2 and 3 positive charges were selected for fragmentation experiments. MS/MS spectra were recorded at 30,000 resolution and lowest mass set at m/z 110. For MS/MS acquisition, injection time was set to maximum 100 milliseconds with an Auto Gain Control setting of 5e4. Normalized collision energy was set to 30. All experiments were recorded in FT-mode.

Proteome Creation

Gene and isoform annotations were created as described in RNA-seq data analysis. This merged transcript annotation, as well as the RefSeq annotations of the human and mouse genomes, was used to create the four distinct proteomes described in the main text as follows.

Isoforms were computationally translated into proteins and digested into unique 8-14-mers. Isoforms were translated into proteins “conservatively,” in the sense that the translation was performed assuming that the annotated start codons were used and no stop codon readthrough or internal translation initiation occurred (e.g., generally only the first portion of a retained intron would be translated until an in-frame premature termination codon was encountered, after which translation was assumed to halt). The binding affinity for each resulting peptide to the relevant MHC alleles was then predicted using NetMHCpan v4.0 (Jurtz, V., et al. (2017). NetMHCpan-4.0: Improved Peptide-MHC Class I Interaction Predictions Integrating Eluted Ligand and Peptide Binding Affinity Data. J Immunol 199, 3360-3368, incorporated herein by reference in its entirety). Each peptide was annotated with relevant information about its encoding transcript, including parent gene, parent isoform(s), differential gene and/or isoform expression (if relevant), position within parent transcript, unique assignment to one versus two or more isoforms of the originating splicing event (if relevant), etc.

Four distinct, custom proteomes for subsequent spectra mapping were created (illustrated in FIG. 5B). (1) “full-length proteome”, created using peptides arising from all unique full-length isoforms. (2) “predicted binders”, created by further restricting to unique 8-14-mers that had a NetMHCpan 4.0 percentile rank<2 (the recommended cutoff for binders fromNetMHCpan 4.0). Two versions of this proteome were created, one including only those isoforms derived from differentially retained constitutive introns based on the RNA-seq data, and one including all isoforms derived from constitutive intron retention (constituting an increase in unique 8-14-mers of ˜28%). Analyses used the complete (latter) proteome unless otherwise indicated. (3) “predicted binders+spiked non-binders”, created by augmenting the “predicted binders” proteome with peptides that were predicted to not bind the relevant MHC alleles with high-confidence, defined as having NetMHCpan percentile rank>90, with the number of such non-binders chosen such that they comprised 10% of the final proteome after adding to the “predicted binders” proteome. (4) “filtered predicted binders”, created by further filtering the “predicted binders” proteome by restricting to peptides arising from genes that were significantly differentially expressed or isoforms that were significantly differentially spliced in indisulam-treated versus DMSO-treated samples, defined based on the RNA-seq analysis for the corresponding cell lines.

Peptide Identification from Mass Spectrometry Data

Mass spectra from all MHC immunoprecipitations were analyzed using Proteome Discoverer v2.4.1.15, with the following workflow. Spectra from each replicate were searched against each distinct proteome (described above) as follows. For each proteome, searches were performed with no enzyme specificity, precursor mass tolerance of 10 ppm, and fragment mass tolerance of 0.6 Da. Oxidation (+15.995 Da), phosphorylation (+79.966 Da), and deamidated (+0.984 Da) dynamic modifications were included, in addition to N-terminal glutamate to pyro-glutamate (−17.027 Da). False discovery rate (FDR) estimation was performed computationally using the Percolator software. Peptides reaching the 5% FDR threshold were retained for downstream analyses. For the “full-length” proteome, identified peptides were further restricted to those of length 8-14 amino acids before being used as input for subsequent analyses. For the “predicted binders”, “predicted binders+spiked non-binders”, and “filtered predicted binders” proteomes, peptides corresponding to subsequences of the sequences in the input proteomes were removed before the identified peptides were used for subsequent analyses.

Candidate Neoepitope Identification

As described in the main text, two distinct groups of candidate neoepitopes were selected for subsequent immunization experiments. The first group was based on the intersection between mass spectrometry analyses and RNA-seq analyses. Peptides were first identified using the mass spectrometry analysis described above. These peptides were then restricted to the set of indisulam-specific peptides, where an indisulam-specific peptide was defined as a peptide that was identified in one or more indisulam-treated samples, but not recovered in any DMSO-treated samples. These indisulam-specific peptides were then filtered to retain only those peptides arising from alternative isoforms that were significantly differentially spliced in indisulam-treated versus DMSO-treated cells, and subsequently additionally filtered to require (1) isoform specificity and (2) appropriate direction of differential splicing, with those two criteria defined as follows. (1) An isoform-specific peptide was defined as a peptide which arose exclusively from one isoform associated with a given splicing event (e.g., a peptide from a retained intron event is isoform-specific if it arises from translation of the intronic portion of the unspliced mRNA, or if it arises from translation of the exon-exon junction within the spliced mRNA). This definition means that differential splicing of a given event is predicted to alter levels of the isoform encoding an isoform-specific peptide, and therefore likely similarly alter abundance of the isoform-specific peptide itself. (2) Peptides that exhibit appropriate direction of differential splicing are those isoform-specific peptides which are specifically encoded by differentially spliced isoforms that are promoted by indisulam treatment (e.g., the encoding isoform is present at higher levels in indisulam-treated versus DMSO-treated cells). Isoform-specific peptides were only used for subsequent immunization experiments if their parent isoform was more prevalent in the indisulam treatment, signifying that the peptide is expected to be more abundant in indisulam-treated cells. These criteria yielded 72 peptides, which were subsequently tested in immunization experiments.

The second group of peptides used for immunization experiments was derived by combining evidence from RNA-seq analyses and MHC I binding predictions. This set of peptides was defined using the same criteria described above for the first set (derived by intersecting predictions from mass spectrometry analyses as well as RNA-seq analyses), but without the requirement that peptides be detected as indisulam-specific epitopes via MHC I mass spectrometry. To compensate for the fact that direct protein-level detection was not required, a stringent predicted MHC I binding threshold of rank<0.5 (the NetMHCpan recommended threshold for strong binders) for one or more relevant alleles was applied (versus the more lenient threshold of rank<2 used for other, mass spectrometry-based predictions and analyses). Peptides were additionally restricted to those of lengths between 8 and 11 amino acids, as such lengths are preferred by the studied alleles. The final set of peptides used for subsequent immunization experiments was then derived by additionally requiring that peptides be isoform-specific; arise from genes with expression>5 TPM in corresponding indisulam-treated samples (in order to favor peptides from relatively highly expressed genes); and have a difference in isoform ratio>20% in indisulam-treated versus DMSO-treated samples, and isoform ratio<25% in DMSO-treated samples (in order to restrict to peptides that were associated with more dramatic splicing changes). These criteria yielded 39 peptides, which were subsequently tested in immunization experiments.

Peptide Synthesis

Experimental peptides were individually custom synthesized via the solid-phase method by GenScript (Piscataway, N.J.), with standard removal of trifluoracetic acid and replacement with hydrochloride, purified to >98% by HPLC, and lyophilized for storage. Peptides were reconstituted in DMSO at 10 mg/mL and frozen at −80 C until use.

RMA-S Peptide H-2 Stabilization Assay

RMA-S cells were maintained under standard conditions in RPMI+7.5% FCS for expansion. H-2 stabilization experiments were performed as previously described (Ross, P., et al. (2012). A cell-based MHC stabilization assay for the detection of peptide binding to the canine classical class I molecule, DLA-88. Vet Immunol Immunopathol 150, 206-212, incorporated herein by reference in its entirety). Briefly, RMA-S were exposed to 31° C. and 5% CO₂ conditions overnight, incubated with peptides of interest for 30 minutes at 31° C., and then returned to 37° C. and 5% CO2 for three hours prior to cell surface staining for H-2K^{b} (clone AF6-88.5) and H-2D^{b} molecules (clone KH95) and standard flow cytometry analysis.

TiterMax Immunization

Unless otherwise specified, 10 μg of peptide was emulsified with TiterMax Classic (TiterMax Corp., Norcross, Ga.) and injected into the hocks of anesthetized animals. On day +7 after challenge, draining lymph nodes were collected and CD8⁺ T cells purified by magnetic selection (Miltenyi Biotec, Cat. 130-117-044).

IFNγ ELISpot

CD8⁺ T cells from TiterMax immunized animals were cultured overnight with 20 U/mL mouse IL-2 (PeproTech, Cat. 212-12) and plated at 10⁵ per well in combination with 3×10⁵ T cell depleted syngeneic splenocytes which had been loaded with 100 μg/mL of peptides of interest for 18 hours. PMA 1 μg/mL+ionomycin 500 ng/mL stimulation of T cells served as positive control.

In some experiments, in lieu of peptide-loaded splenocytes, instead ovalbumin-expressing B16-F10 cells or B16-F10 cells treated with DMSO or indisulam 1 μM for 96 hours were stimulated overnight with IFNγ 100 U/mL for the last 24 hours of cell culture. Such cells were then non-enzymatically harvested, washed repeatedly to remove IFNγ, and irradiated to 60 Gy from a ⁶⁰Co source to inhibit growth and further upregulate MHC I. Tumor cells thus generated were counted and incubated with CD8⁺ T cells at identical ratios as for splenocytes (10⁵ CD8⁺ T cells+3×10⁵ melanoma cells). IFNγ ELISpot was performed as per manufacturer's instructions (BD Biosciences, Cat. 551083). Spots were imaged and quantified on an Immunospot® analyzer (Cellular Technology Limited, Cleveland, Ohio).

B16-F10 Co-Culture Cytotoxicity Assay

B16-F10 cells were harvested, counted, and plated at 10⁴ per well in the presence of 100 U/mL IFNγ overnight to upregulate MHC I. After washing, peptides were loaded onto tumor cells at 100 μg/mL, and 10⁶ CD8⁺ T cells from TiterMax immunized animals were added to the tumor cells. 50 U/mL mouse IL-2 was added to this co-culture of tumor cells+CD8⁺ T cells, which was incubated for three days. After washing to remove free (detached) B16-F10 and T cells, viable B16-F10 were harvested, stained (to exclude T and other hematopoietic cells) and absolute cell numbers enumerated via flow cytometry using counting beads according to the manufacturers' instructions (ThermoFisher Cat. C36950).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of enhancing the susceptibility of a cancer cell to an immunotherapeutic agent, comprising contacting the cancer cell with a first agent that modulates RNA splicing.

2. The method of claim 1, wherein the first agent is E7820.

3. The method of claim 2, further comprising contacting the cell with the immunotherapeutic agent or contacting an immune cell with the immunotherapeutic agent and permitting the immune cell to contact the cancer cell, wherein the immunotherapeutic agent is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, Atezolizumab (Tecentriq), Avelumab (Bavencio), Durvalumab (Imfinzi), KN035, CK-301, AUNP12, CA-170, BMS-986189, Ipilimumab (Yervoy), Tremelimumab, and the like.

4. The method of claim 2, further comprising contacting an immune cell with the immunotherapeutic agent and permitting the immune cell to contact the cancer cell, wherein the immunotherapeutic agent is a PD1 inhibitor, optionally an anti-PD1 antibody, optionally is selected from is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, and the like.

5. The method of claim 1, wherein the first agent binds and/or inhibits one of the following RNA splicing factors: SF3B1 (SF3b155), SF3B2 (SF3b145), SF3B3 (SF3b130), SF3B4 (SF3b49), SF3B6 (SF3b14a or p14), PHF5A (SF3b14b), SF3B5 (SF3b10), U2AF1 (U2AF35), and U2AF2 (U2AF65).

6. The method of claim 5, wherein the first agent is selected from E7107, FD-895, FR901464, H3B-8800, herboxidiene (GEX1A), meayamycin, pladienolide B, pladienolide D, spliceostatin A, isoginkgetin, and madrasin.

7. The method of claim 1, wherein the first agent binds, inhibits, and/or degrades via DCAF15 one of the following RNA splicing factors: RBM39 and RBM23.

8. The method of claim 1, wherein the first agent causes degradation of RBM39 and/or RBM23.

9. The method of claim 7 or claim 8, wherein the first agent is selected from indisulam, E7820, tasisulam, or chloroquinoxaline sulfonamide (CQS).

10. The method of claim 1, wherein the first agent directly inhibits post-translational modification of one of the following RNA splicing factors: PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, and SART3.

11. The method of claim 10, wherein the first agent inhibits one of CLK1, CLK2, CLK3, CLK4, SRPK1, DYRK1a, DYRK1b, a Type I PRMT enzyme, and PRMT5, thereby resulting in inhibition of post-translational modification of the RNA splicing factor.

12. The method of claim 11, wherein the Type I PRMT enzyme is selected from PRMT1, PRMT3, PRMT4, PRMT6, and PRMT8.

13. The method of claim 11 or claim 12, wherein the first agent inhibits the Type I PRMT enzyme and is selected from MS-023, TC-E 5003, GSK3368715, and the like.

14. The method of claim 11, wherein the first agent inhibits PRMT5 and is selected from GSK3326595, EPZ015666, LLY-283, JNJ-64619178, PRT543, and the like.

15. The method of any one of claim 1 to claim 14, further comprising contacting the cancer cell with the immunotherapeutic agent or contacting an immune cell with the immunotherapeutic agent and permitting the immune cell to contact the cancer cell.

16. The method of claim 1 or claim 15, wherein the immunotherapeutic agent is a checkpoint inhibitor.

17. The method of claim 16, wherein the checkpoint inhibitor targets PD-1, PD-L1, PD-L2, CTLA-4, CD27, CD28, CD40, CD40L, CD122, CD134 (OX40), CD137 (4-1BB), GITR, ICOS, A2AR, CD276 B7-H3), VTCN1 (B7-H4), TMIGD2, BTLA, IDO, NOX2, CD160, LIGHT, LAG3, DNAM-1, TIGIT, CD96, 2B4, Tim-3, SIRPα, CD200R, DR3, LAG3, VISTA, and the like.

18. The method of claim 17, wherein the checkpoint inhibitor inhibits PD-1 and is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, and the like.

19. The method of claim 17, wherein the checkpoint inhibitor inhibits PD-L1 and is selected from Atezolizumab (Tecentriq), Avelumab (Bavencio), Durvalumab (Imfinzi), KN035, CK-301, AUNP12, CA-170, BMS-986189, and the like.

20. The method of claim 17, wherein the checkpoint inhibitor inhibits CTLA-4 and is selected from Ipilimumab (Yervoy), Tremelimumab, and the like.

21. The method of any one of claim 1 to claim 20, wherein the cancer cell is in vitro.

22. The method of any one of claim 1 to claim 20, wherein the cancer cell is in vivo and contacting the cancer cell comprises administering to the subject a therapeutically effective amount of the agent that modulates RNA splicing.

23. The method of claim 22, further comprising administering to the subject a therapeutically effective amount of a checkpoint inhibitor as recited in one of claim 17 to claim 20.

24. The method of claim 23, wherein the first agent is E7820.

25. The method of claim 1, further comprising contacting the cancer cell with the immunotherapeutic agent or contacting an immune cell with the immunotherapeutic agent and permitting the immune cell to contact the cancer cell.

26. The method of claim 25, wherein the immunotherapeutic agent is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, Atezolizumab (Tecentriq), Avelumab (Bavencio), Durvalumab (Imfinzi), KN035, CK-301, AUNP12, CA-170, BMS-986189, Ipilimumab (Yervoy), Tremelimumab, and the like.

27. The method of claim 26, wherein the first agent is E7820.

28. A method of treating a cancer in a subject in need thereof, comprising administering to the subject a therapeutically effective amount of a first agent that modulates RNA splicing in cancer cells and a therapeutically effective amount of an immunotherapeutic agent.

29. The method of claim 28, wherein the first agent is E7820, and wherein the immunotherapeutic agent is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, Atezolizumab (Tecentriq), Avelumab (Bavencio), Durvalumab (Imfinzi), KN035, CK-301, AUNP12, CA-170, BMS-986189, Ipilimumab (Yervoy), Tremelimumab, and the like.

30. The method of claim 28, wherein the first agent is E7820, and wherein the immunotherapeutic agent is a PD1 inhibitor, optionally an anti-PD1 antibody, optionally is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, and the like.

31. The method of claim 28, wherein the first agent binds and/or inhibits one of the following RNA splicing factors: SF3B1 (SF3b155), SF3B2 (SF3b145), SF3B3 (SF3b130), SF3B4 (SF3b49), SF3B6 (SF3b14a or p14), PHF5A (SF3b14b), SF3B5 (SF3b10), U2AF1 (U2AF35), and U2AF2 (U2AF65).

32. The method of claim 31, wherein the first agent is selected from E7107, FD-895, FR901464, H3B-8800, herboxidiene (GEX1A), meayamycin, pladienolide B, pladienolide D, spliceostatin A, isoginkgetin, and madrasin.

33. The method of claim 28, wherein the first agent binds, inhibits, and/or degrades via DCAF15 one of the following RNA splicing factors: RBM39 and RBM23.

34. The method of claim 28, wherein the first agent causes degradation of RBM39 and/or RBM23.

35. The method of claim 33 or claim 34, wherein the first agent is selected from indisulam, E7820, tasisulam, or chloroquinoxaline sulfonamide (CQS).

36. The method of claim 28, wherein the first agent directly inhibits post-translational modification of one of the following RNA splicing factors: PHF5A, SF3B1, U2AF1, YBX1, RBMX, hnRNPU, hnRNPF, hnRNPH1, ELAVL1, SRRT, hnRNPH2, TRA2B, hnRNPK, PABPN1, DHX9, CWC15, SNRPB, SRSF9, SRRM2, hnRNPA2B1, hnRNPR, LSM4, hnRNPA1, and SART3.

37. The method of claim 36, wherein the first agent inhibits one of CLK1, CLK2, CLK3, CLK4, SRPK1, DYRK1a, DYRK1b, a Type I PRMT enzyme, and PRMT5, thereby resulting in inhibition of post-translational modification of the RNA splicing factor.

38. The method of claim 37, wherein the Type I PRMT enzyme is selected from PRMT1, PRMT3, PRMT4, PRMT6, and PRMT8.

39. The method of claim 37 or claim 38, wherein the first agent inhibits the Type I PRMT enzymes and is selected from MS-023, TC-E 5003, GSK3368715, and the like.

40. The method of claim 37, wherein the first agent inhibits PRMT5 and is selected from GSK3326595, EPZ015666, LLY-283, JNJ-64619178, PRT543, and the like.

41. The method of one of claim 28 and claim 31 to claim 40, wherein the immunotherapeutic agent is a checkpoint inhibitor.

42. The method of claim 41, wherein the checkpoint inhibitor targets PD-1, PD-L1, PD-L2, CTLA-4, CD27, CD28, CD40, CD40L, CD122, CD134 (OX40), CD137 (4-1BB), GITR, ICOS, A2AR, CD276 B7-H3), VTCN1 (B7-H4), TMIGD2, BTLA, IDO, NOX2, CD160, LIGHT, LAG3, DNAM-1, TIGIT, CD96, 2B4, Tim-3, SIRPα, CD200R, DR3, LAG3, VISTA, and the like.

43. The method of claim 42, wherein the checkpoint inhibitor inhibits PD-1 and is selected from Pembrolizumab (Keytruda), Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), AMP-224, AMP-514, and the like.

44. The method of claim 42, wherein the checkpoint inhibitor inhibits PD-L1 and is selected from Atezolizumab (Tecentriq), Avelumab (Bavencio), Durvalumab (Imfinzi) KN035, CK-301, AUNP12, CA-170, BMS-986189, and the like.

45. The method of claim 42, wherein the checkpoint inhibitor inhibits CTLA-4 and is selected from Ipilimumab (Yervoy), Tremelimumab, and the like.

46. The method of one of claim 28 to claim 45, wherein the agent and the immunotherapeutic agent are administered concurrently or within a period of 7 days of each other.
